(19) United States
(12) Patent Application Publication
(10) Pub. No.: US 2006/0148838 A1
(43) Pub. Date: Jul. 6, 2006

(54) Novel pyrrolodihydroisoquinolines useful in the treatment of cancer

(75) Inventors: Matthias Vennemann, Konstanz (DE); Thomas Bar, Reichenau (DE); Jurgen Braunger, Konstanz (DE); Volker Gekeler, Konstanz (DE)

Correspondence Address: NATH & ASSOCIATES PLLC, 112 South West Street, Alexandria, VA, 22314, US

(73) Assignee: Altana Pharma AG, Konstanz (DE)

(21) Appl. No.: 10/562,149

(22) PCT Filed: Jun. 30, 2004
(86) PCT No.: PCT/EP04/51315

§ 371 Date: Dec. 23, 2005

(30) Foreign Application Priority Data
Jun. 30, 2003 (EP) 03014424.0

(51) Int. Cl. A61K 31/4745; C07D 491/02; C07D 451/02

(52) U.S. Cl. 514/284; 514/291; 546/94; 546/62

(57) ABSTRACT

The invention relates to novel pyrrolodihydroisoquinoline derivatives, which are efficacious inhibitors of cellular (hyper)proliferation and/or inducers of apoptosis in cancer cells.

### FIELD OF APPLICATION OF THE INVENTION

The invention relates to novel pyrrolodihydroisoquinoline derivatives, which are used in the pharmaceutical industry for the production of pharmaceutical compositions.

### KNOWN TECHNICAL BACKGROUND

Cancer chemotherapy was established with the alkylating agent Cyclophosphamide (Endoxan®), an oxazaphosphorin pro-drug activated preferentially in the tumor. The target of alkylating agents like Cyclophosphamide is DNA and the concept, that cancer cells with uncontrolled proliferation and a high mitotic index are killed preferentially, proved to be very sucessfull. Standard cancer chemotherapeutic drugs finally kill cancer cells upon induction of programmed cell death (“apoptosis”) by targeting basic cellular processes and molecules. These basic cellular processes and molecules include RNA/DNA (alkylating and carbamylating agents, platin analogs and topoisomerase inhibitors), metabolism (drugs of this class are named anti-metabolites and examples are folic acid, purin and pyrimidine antagonists) as well as the mitotic spindle apparatus with 4-tubulin heterodimers as the essential component (drugs are categorized into stabilizing and destabilizing tubulin inhibitors; examples are Taxol/Paclitaxel®, Docetaxel/Taxotere® and vinca alkaloids).

1. Prior Art

The international applications WO 02/48144, WO 03/014115, WO 03/014116, WO 03/014117 and WO 03/051877 disclose pyrrolodihydroisoquinoline derivatives with PDE10 inhibitory activity. The U.S. Pat. No. 5,965,575 discloses pyrrolodihydroisoquinoline derivatives as 5HT_{1B} antagonists.

2. Description of the invention

It has now been found that the pyrroloisoquinoline derivatives, which are described in greater details below, differ from prior art compounds by unanticipated structural features and have surprising and particularly advantageous properties.

In more detail, thus, for example, it has been unexpectedly and unanticipatedly found that the pyrrolodihydroisoquinoline derivatives, which are described in greater details below, are potent and highly efficacious inhibitors of cellular proliferation and inducers of apoptosis in cancer cells. Therefore, yet unanticipatedly, these pyrrolodihydroisoquinoline derivatives can be useful for treating hyperproliferative diseases and/or disorders responsive to the induction of apoptosis, in particular cancer.

In this context, in further more surprising detail, it has been particularly found that the pyrrolodihydroisoquinoline derivatives, which are described in greater details below, stand out from the general class of the pyrrolodihydroisoquinolines, whose original property is inhibition of PDE10, in interesting and valueable properties, such as e.g. those mentioned afore, i.e. inhibiting cellular (hyper)proliferation and inducing apoptosis in cancer cells, which make them particularly interesting for treating e.g. hyperproliferative diseases and/or disorders responsive to the induction of apoptosis, in particular cancer.

The invention thus relates to compounds of formula I 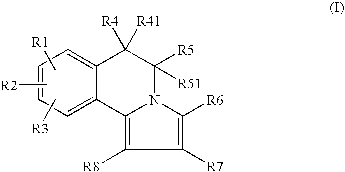
in which
R1 is halogen, nitro, amino, mono- or di-1-4C-alkylamino, 1-4C-alkyl, hydroxyl, 1-4C-alkoxy, 1-4C-alkoxy-2-4C-alkoxy, 3-7C-cycloalkoxy, 3-7C-cycloalkylmethoxy, or completely or predominantly fluorine-substituted 1-4C-alkoxy,
R2 is hydrogen, halogen or 1-4C-alkoxy,
R3 is hydrogen or 1-4C-alkoxy, or
R2 and R3 bound to the benzo ring moiety in ortho-position to each other together form a 1-2C-alkylenedioxy bridge, or
R2 and R3 bound to the benzo ring moiety in ortho-position to each other together form a completely or predominantly fluorine-substituted 1-2C-alkylenedioxy bridge, or
R1 and R2 bound to the benzo ring moiety in ortho-position to each other together form a 1-2C-alkylenedioxy bridge and R3 is hydrogen, or
R1 and R2 bound to the benzo ring moiety in ortho-position to each other together form a completely or predominantly fluorine-substituted 1-2C-alkylenedioxy bridge and R3 is hydrogen,
R4 is hydrogen, fluorine, chlorine, 1-4C-alkyl, trifluoromethyl, cyclopropyl, cyano, 1-4C-alkoxycarbonyl or —CH₂O—R411, in which
R411 is hydrogen, 1-4C-alkyl, 1-4C-alkoxy-2-4-alkyl or 1-4C-alkylcarbonyl,
R41 is hydrogen or 1-4C-alkyl,
R5 is hydrogen,
R51 is hydrogen, or
R4 is hydrogen, fluorine, chlorine or 1-4C-alkyl,
R41 is hydrogen or 1-4C-alkyl,
R5 is hydrogen,
R51 is hydrogen,
R6 is 1-6C-alkyl, amino, formyl, or 1-4C-alkyl substituted by R61, in which
R61 is 1-4C-alkoxycarbonyl, carboxyl, 1-4C-alkoxy, hydroxyl, halogen or —N(R611)R612, in which
R611 is hydrogen, 1-4C-alkyl, 3-7C-cycloalkyl or 3-7C-cycloalkyl-1-4C-alkyl,
R612 is hydrogen or 1-4C-alkyl, or
R611 and R612 together and with inclusion of the nitrogen atom to which they are bound form a radical Het1, in which
Het1 is a 5- to 7-membered saturated heterocyclic ring radical comprising one nitrogen atom, to which R611 and R612 are bound, and, optionally, one further heteroatom selected from a group consisting of nitrogen, oxygen and sulfur, and optionally substituted by R613 on a ring nitrogen atom, in which
R613 is 1-4C-alkyl, 3-7C-cycloalkyl, 3-7C-cycloalkyl-1-4C-alkyl, hydroxy-2-4C-alkyl, 1-4C-alkoxy-2-4C-alkyl, amino-2-4C-alkyl, mono- or di-1-4C-alkylamino-2-4C-alkyl, formyl, pyridyl or pyrimidinyl,
R7 is phenyl, Het2, R71- and/or R72- and/or R73-substituted phenyl, R74- and/or R75-substituted Het2, naphthyl, or R76- and/or R77-substituted naphthyl, in which
Het2 is a monocyclic or fused bicyclic 5 to 10-membered heteroaryl radical comprising one to three heteroatoms, each of which is selected from a group consisting of nitrogen, oxygen and sulfur,
R71 is hydroxyl, halogen, nitro, cyano, trifluoromethyl, 1-4C-alkyl, 1-4C-alkoxy, amino, mono- or di-1-4C-alkylamino, 1-4C-alkylsulphonylamino, arylsulphonylamino, 1-4C-alkoxycarbonyl, carboxyl, 1-4C-alkylthio, aryloxy-2-4C-alkoxy, aryloxy-1-4C-alkyl, aryloxy, aryl-1-4C-alkoxy, aryl, 1-4C-alkoxy-2-4C-alkoxy, 1-4C-alkoxy-1-4C-alkyl, hydroxy-2-4C-alkoxy, amino-2-4C-alkoxy, mono- or di-1-4C-alkylamino-2-alkoxy, or completely or predominantly fluorine substituted 1-4C-alkoxy, in which
aryl is phenyl or R711-substituted phenyl, in which
R711 is halogen, 1-4C-alkyl, 1-4C-alkoxy, nitro or cyano,
R72 is halogen, 1-4C-alkyl, 1-4C-alkoxy or 1-4C-alkoxycarbonyl,
R73 is 1-4C-alkyl or 1-4C-alkoxy,
R74 is halogen, 1-4C-alkyl, trifluoromethyl, 1-4C-alkoxy, cyano, amino, mono- or di-1-4C-alkylamino, 1-4C-alkoxycarbonyl, morpholino, carboxyl, nitro, phenyl or phenyloxy,
R75 is 1-4C-alkyl or halogen,
R76 is halogen, hydroxyl, 1-4C-alkyl, 1-4C-alkoxy, carboxyl or 1-4C-alkoxycarbonyl,
R77 is 1-4C-alkyl or 1-4C-alkoxy,
R8 is phenyl, phenylcarbonyl, —C(O)—N(R82)R83 or —C(O)—OR9, in which
R82 is hydrogen, 1-4C-alkyl, 3-7C-cycloalkyl, 3-7C-cycloalkyl-1-4C-alkyl, phenyl or phenyl-1-4C-alkyl,
R83 is hydrogen or 1-4C-alkyl, or
R82 and R83 together and with inclusion of the nitrogen atom, to which they are bound, form a heterocyclic ring radical selected from the group consisting of pyrrolidinyl, piperidinyl, morpholinyl or N-(1-4C-alkyl)-piperazinyl,
R9 is 1-4C-alkyl;
   under the provisio, that this subgroup of compounds of formula I,
   wherein the combination of all of the following restrictions a.) to c.) apply, is thereof disclaimed:
a.) the substitution pattern of the left R1- and/or R2- and/or R3-substituted benzo ring of the dihydroisoquinoline moiety of the pyrrolodihydroisoquinoline scaffold shown in formula I is as follows: 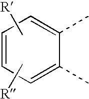
   in which
   R′ and R″ can be bonded at any possible position of the benzo ring, and
   R′ is hydroxyl, 1-4C-alkoxy or trifluoromethoxy,
   R″ is hydrogen or 1-4C-alkoxy,
   or R′ and R″ bound to the benzo ring moiety in ortho-position to each other together form a 1-2C-alkylenedioxy bridge,
      and
b.) R4 is hydrogen, and
   R41 is hydrogen, and
   R5 is hydrogen, and
   R51 is hydrogen,
      and
c.) R8 is —C(O)—OR9, in which
   R9 is 1-4C-alkyl;
      and to the salts, stereoisomers, hydrates and hydrates of the salts of these compounds.

The invention further relates in a first aspect (aspect a) to compounds of formula I, in which
R1 is halogen, nitro, amino, mono- or di-1-4C-alkylamino, 1-4C-alkyl, hydroxyl, 1-4C-alkoxy, 1-4C-alkoxy-2-4C-alkoxy, 3-7C-cycloalkoxy, 3-7C-cycloalkylmethoxy, or completely or predominantly fluorine-substituted 1-4C-alkoxy,
R2 is hydrogen, halogen or 1-4C-alkoxy,
R3 is hydrogen or 1-4C-alkoxy, or
R2 and R3 bound to the benzo ring moiety in ortho-position to each other together form a 1-2C-alkylenedioxy bridge, or
R2 and R3 bound to the benzo ring moiety in ortho-position to each other together form a completely or predominantly fluorine-substituted 1-2C-alkylenedioxy bridge, or
R1 and R2 bound to the benzo ring moiety in ortho-position to each other together form a 1-2C-alkylenedioxy bridge and R3 is hydrogen, or
R1 and R2 bound to the benzo ring moiety in ortho-position to each other together form a completely or predominantly fluorine-substituted 1-2C-alkylenedioxy bridge and R3 is hydrogen,
R4 is hydrogen, fluorine, chlorine, 1-4C-alkyl, trifluoromethyl, cyclopropyl, cyano, 1-4C-alkoxycarbonyl or —CH₂O—R411, in which
R411 is hydrogen, 1-4C-alkyl, 1-4C-alkoxy-2-4-alkyl or 1-4C-alkylcarbonyl,
R41 is hydrogen or 1-4C-alkyl,
R5 is hydrogen,
R51 is hydrogen,
   or
R4 is hydrogen, fluorine, chlorine or 1-4C-alkyl,
R41 is hydrogen or 1-4C-alkyl,
R5 is hydrogen,
R51 is hydrogen,
R6 is 1-6C-alkyl, amino, formyl, or 1-4C-alkyl substituted by R61, in which
R61 is 1-4C-alkoxycarbonyl, carboxyl, 1-4C-alkoxy, hydroxyl, halogen or —N(R611)R612, in which
R611 is hydrogen, 1-4C-alkyl, 3-7C-cycloalkyl or 3-7C-cycloalkyl-1-4C-alkyl,
R612 is hydrogen or 1-4C-alkyl, or
R611 and R612 together and with inclusion of the nitrogen atom to which they are bound form a radical Het1, in which
Het1 is a 5- to 7-membered saturated heterocyclic ring radical comprising one nitrogen atom, to which R611 and R612 are bound, and, optionally, one further heteroatom selected from a group consisting of nitrogen, oxygen and sulfur, and optionally substituted by R613 on a ring nitrogen atom, in which
R613 is 1-4C-alkyl, 3-7C-cycloalkyl, 3-7C-cycloalkyl-1-4C-alkyl, hydroxy-2-4C-alkyl, 1-4C-alkoxy-2-4C-alkyl, amino-2-4C-alkyl, mono- or di-1-4C-alkylamino-2-4C-alkyl, formyl, pyridyl or pyrimidinyl,
R7 is phenyl, Het2, R71- and/or R72- and/or R73-substituted phenyl, R74- and/or R75-substituted Het2, naphthyl, or R76- and/or R77-substituted naphthyl, in which
Het2 is a monocyclic or fused bicyclic 5 to 10-membered heteroaryl radical comprising one to three heteroatoms, each of which is selected from a group consisting of nitrogen, oxygen and sulfur,
R71 is hydroxyl, halogen, nitro, cyano, trifluoromethyl, 1-4C-alkyl, 14-alkoxy, amino, mono- or di-1-4C-alkylamino, 1-4C-alkylsulphonylamino, arylsulphonylamino, 1-4C-alkoxycarbonyl, carboxyl, 1-4C-alkylthio, aryloxy-2-4C-alkoxy, aryloxy-1-4C-alkyl, aryloxy, aryl-1-4C-alkoxy, aryl, 1-4C-alkoxy-2-4C-alkoxy, 1-4C-alkoxy-1-4C-alkyl, hydroxy-2-4C-alkoxy, amino-2-4C alkoxy, mono- or di-1-4C-alkylamino-2-4C-alkoxy, or completely or predominantly fluorine-substituted 1-4C-alkoxy, in which
aryl is phenyl or R711-substituted phenyl, in which
R711 is halogen, 1-4C-alkyl, 1-4C-alkoxy, nitro or cyano,
R72 is halogen, 1-4C-alkyl, 1-4C-alkoxy or 1-4C-alkoxycarbonyl,
R73 is 1-4C-alkyl or 1-4C-alkoxy,
R74 is halogen, 1-4C-alkyl, trifluoromethyl, 1-4C-alkoxy, cyano, amino, mono- or di-1-4C-alkylamino, 1-4C-alkoxycarbonyl, morpholino, carboxyl, nitro, phenyl or phenyloxy,
R75 is 1-4C-alkyl or halogen,
R76 is halogen, hydroxyl, 1-4C-alkyl, 1-4C-alkoxy, carboxyl or 1-4C-alkoxycarbonyl,
R77 is 1-4C-alkyl or 1-4C-alkoxy,
R8 is phenyl, phenylcarbonyl or —C(O)—N(R82)R83, in which
R82 is hydrogen, 1-4C-alkyl, 3-7C-cycloalkyl, 3-7C-cycloalkyl-1-4C-alkyl, phenyl or phenyl-1-4C-alkyl,
R83 is hydrogen or 1-4C-alkyl, or
R82 and R83 together and with inclusion of the nitrogen atom, to which they are bound, form a heterocyclic ring radical selected from the group consisting of pyrrolidinyl, piperidinyl, morpholinyl or N-(1-4C-alkyl)-piperazinyl,
   and to the salts, stereoisomers, hydrates and hydrates of the salts of these compounds.

The invention further relates in a second aspect (aspect b) to compounds of formula I, in which
R1 is halogen, nitro, amino, mono- or di-1-4C-alkylamino, 1-4C-alkyl, 1-4C-alkoxy-2-4C-alkoxy, 3-7C-cycloalkoxy, 3-7C-cycloalkylmethoxy, or completely or predominantly fluorine-substituted 1-4C-alkoxy,
   with the provisio that R1 is not trifluoromethoxy,
R2 is hydrogen, halogen or 1-4C-alkoxy,
R3 is hydrogen or 1-4C-alkoxy, or
R2 and R3 bound to the benzo ring moiety in ortho-position to each other together form a 1-2C-alkylenedioxy bridge, or
R2 and R3 bound to the benzo ring moiety in ortho-position to each other together form a completely or predominantly fluorine-substituted 1-2C-alkylenedioxy bridge, or
R1 and R2 bound to the benzo ring moiety in ortho-position to each other together form a 1-2C-alkylenedioxy bridge and R3 is hydrogen, or
R1 and R2 bound to the benzo ring moiety in ortho-position to each other together form a completely or predominantly fluorine-substituted 1-2C-alkylenedioxy bridge and R3 is hydrogen,
R4 is hydrogen, fluorine, chlorine, 1-4C-alkyl, trifluoromethyl, cyclopropyl, cyano, 1-4C-alkoxycarbonyl or CH₂O—R411, in which
R411 is hydrogen, 1-4C-alkyl, 1-4C-alkoxy-2-4C-alkyl or 1-4C-alkylcarbonyl,
R41 is hydrogen or 1-4C-alkyl,
R5 is hydrogen,
R51 is hydrogen,
   or
R4 is hydrogen, fluorine, chlorine or 1-4C-alkyl,
R41 is hydrogen or 1-4C-alkyl,
R5 is hydrogen,
R51 is hydrogen,
R6 is 1-6C-alkyl, amino, formyl, or 1-4C-alkyl substituted by R61, in which
R61 is 1-4C-alkoxycarbonyl, carboxyl, 1-4C-alkoxy, hydroxyl, halogen or —N(R611)R612, in which
R611 is hydrogen, 1-4C-alkyl, 3-7C-cycloalkyl or 3-7C-cycloalkyl-1-4C-alkyl,
R612 is hydrogen or 1-4C-alkyl, or
R611 and R612 together and with inclusion of the nitrogen atom to which they are bound form a radical Het1, in which
Het1 is a 5 to 7-membered saturated heterocyclic ring radical comprising one nitrogen atom, to which R611 and R612 are bound, and, optionally, one further heteroatom selected from a group consisting of nitrogen, oxygen and sulfur, and optionally substituted by R613 on a ring nitrogen atom, in which
R613 is 1-4C-alkyl, 3-7C-cycloalkyl, 3-7C-cycloalkyl-1-4C-alkyl, hydroxy-2-4C-alkyl, 1-4C-alkoxy-2-4C-alkyl, amino-2-4C-alkyl, mono- or di-1-4C-alkylamino-2-4C-alkyl, formyl, pyridyl or pyrimidinyl,
R7 is phenyl, Het2, R71- and/or R72- and/or R73-substituted phenyl, R74- and/or R75-substituted Het2, naphthyl, or R76- and/or R77-substituted naphthyl, in which
Het2 is a monocyclic or fused bicyclic 5 to 10-membered heteroaryl radical comprising one to three heteroatoms, each of which is selected from a group consisting of nitrogen, oxygen and sulfur,
R71 is hydroxyl, halogen, nitro, cyano, trifluoromethyl, 1-4C-alkyl, 1-4C-alkoxy, amino, mono- or di-1-4C-alkylamino, 1-4C-alkylsulphonylamino, arylsulphonylamino, 1-4C-alkoxycarbonyl, carboxyl, 1-4C-alkylthio, aryloxy-2-4C-alkoxy, aryloxy-1-4C-alkyl, aryloxy, aryl-1-4C-alkoxy, aryl, 1-4C-alkoxy-2-4C-alkoxy, 1-4C-alkoxy-1 alkyl, hydroxy-2-4C-alkoxy, amino-2-4C-alkoxy, mono- or di-1-4C-alkylamino-2-4C-alkoxy, or completely or predominantly fluorine-substituted 1-4C-alkoxy, in which aryl is phenyl or R711-substituted phenyl, in which
R711 is halogen, 1-4C-alkyl, 1-4C-alkoxy, nitro or cyano,
R72 is halogen, 1-4C-alkyl, 1-4C-alkoxy or 1-4C-alkoxycarbonyl,
R73 is 1-4C-alkyl or 1-4C-alkoxy,
R74 is halogen, 1-4C-alkyl, trifluoromethyl, 1-4C-alkoxy, cyano, amino, mono- or di-1-4C-alkylamino, 1-4C-alkoxycarbonyl, morpholino, carboxyl, nitro, phenyl or phenyloxy,
R75 is 1-4C-alkyl or halogen,
R76 is halogen, hydroxyl, 1-4C-alkyl, 1-4C-alkoxy, carboxyl or 1-4C-alkoxycarbonyl,
R77 is 1-4C-alkyl or 1-4C-alkoxy,
R8 is phenyl, phenylcarbonyl, —C(O)—N(R82)R83 or C(O)—OR9, in which
R82 is hydrogen, 1-4C-alkyl, 3-7C-cycloalkyl, 3-7C-cycloalkyl-1-4C-alkyl, phenyl or phenyl-1-4C-alkyl,
R83 is hydrogen or 1-4C-alkyl, or
R82 and R83 together and with inclusion of the nitrogen atom, to which they are bound, form a heterocyclic ring radical selected from the group consisting of pyrrolidinyl, piperidinyl, morpholinyl or N-(1-4C-alkyl)-piperazinyl,
R9 is 1-4C-alkyl,
   and to the salts, stereoisomers, hydrates and hydrates of the salts of these compounds.

The invention further relates in a third aspect (aspect c) to compounds of formula I, in which
R1 is halogen, nitro, amino, mono- or di-1-4C-alkylamino, 1-4C-alkyl, hydroxyl, 1-4C-alkoxy, 1-4C-alkoxy-2-4C-alkoxy, 3-7C-cycloalkoxy, 3-7C-cycloalkylmethoxy, or completely or predominantly fluorine-substituted 1-4C-alkoxy,
R2 is halogen or 1-4C-alkoxy,
R3 is 1-4C-alkoxy, or
R2 and R3 bound to the benzo ring moiety in ortho-position to each other together form a 1-2C-alkylenedioxy bridge, or
R2 and R3 bound to the benzo ring moiety in ortho-position to each other together form a completely or predominantly fluorine-substituted 1-2C-alkylenedioxy bridge,
R4 is hydrogen, fluorine, chlorine, 1-4C-alkyl, trifluoromethyl, cyclopropyl, cyano, 1-4C-alkoxycarbonyl or —CH₂—O—R411, in which
R411 is hydrogen, 1-4C-alkyl, 1-4C-alkoxy-2-4-alkyl or 1-4C-alkylcarbonyl,
R41 is hydrogen or 1 alkyl,
R5 is hydrogen,
R51 is hydrogen,
   or
R4 is hydrogen, fluorine, chlorine or 1-4C-alkyl,
R41 is hydrogen or 1-4C-alkyl,
R5 is hydrogen,
R51 is hydrogen,
R6 is 1-4C-alkyl, amino, formyl, or 1-4C-alkyl substituted by R61, in which
R61 is 1-4C-alkoxycarbonyl, carboxyl, 1-4C-alkoxy, hydroxyl, halogen or —N(R611)R612, in which
R611 is hydrogen, 1-4C-alkyl, 3-7C-cycloalkyl or 3-7C-cycloalkyl-1-4C-alkyl,
R612 is hydrogen or 1-4C-alkyl, or
R611 and R612 together and with inclusion of the nitrogen atom to which they are bound form a radical Het1, in which
Het1 is a 5- to 7-membered saturated heterocyclic ring radical comprising one nitrogen atom, to which R611 and R612 are bound, and, optionally, one further heteroatom selected from a group consisting of nitrogen, oxygen and sulfur, and optionally substituted by R613 on a ring nitrogen atom, in which
R613 is 1-4C-alkyl, 3-7C-cycloalkyl, 3-7C-cycloalkyl-1-4C-alkyl, hydroxy-2-4C-alkyl, 1C-alkoxy-2-4C-alkyl, amino-2-4C-alkyl, mono- or di-1-4C-alkylamino-2-4C-alkyl, formyl, pyridyl or pyrimidinyl,
R7 is phenyl, Het2, R71- and/or R72- and/or R73-substituted phenyl, R74 and/or R75-substituted Het2, naphthyl, or R76- and/or R77-substituted naphthyl, in which
Het2 is a monocyclic or fused bicyclic 5 to 10-membered heteroaryl radical comprising one to three heteroatoms, each of which is selected from a group consisting of nitrogen, oxygen and sulfur,
R71 is hydroxyl, halogen, nitro, cyano, trifluoromethyl, 1-4C-alkyl, 1-4C-alkoxy, amino, mono- or di-1-4C-alkylamino, 1-4C-alkylsulphonylamino, arylsulphonylamino, 1-4C-alkoxycarbonyl, carboxyl, 1-4C-alkylthio, aryloxy-2-4C-alkoxy, aryloxy-1-4C-alkyl, aryloxy, aryl-1-4C-alkoxy, aryl, 1-4C-alkoxy-2-4C-alkoxy, 1-4C-alkoxy-1-4C-alkyl, hydroxy-2-4C-alkoxy, amino-2-4C-alkoxy, mono- or di-1-4C-alkylamino-2-4C-alkoxy, or completely or predominantly fluorine-substituted 1-4C-alkoxy, in which
aryl is phenyl or R711-substituted phenyl, in which
R711 is halogen, 1-4C-alkyl, 1-4C-alkoxy, nitro or cyano,
R72 is halogen, 1-4C-alkyl, 1-4C-alkoxy or 1-4C-alkoxycarbonyl,
R73 is 1-4C-alkyl or 1-4C-alkoxy,
R74 is halogen, 1-4C-alkyl, trifluoromethyl, 1-4C-alkoxy, cyano, amino, mono- or di-1-4C-alkylamino, 1-4C-alkoxycarbonyl, morpholino, carboxyl, nitro, phenyl or phenyloxy,
R75 is 1-4C-alkyl or halogen,
R76 is halogen, hydroxyl, 1-4C-alkyl, 1-4C-alkoxy, carboxyl or 1-4C-alkoxycarbonyl,
R77 is 1-4C-alkyl or 1-4C-alkoxy,
R8 is phenyl, phenylcarbonyl, —C(O)—N(R82)R83 or —C(O)—OR9, in which
R82 is hydrogen, 1-4C-alkyl, 3-7C-cycloalkyl, 3-7C-cycloalkyl-1-4C-alkyl, phenyl or phenyl-1-4C-alkyl,
R83 is hydrogen or 1-4C-alkyl, or
R82 and R83 together and with inclusion of the nitrogen atom, to which they are bound, form a heterocyclic ring radical selected from the group consisting of pyrrolidinyl, piperidinyl, morpholinyl or N-1-4C-alkyl)-piperazinyl,
R9 is 1-4C-alkyl,
   and to the salts, stereoisomers, hydrates and hydrates of the salts of these compounds.

The invention further relates in a fourth aspect (aspect d) to compounds of formula I, in which
R1 is halogen, nitro, amino, mono- or di-1-4C-alkylamino, 1-4C-alkyl, hydroxyl, 1-4C-alkoxy, 1-4C-alkoxy-2-4C-alkoxy, 3-7C-cycloalkoxy, 3-7C-cycloalkylmethoxy, or completely or predominantly fluorine-substituted 1-4C-alkoxy,
R2 is hydrogen, halogen or 1-4C-alkoxy,
R3 is hydrogen or 1-4C-alkoxy, or
R2 and R3 bound to the benzo ring moiety in ortho-position to each other together form a 1-2C-alkylenedioxy bridge, or
R2 and R3 bound to the benzo ring moiety in ortho-position to each other together form a completely or predominantly fluorine-substituted 1-2C-alkylenedioxy bridge, or
R1 and R2 bound to the benzo ring moiety in ortho-position to each other together form a 1-2C-alkylenedioxy bridge and R3 is hydrogen, or
R1 and R2 bound to the benzo ring moiety in ortho-position to each other together form a completely or predominantly fluorine-substituted 1-2C-alkylenedioxy bridge and R3 is hydrogen,
R4 is fluorine, chlorine, 1-4C-alkyl, trifluoromethyl, cyclopropyl, cyano, 1-4C-alkoxycarbonyl or —CH₂—O—R411, in which
R411 is hydrogen, 1-4C-alkyl, 1-4C-alkoxy-2-4C-alkyl or 1-4C-alkylcarbonyl,
R41 is hydrogen or 1-4C-alkyl,
R5 is hydrogen,
R51 is hydrogen,
R6 is 1-6C-alkyl, amino, formyl, or 1-4C-alkyl substituted by R61, in which
R61 is 1-4C-alkoxycarbonyl, carboxyl, 1-4C-alkoxy, hydroxyl, halogen or —N(R611)R612, in which
R611 is hydrogen, 1-4C-alkyl, 3-7C-cycloalkyl or 3-7C-cycloalkyl-1-4C-alkyl,
R612 is hydrogen or 1-4C-alkyl, or
R611 and R612 together and with inclusion of the nitrogen atom to which they are bound form a radical Het1, in which
Het1 is a 5- to 7-membered saturated heterocyclic ring radical comprising one nitrogen atom, to which R611 and R612 are bound, and, optionally, one further heteroatom selected from a group consisting of nitrogen, oxygen and sulfur, and optionally substituted by R613 on a ring nitrogen atom, in which
R613 is 1-4C-alkyl, 3-7C-cycloalkyl, 3-7C-cycloalkyl-1-4C-alkyl, hydroxy-2-4C-alkyl, 1-4C-alkoxy-2-4C-alkyl, amino-2-4C-alkyl, mono- or di-1-4C-alkylamino-2-4C-alkyl, formyl, pyridyl or pyrimidinyl,
R7 is phenyl, Het2, R71- and/or R72- and/or R73-substituted phenyl, R74 and/or R75-substituted Het2, or naphthyl, or R76- and/or R77-substituted naphthyl, in which
Het2 is a monocyclic or fused bicyclic 5 to 10-membered heteroaryl radical composing one to three heteroatoms, each of which is selected from a group consisting of nitrogen, oxygen and sulfur,
R71 is hydroxyl, halogen, nitro, cyano, trifluoromethyl, 1-4C-alkyl, 1-4C-alkoxy, amino, mono- or di-1-4C-alkylamino, 1-4C-alkylsulphonylamino, arylsulphonylamino, 1-4C-alkoxycarbonyl, carboxyl, 1-4C-alkylthio, aryloxy-2-4C-alkoxy, aryloxy-1-4C-alkyl, aryloxy, aryl-1-4C-alkoxy, aryl, 1-4C-alkoxy-2-4C-alkoxy, 1-4C-alkoxy-1-4C-alkyl, hydroxy-2-4C-alkoxy, amino-2-4C-alkoxy, mono- or di-1-4C-alkylamino-2-4C-alkoxy, or completely or predominantly fluorine-substituted 1-4C-alkoxy, in which
aryl is phenyl or R711-substituted phenyl, in which
R711 is halogen, 1-4C-alkyl, 1-4C-alkoxy, nitro or cyano,
R72 is halogen, 1-4C-alkyl, 1-4C-alkoxy or 1-4C-alkoxycarbonyl,
R73 is 1-4C-alkyl or 1-4C-alkoxy,
R74 is halogen, 1-4C-alkyl, trifluoromethyl, 1-4C-alkoxy, cyano, amino, mono- or di-1-4C-alkylamino, 1-4C-alkoxycarbonyl, morpholino, carboxyl, nitro, phenyl or phenyloxy,
R75 is 1-4C-alkyl or halogen,
R76 is halogen, hydroxyl, 1-4C-alkyl, 1-4C-alkoxy, carboxyl or 1-4C-alkoxycarbonyl,
R77 is 1-4C-alkyl or 1-4C-alkoxy,
R8 is phenyl, phenylcarbonyl, —C(O)—N(R82)R83 or —C(O)—OR9, in which
R82 is hydrogen, 1-4C-alkyl, 3-7C-cycloalkyl, 3-7C-cycloalkyl-1-4C-alkyl, phenyl or phenyl-1-4C-alkyl,
R83 is hydrogen or 1-4C-alkyl, or
R82 and R83 together and with inclusion of the nitrogen atom, to which they are bound, form a heterocyclic ring radical selected from the group consisting of pyrrolidinyl, piperidinyl, morpholinyl or N-(1-4C-alkyl)-piperazinyl,
R9 is 1-4C-alkyl,
   and to the salts, stereoisomers, hydrates and hydrates of the salts of these compounds.

1-4C-Alkyl represents a straight-chain or branched alkyl radical having 1 to 4 carbon atoms. Examples which may be mentioned are the butyl, isobutyl, sec-butyl, tert-butyl, propyl, isopropyl and preferably the ethyl and methyl radicals.

2-4C-Alkyl represents a straight-chain or branched alkyl radical having 2 to 4 carbon atoms. Examples which may be mentioned are the butyl, isobutyl, sec-butyl, tert-butyl, propyl, isopropyl and preferably the ethyl radical.

1-6C-Alkyl represents a straight-chain or branched alkyl radical having 1 to 6 carbon atoms. Examples which may be mentioned are the hexyl, isohexyl (4-methylpentyl), neohexyl (3,3-dimethylbutyl), pentyl, isopentyl (3-methylbutyl), neopentyl (2,2-dimethylpropyl), butyl, isobutyl, sec-butyl, tert-butyl, propyl, isopropyl, ethyl or methyl radicals.

1-4C-Alkoxy represents radicals which, in addition to the oxygen atom, contain a straight-chain or branched alkyl radical having 1 to 4 carbon atoms. Examples which may be mentioned are the butoxy, isobutoxy, sec-butoxy, tert-butoxy, propoxy, isopropoxy and preferably the ethoxy and methoxy radicals.

1-4C-Alkylthio represents radicals which, in addition to the sulfur atom, contain a straight-chain or branched alkyl radical having 1 to 4 carbon atoms. Examples which may be mentioned are the ethylthio and the methylthio radicals.

2-4C-Alkoxy represents radicals which, in addition to the oxygen atom, contain a straight-chain or branched alkyl radical having 2 to 4 carbon atoms. Examples which may be mentioned are the butoxy, isobutoxy, sec-butoxy, tert-butoxy, propoxy, isopropoxy and preferably the ethoxy radical.

3-7C-Cycloalkoxy represents cyclopropyloxy, cyclobutyloxy, cyclopentyloxy, cyclohexyloxy and cycloheptyloxy, of which cyclopropyloxy, cyclobutyloxy and cyclopentyloxy are preferred.

3-7C-Cycloalkyl represents cyclopropyl, cyclobutyl, cyclopentyl, cyclohexyl and cycloheptyl, of which cyclopropyl, cyclobutyl and cyclopentyl are preferred.

3-7C-cycloalkylmethoxy represents cyclopropylmethoxy, cyclobutylmethoxy, cyclopentylmethoxy, cyclohexylmethoxy and cycloheptylmethoxy, of which cyclopropylmethoxy, cyclobutylmethoxy and cyclopentylmethoxy are preferred.

3-7C-Cycloalkyl-1-4C-alkyl represents one of the abovementioned 1-4C-alkyl radicals, which is substituted by one of the abovementioned 3-7C-cycloalkyl radicals. Examples which may be mentioned are the cyclopropylmethyl, the cyclohexylethyl and the cyclohexylmethyl radicals.

As completely or predominantly fluorine-substituted 1-4C-alkoxy, for example, the 2,2,3,3,3-penta-fluoropropoxy, the perfluoroethoxy, the 1,2,2-trifluoroethoxy, in particular the 1,1,2,2-tetrafluoroethoxy, the 2,2,2-trifluoroethoxy, the trifluoromethoxy and preferably the difluoromethoxy radicals may be mentioned. “Predominantly” in this connection means that more than half of the hydrogen atoms of the 1-4C-alkoxy radicals are replaced by fluorine atoms.

1-4C-Alkoxy-2-4C-alkoxy represents one of the abovementioned 2-4C-alkoxy radicals, which is substituted by one of the abovementioned 1-4C-alkoxy radicals. Examples which may be mentioned are the 2-methoxyethoxy, 2-ethoxyethoxy and the 2-isopropoxyethoxy radicals.

1-4C-Alkoxy-2-4C-alkyl represents one of the abovementioned 2-4C-alkyl radicals, which is substituted by one of the abovementioned 1-4C-alkoxy radicals. Examples which may be mentioned are the 2-methoxyethyl and the 2-isopropoxyethyl radicals.

1-4C-Alkoxy-1-4C-alkyl stands for one of the abovementioned 1-4C-alkyl radicals, which is substituted by one of the abovementioned 1-4C-alkoxy radicals. Examples which may be mentioned are the 2-methoxyethyl and 2-isopropoxyethyl radicals.

1-2C-Alkylenedioxy represents, for example, the methylenedioxy [—O—CH₂—O—] and the ethylenedioxy [—O—CH₂—CH₂—O—] radicals.

As completely or predominantly fluorine-substituted 1-2C-alkylenedioxy bridge, for example, the difluoromethylenedioxy [—O—CF₂—O—] radical may be mentioned. “Predominantly” in this connection means that more than half of the hydrogen atoms of the 1-4C-alkylenedioxy radical are replaced by fluorine atoms.

Phenyl-1-4C-alkyl stands for one of the abovementioned 1-4C-alkyl radicals, which is substituted by a phenyl radical. Examples which may be mentioned are the phenethyl and the benzyl radicals.

1-4C-alkoxycarbonyl represents a radical which, in addition to the carbonyl group, contains one of the abovementioned 1-4C-alkoxy radicals. Examples which may be mentioned are the methoxycarbonyl and ethoxycarbonyl radicals.

1-4C-Alkylcarbonyl represents a radical which, in addition to the carbonyl group, contains one of the abovementioned 1-4C-alkyl radicals. An example which may be mentioned is the acetyl radical.

1-4C-Alkylene is a straight-chain alkylene radical such as, for example, the methylene (—CH₂—) or, particularly, the trimethylene (—CH₂—CH₂—CH₂—) or the tetramethylene (—CH₂CH₂—CH₂—CH₂—) radical.

Halogen within the meaning of the invention is bromine and, preferably, chlorine and fluorine.

Hydroxy-2-4C-alkyl stands for one of the abovementioned 2-4C-alkyl radicals which is substituted by a hydroxyl group. Examples which may be mentioned are the 2-hydroxyethyl and 3-hydroxypropyl radicals.

Hydroxy-2-4C-alkoxy stands for one of the abovementioned 2-4C-alkoxy radicals which is substituted by a hydroxyl group. Examples which may be mentioned are the 2-hydroxyethoxy and 3-hydroxypropoxy radicals.

Amino-2-4C-alkyl stands for one of the abovementioned 2-4C-alkyl radicals which is substituted by an amino group. Examples which may be mentioned are the 2-aminoethyl and 3-aminopropyl radicals.

Amino-2-4C-alkoxy stands for one of the abovementioned 2-4C-alkoxy radicals which is substituted by an amino group. Examples which may be mentioned are the 2-aminoethoxy and 3-aminopropoxy radicals.

In addition to the nitrogen atom, mono- or di-1-4C-alkylamino radicals contain one or two of the abovementioned 1-4C-alkyl radicals. Di-1-4C-alkylamino is to be emphasized and here, in particular, dimethyl-, diethyl- and diisopropylamino.

Mono- or Di-1-4C-alkylamino-2-4C-alkyl stands for one of the abovementioned 2-4C-alkyl radicals which is substituted by one of the abovementioned mono- or di-1-4C-alkylamino radicals. Examples which may be mentioned are the 2-dimethylaminoethyl and 3-dimethylaminopropyl radicals.

Mono- or Di-1-4C-alkylamino-2-4C-alkoxy stands for one of the abovementioned 2-4C-alkoxy radicals which is substituted by one of the abovementioned mono- or di-1-4C-alkylamino radicals. Examples which may be mentioned are the 2-dimethylaminoethoxy and 3-dimethylaminopropoxy radicals.

1-4C-Alkylsulfonyl is a sulfonyl group to which one of the abovementioned 1-4C-alkyl radicals is bonded. An example is the methanesulfonyl radical (CH₃SO₂—).

1-4C-Alkylsulfonylamino is an amino group which is substituted by one of the abovementioned 1-4C-alkylsulfonyl radicals. An example is the methanesulfonylamino radical (CH₃SO₂NH—).

Aryl radicals referred to herein, including those forming part of other groups or radicals, include phenyl or R711-substituted phenyl radicals.

Aryloxy stands for phenoxy or R711-substituted phenoxy.

Aryl-1-4C-alkoxy stands for one of the abovementioned 1-4C-alkoxy radicals, which is substituted by one of the abovementioned aryl radicals. Examples which may be mentioned are the 2-arylethoxy (e.g. phenethoxy) and the arylmethoxy (e.g. benzyloxy) radicals.

Aryloxy-2-4C-alkoxy stands for one of the abovementioned 2-4C-alkoxy radicals, which is substituted by one of the abovementioned aryloxy radicals. An example which may be mentioned is the 2-aryloxyethoxy (e.g. 2-phenoxyethoxy) radical.

Aryloxy-1-4C-alkyl stands for one of the abovementioned 1-4C-alkyl radicals, which is substituted by one of the abovementioned aryloxy radicals. Examples which may be mentioned are the 2-aryloxyethyl (e.g. 2-phenoxyethyl) and the aryloxymethyl (e.g. phenoxymethyl) radicals.

Het1 refers to a 5- to 7-membered saturated heterocyclic ring radical comprising one nitrogen atom, to which R611 and R612 are bound, and, optionally, one further heteroatom selected from a group consisting of nitrogen, oxygen and sulfur, and optionally substituted by R613 on a ring nitrogen atom. Examples for Het2 include e.g. piperidin-1-yl, 4-methyl-piperidin-1-yl, 4-hydroxypiperidin-1-yl, morpholinyl, pyrrolidin-1-yl, piperazin-1-yl, imidazolidin-1-yl, thiomorpholinyl, homopiperidin-1-yl, homopiperazin-1-yl, 4-N-(1-4C-alkyl)-homopiperazin-1-yl or piperazinyl substituted on a ring nitrogen atom by R613 [4-N—(R613)-piperazin-1-yl] such as, for example, 4-N-(1-4C-alkyl)-piperazin-1-yl, 4-N-(hydroxy-2-4C-alkyl)-piperazin-1-yl, 4-N-(dimethylamino-2-4C-alkyl)-piperazin-1-yl, 4-N-(3-6C-cycloalkyl)-piperazin-1-yl, 4-N-formyl-piperazin-1-yl, 4-N-(pyridin-4-yl)-piperazin-1-yl, 4-N-(pyrimidin-2-yl)-piperazin-1-yl or 4-N-(3-6C-cycloalkylmethyl)-piperazin-1-yl.

Het2 refers to a monocyclic or fused bicyclic 5 to 10-membered heteroaryl (heteroaromatic) radical comprising one to three heteroatoms, each of which is selected from a group consisting of nitrogen, oxygen and sulfur, and includes, for example, without being restricted to furanyl, thiophenyl, pyrrolyl, oxazolyl, isoxazolyl, thiazolyl, isothiazolyl, imidazolyl, pyrazolyl, triazolyl, thiadiazolyl, oxadiazolyl, pyridinyl, pyrimidinyl, pyrazinyl, pyridazinyl, benzo-fused analogues thereof, such as, for example, quinazolinyl, quinoxalinyl, cinnolinyl, quinolyl, isoquinolyl, indolyl, isoindolyl, indazolyl, benzothiophenyl, benzofuranyl, benzoxazolyl, benzothiazolyl or benzimidazolyl, or naphthyridinyl, phthalazinyl, imidazopyridinyl, purinyl, pteridinyl or imidazopyridazinyl. The monocyclic 5- to 6-membered radicals, such as, for example, furanyl, thiophenyl, pyrrolyl, pyrimidinyl and pyridinyl, and quinolinyl and indolyl are more worthy to be mentioned. In particular worthy to be mentioned are indolyl, quinolinyl and pyridinyl. In more particular worthy to be mentioned are quinolyl and pyridinyl, especially quinolin-4-yl and, particularly, pyridin-4-yl.

N-(1-4C-alkyl)-piperazinyl stands for the piperazin-1-yl radical substituted by one of the abovementioned 1-4C-alkyl radicals on the 4N ring nitrogen atom.

Naphthyl includes naphthalene-1-yl and naphthalene-2-yl.

The term Het2 includes all the possible isomeric forms thereof, in particular the positional isomers thereof. Thus, e.g. pyridinyl or pyridyl includes pyridin-2-yl, pyridin-3-yl and pyridin-4-yl.

Constituents which are substituted as described herein may be substituted, unless otherwise noted, at any possible position.

The substituents R1, R2 and/or R3 may be attached, unless otherwise noted, at any position of the benzo moiety of the pyrrolodihydroisoquinoline ring.

Suitable salts for compounds of the formula I—depending on substitution—are all acid addition salts or all salts with bases. Particular mention may be made of the pharmacologically tolerable inorganic and organic adds and bases customarily used in pharmacy. Those suitable are, on the one hand, water-insoluble and, particularly, water-soluble acid addition salts with adds such as, for example, hydrochloric add, hydrobromic acid, phosphoric acid, nitric acid, sulphuric acid, acetic acid, citric acid, D-gluconic acid, benzoic acid, 2-(4-hydroxybenzoyl)benzoic acid, butyric add, sulphosalicylic acid, maleic acid, lauric acid, malic acid, fumaric acid, succinic acid, oxalic acid, tartaric acid, embonic add, stearic acid, toluenesulphonic acid, methanesulphonic acid or 3-hydroxy-2-naphthoic acid, the acids being employed in salt preparation—depending on whether a mono- or polybasic acid is concerned and depending on which salt is desired—in an equimolar quantitative ratio or one differing therefrom.

On the other hand, salts with bases are—depending on substitution—also suitable. As examples of salts with bases are mentioned the lithium, sodium, potassium, calcium, aluminium, magnesium, titanium, ammonium, meglumine or guanidinium salts, here, too, the bases being employed in salt preparation in an equimolar quantitative ratio or one differing therefrom.

Pharmacologically intolerable salts, which can be obtained, for example, as process products during the preparation of the compounds according to the invention on an industrial scale, are converted into pharmacologically tolerable salts by processes known to the person skilled in the art.

According to experts knowledge the compounds of the invention as well as their salts may contain, e.g. when isolated in crystalline form, varying amounts of solvents. Included within the scope of the invention are therefore all solvates and in particular all hydrates of the compounds of formula I as well as all solvates and in particular all hydrates of the salts of the compounds of formula I.

Depending on substitution the compounds of formula I can be chiral compounds having, for example, chiral centers and/or chiral axes due to hindered rotation about single bonds. Chiral axes can be present in particular in those compounds according to the invention, in which R7 is a bicyclic ring, or a monocyclic ring substituted in the ortho position with respect to the binding position in which said monocyclic ring is bonded to the pyrrolo[2.1-a]isoquinoline ring system. The invention therefore includes all conceivable pure diastereomers and pure enantiomers and mixtures thereof in any mixing ratio including the racemates. The diastereomer mixtures can be separated into the individual isomers by chromatographic processes. The enantiomers can be separated in a known manner (e.g. by chromatographic processes on chiral phases or by resolution).

In the context of this invention, the term “hyperproliferation” and analogous terms are used to describe aberrant/dysregulated cellular growth, a hallmark of diseases like cancer. This hyperproliferation might be caused by single or multiple cellular/molecular alterations in respective cells and can be, in context of a whole organism, of benign or malignant behaviour. The phrase “inhibition of cell proliferation” is used to denote an ability of the compound to retard the growth of a cell contacted with that compound as compared to cells not contacted with that compound. Most preferable this inhibition of cell proliferation is 100%, meaning that proliferation of all cells is stopped and/or cells undergo programmed cell death. In some preffered embodiments the contacted cell is a neoplastic cell. A neoplastic cell is defined as a cell with aberrant cell proliferation. A benign neoplasia is described by hyperproliferation of cells, incapable of forming an aggressive, metastasizing tumor in-vivo. In contrast, a malignant neoplasia is described by cells with different cellular and biochemical abnormalities, capable of forming tumor metastasis. The aquired functional abnormalities of malignant neoplastic cells (also defined as “hallmarks of cancer”) are replicative potential (“hyperproliferation”), self-sufficiency in growth signals, insensitivity to anti-growth signals, evasion from apoptosis, sustained angiogenesis and tissue invasion and metastasis.

The term “inducer of apoptosis” and analogous terms are used to identify a compound which excecutes programmed cell death in cells contacted with that compound. Apoptosis is defined by complex biochemical events within the contacted cell, such as the activation of cystein specific proteinases (“caspases”) and the fragmentation of chromatin. Induction of apoptosis in cells contacted with the compound might not necessarily coupled with inhibition of cell proliferation. Preferably, the inhibition of cell proliferation and/or induction of apoptosis is specific to cells with aberrant cell growth (hyperproliferation). Thus, compared to cells with aberrant cell growth, normal proliferating or arrested cells are less sensitive or even insensitive to the proliferation inhibiting or apoptosis inducing activity of the compound. Finally, the term “cytotoxic” is used in a more general sense to identify compounds which kill cells by various mechanisms, including the induction of apoptosis/programmed cell death in a cell cycle dependent or cell-cycle independent manner.

A special subaspect (subaspect 1) of aspects a, b, c and d refers to compounds of formula I according to aspects a, b, c and d, in which none of R1, R2 and R3 is bound to the 10-position of the pyrrolo[2.1-a]isoquinoline ring.

Numbering:

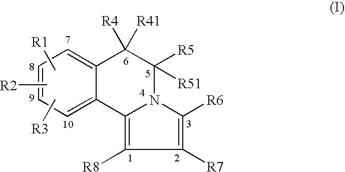

A further special subaspect (subaspect 2) of aspects a, b, c and d refers to compounds of formula I according to aspects a, b, c and d, in which
R8 is phenyl, phenylcarbonyl or —C(O)—N(R82)R83, in which
R82 is hydrogen, 1-4C-alkyl, 3-7C-cycloalkyl, 3-7C-cycloalkyl-1-4C-alkyl, phenyl or phenyl-1-4C-alkyl,
R83 is hydrogen or 1-4C-alkyl, or
R82 and R83 together and with inclusion of the nitrogen atom, to which they are bound, form a heterocyclic ring radical selected from the group consisting of pyrrolidinyl, piperidinyl, morpholinyl or N-(1-4C-alkyl)-piperazinyl.

A further special subaspect (subaspect 3) of aspects a, b, c, and d refers to compounds of formula I according to aspects a, b, c and d, in which.
R1 is halogen, nitro, amino, mono- or di-1-4C-alkylamino, 1-4C-alkyl, 1-4C-alkoxy-2-4C-alkoxy, 3-7C-cycloalkoxy, 3-7C-cycloalkylmethoxy, or completely or predominantly fluorine-substituted 1-4C-alkoxy,
   with the provisio that R1 is not trifluoromethoxy,
R2 is hydrogen, halogen or 1-4C-alkoxy,
R3 is hydrogen or 1-4C-alkoxy, or
R2 and R3 bound to the benzo ring moiety in ortho-position to each other together form a 1-2C-alkylenedioxy bridge, or
R2 and R3 bound to the benzo ring moiety in ortho-position to each other together form a completely or predominantly fluorine-substituted 1-2C-alkylenedioxy bridge, or
R1 and R2 bound to the benzo ring moiety in ortho-position to each other together form a 1-2C-alkylenedioxy bridge and R3 is hydrogen, or
R1 and R2 bound to the benzo ring moiety in ortho-position to each other together form a completely or predominantly fluorine-substituted 1-2C-alkylenedioxy bridge and R3 is hydrogen.

A further special subaspect (subaspect 4) of aspects a, c, d and e refers to compounds of formula I according to aspects a, c and d, in which
R1 is halogen, nitro, amino, mono- or di-1-4C-alkylamino, 1-4C-alkyl, hydroxyl, 1-4C-alkoxy, 1-4C-alkoxy-2-4C-alkoxy, 3-7C-cycloalkoxy, 3-7C-cycloalkylmethoxy, or completely or predominantly fluorine-substituted 1-4C-alkoxy,
R2 is halogen or 1-4C-alkoxy,
R3 is 1-4C-alkoxy, or
R2 and R3 bound to the benzo ring moiety in ortho-position to each other together form a 1-2C-alkylenedioxy bridge, or
R2 and R3 bound to the benzo ring moiety in ortho-position to each other together form a completely or predominantly fluorine-substituted 1-2C-alkylenedioxy bridge.

A further special subaspect (subaspect 5) of aspects a, b, c and d refers to compounds of formula I according to aspects a, b, c and d, in which
R4 is fluorine, chlorine, 1-4C-alkyl, trifluoromethyl, cyclopropyl, cyano, 1-4C-alkoxycarbonyl or —CH₂—O—R411, in which
R411 is hydrogen, 1-4C-alkyl, 1-4C-alkoxy-2-4C-alkyl or 1-4C-alkylcarbonyl,
R41 is hydrogen or 1-4C-alkyl,
R5 is hydrogen,
R51 is hydrogen.

A further special subaspect (subaspect 6) of aspects a, b and d refers to compounds of formula I according to aspects a, b and d, in which
R1 is halogen, nitro, amino, 1-4C-alkyl, 1-4C-alkoxy-2-4C-alkoxy, or completely or predominantly fluorine-substituted 1-4C-alkoxy,
   with the provisio that R1 is not trifluoromethoxy,
R2 is hydrogen or 1-4C-alkoxy,
R3 is hydrogen or 1-4C-alkoxy, or
R1 and R2 bound to the benzo ring moiety in ortho-position to each other together form a completely or predominantly fluorine-substituted 1-2C-alkylenedioxy bridge and R3 is hydrogen.

A further special subaspect (subaspect 7) of said aspects a, c and d refers to compounds of formula I according to aspects a, c and d, in which
R1 is halogen, nitro, amino, 1-4C-alkyl, 1-4C-alkoxy, 1-4C-alkoxy-2-4C-alkoxy, or completely or predominantly fluorine-substituted 1-4C-alkoxy,
R2 is 1-4C-alkoxy,
R3 is 1-4C-alkoxy.

A further special subaspect (subaspect 8) of said aspects a, c and d refers to compounds of formula I according to aspects a, c and d, in which
R1 is halogen, nitro, amino, 1-4C-alkyl, 1-4C-alkoxy, 1-4C-alkoxy-2-4C-alkoxy, or completely or predominantly fluorine-substituted 1-4C-alkoxy,
R2 is halogen,
R3 is 1-4C-alkoxy.

A further special subaspect (subaspect 9) of said aspects a and d refers to compounds of formula I according to aspects a and d, in which
R1 is halogen, nitro, amino, 1-4C-alkyl, 1-4C-alkoxy, 1-4C-alkoxy-2-4C-alkoxy, or completely or predominantly fluorine-substituted 1-4C-alkoxy,
R2 is 1-4C-alkoxy,
R3 is hydrogen.

A further special subaspect (subaspect 10) of said aspects a and d refers to compounds of formula I according to aspects a and d, in which
R1 is 1-4C-alkoxy,
R2 is 1-4C-alkoxy,
R3 is hydrogen.

A further special subaspect (subaspect 11) of said aspects a and d refers to compounds of formula I according to aspects a and d, in which
R1 is halogen or 1-2C-alkoxy,
R2 is hydrogen or 1-2C-alkoxy,
R3 is 1-2C-alkoxy.

A further special subaspect (subaspect 12) of said aspects a, c and d refers to compounds of formula I according to aspects a, c and d, in which
R1 is 1-2C-alkoxy,
R2 is 1-2C-alkoxy,
R3 is 1-2C-alkoxy.

Compounds according to subaspect 12 more worthy to be mentioned are those, in which none of R1, R2 and R3 is bound to the 10-position of the pyrrolo[2.1-a]isoquinoline ring.

A further special subaspect (subaspect 13) of said aspects a and d refers to compounds of formula I according to aspects a and d, in which
R1 is 1-2C-alkoxy,
R2 is hydrogen,
R3 is 1-2C-alkoxy.

Compounds according to subaspect 13 more worthy to be mentioned are those, in which R1 is bound to the 8-position and R3 is bound to the 9-position of the pyrrolo[2.1-a]isoquinoline ring, or those, in which R1 is bound to the 9-position and R3 is bound to the 8-position of the pyrrolo[2.1-a]isoquinoline ring.

A further special subaspect (subaspect 14) of said aspects a, b and d refers to compounds of formula I according to aspects a, b and d, in which
R1 is halogen,
R2 is hydrogen,
R3 is 1-2C-alkoxy,

Compounds according to subaspect 14 more worthy to be mentioned are those, in which R1 is bound to the 8-position and R3 is bound to the 9-position of the pyrrolo[2.1-a]isoquinoline ring, or those, in which R1 is bound to the 9-position and R3 is bound to the 8-position of the pyrrolo[2.1-a]isoquinoline ring.

A further special subaspect (subaspect 15) of said aspects a, b, c and d refers to compounds of formula I according to aspects a, b, c and d, in which
R1 is halogen,
R2 is 1-2C-alkoxy,
R3 is 1-2C-alkoxy.

Compounds according to subaspect 15 more worthy to be mentioned are those, in which none of R1, R2 and R3 is bound to the 10-position of the pyrrolo[2.1-a]isoquinoline ring.

A further special subaspect (subaspect 16) of said aspects a, b, c and d refers to compounds of formula I according to aspects a, b, c and d, in which
R1 is halogen,
R2 is halogen,
R3 is 1-2C-alkoxy.

Compounds according to subaspect 16 more worthy to be mentioned are those, in which none of R1, R2 and R3 is bound to the 10-position of the pyrrolo[2.1-a]isoquinoline ring.

A further special subaspect (subaspect 17) of said aspects a, b, c and d refers to compounds of formula I according to aspects a, b, c and d, in which
R1 is halogen, nitro, 1-4C-alkyl or 1-4C-alkoxy-2-4C-alkoxy.

A further special subaspect (subaspect 18) of aspects a, b, c and d refers to compounds of formula I according to aspects a, b, c and d, in which
R1 is chlorine or fluorine.

Compounds according to subaspect 18 more worthy to be mentioned are those, in which R1 is not bound to the 10-position of the pyrrolo[2.1-a]isoquinoline ring.

A further special subaspect (subaspect 19) of aspects a, b, c and d refers to compounds of formula I according to aspects a, b, c and d, in which
R4 is 1-4C-alkyl,
R41 is hydrogen or 1-4C-alkyl,
R5 is hydrogen,
R51 is hydrogen.

A further special subaspect (subaspect 20) of aspects a, b, c and d refers to compounds of formula I according to aspects a, b, c and d, in which
R4 is 1-4C-alkyl,
R41 is hydrogen or 1-4C-alkyl,
R5 is hydrogen,
R51 is hydrogen.

A further special subaspect (subaspect 21) of aspects a, b, c and d refers to compounds of formula I according to aspects a, b, c and d, in which either
R4 is 1-4C-alkyl, or
R41 is 1-4C-alkyl.

A further special subaspect (subaspect 22) of aspects a, b, c and d refers to compounds of formula I according to aspects a, b, c and d, in which
R6 is 1-6C-alkyl, or 1-4C-alkyl substituted by R61, in which
R61 is 1-4C-alkoxycarbonyl.

A further special subaspect (subaspect 23) of aspects a, b, c and d refers to compounds of formula I according to aspects a, b, c and d, in which
R6 is methyl, ethyl or methoxycarbonylethyl.

A further special subaspect (subaspect 24) of aspects a, b, c and d refers to compounds of formula I according to aspects a, b, c and d, in which
R6 is methyl.

A further special subaspect (subaspect 25) of aspects a, b, c and d refers to compounds of formula I according to aspects a, b, c and d, in which
R6 is methoxycarbonylethyl.

A further special subaspect (subaspect 26) of aspects a, b, c and d refers to compounds of formula I according to aspects a, b, c and d, in which
R7 is Het2, R74- and/or R75-substituted Het2, or hydroxy-dimethyl-phenyl, in which
Het2 is pyridinyl or quinolinyl,
R74 is halogen, 1-4C-alkyl, trifluoromethyl, 1-4C-alkoxy, cyano, amino, mono- or di-1-4C-alkylamino, 1-4C-alkoxycarbonyl, carboxyl, nitro, phenyl or phenyloxy,
R75 is 1-4C-alkyl.

Compounds according to subaspect 26 more worthy to be mentioned are those, in which
R7 is Het2, R74 and/or R75-substituted Het2, or 4-hydroxy-3,5-dimethylphenyl, in which
Het2 is pyridinyl or quinolinyl,
R74 is halogen, 1-4C-alkyl, trifluoromethyl, 1-4C-alkoxy, cyano, amino, mono- or di-1-4C-alkylamino, 1-4C-alkoxycarbonyl, carboxyl, nitro, phenyl or phenyloxy,
R75 is 1-4C-alkyl.

A further special subaspect (subaspect 27) of aspects a, b, c and d refers to compounds of formula I according to aspects a, b, c and d, in which
R7 is pyridin-4-yl.

A further special subaspect (subaspect 28) of aspects a, b, c and d refers to compounds of formula I according to aspects a, b, c and d, in which
R7 is 2,6-dimethylpyridin-4-yl.

A further special subaspect (subaspect 29) of aspects a, b, c and d refers to compounds of formula I according to aspects a, b, c and d, in which
R7 is quinolin-4-yl.

A further special subaspect (subaspect 30) of aspects a, b, c and d refers to compounds of formula I according to aspects a, b, c and d, in which
R8 is phenyl, phenylcarbonyl or —C(O)—N(R82)R83, in which
R82 is hydrogen, 1-4C-alkyl, 3-7C-cycloalkyl, 3-7C-cycloalkyl-1-4C-alkyl, phenyl or phenyl-1-4C-alkyl,
R83 is hydrogen or 1-4C-alkyl, or
R82 and R83 together and with inclusion of the nitrogen atom, to which they are bound, form a heterocyclic ring radical selected from the group consisting of pyrrolidinyl, piperidinyl, morpholinyl or N-1-4C-alkyl)-piperazinyl.

A further special subaspect (subaspect 31) of aspects a, b, c and d refers to compounds of formula I according to aspects a, b, c and d, in which
R8 is phenyl, phenylcarbonyl or —C(O)—N(R82)R83, in which
R82 is hydrogen, 1-4C-alkyl, 3-7C-cycloalkyl or phenyl,
R83 is hydrogen or 1-4C-alkyl, or
R82 and R83 together and with inclusion of the nitrogen atom, to which they are bound, form a pyrrolidinyl ring.

Special subaspects more worthy to be mentioned are the subaspects 11, 12, 15, 23, 24, 25, 26, 27, 28 and 29.

Compounds according to aspect a more worthy to be mentioned are those of formula I, in which
R1 is halogen, nitro, amino, 1-4C-alkyl, 1-4C-alkoxy, 1-4C-alkoxy-2-4C-alkoxy, or completely or predominantly fluorine-substituted 1-4C-alkoxy,
R2 is hydrogen or 1-4C-alkoxy,
R3 is hydrogen or 1-4C-alkoxy, or
R1 and R2 bound to the benzo ring moiety in ortho-position to each other together form a completely or predominantly fluorine-substituted 1-2C-alkylenedioxy bridge and R3 is hydrogen,
R4 is hydrogen or 1-4C-alkyl,
R41 is hydrogen or 1-4C-alkyl,
R5 is hydrogen,
R51 is hydrogen,
   or
R4 is hydrogen,
R41 is hydrogen,
R5 is hydrogen,
R51 is hydrogen,
R6 is 1-6C-alkyl or 1-4C-alkyl substituted by R61, in which
R61 is 1-4C-alkoxycarbonyl,
R7 is phenyl, Het2, R71- and/or R72- and/or R73-substituted phenyl, R74- and/or R75-substituted Het2, or naphthyl, in which
Het2 is a heteroaryl radical selected from the group consisting of furanyl, thiophenyl, pyrrolyl, pyridinyl, quinolyl, indolyl, benzothiophenyl and benzofuranyl,
R71 is hydroxyl, halogen, nitro, trifluoromethyl, 1-4C-alkyl, 1-4C-alkoxy, amino, mono- or di-1-4C-alkylamino, 1-4C-alkylsulphonylamino, tolylsulphonylamino or aryloxy, in which
aryl is R711-substituted phenyl, in which
R711 is halogen,
R72 is 1-4C-alkyl, 1-4C-alkoxy or 1-4C-alkoxycarbonyl,
R73 is 1-4C-alkyl or 1-4C-alkoxy,
R74 is 1-4C-alkyl, trifluoromethyl, 1-4C-alkoxy, 1-4C-alkoxycarbonyl, nitro, phenyl or phenyloxy,
R75 is 1-4C-alkyl,
R8 is phenyl, phenylcarbonyl or —C(O)—N(R82)R83, in which
R82 is hydrogen, 1-4C-alkyl, 3-7C-cycloalkyl or phenyl,
R83 is hydrogen or 1-alkyl, or
R82 and R83 together and with inclusion of the nitrogen atom, to which they are bound, form a heterocyclic ring radical selected from the group consisting of pyrrolidinyl and piperidinyl,
   and the salts, stereoisomers, hydrates and hydrates of the salts of these compounds.

Compounds according to aspect a further more worthy to be mentioned are those of formula I, in

which

R1 is halogen, nitro, amino, 1-4C-alkyl, 1-4C-alkoxy, 1-4C-alkoxy-2-4C-alkoxy, or completely or predominantly fluorine-substituted 1-4C-alkoxy,
R2 is 1-4C-alkoxy,
R3 is hydrogen,
   and none of R1, R2 and R3 is bound to the 10-position of the pyrrolo[2.1-a]isoquinoline ring, in particular, none of R1 and R2 is bound to the 7- or 10-position of the pyrrolo[2.1-a]isoquinoline ring,
R4 is hydrogen,
R41 is hydrogen,
R5 is hydrogen,
R51 is hydrogen,
R6 is 1-6C-alkyl, formyl, or 1-4C-alkyl substituted by R61, in which
R61 is 1-4C-alkoxycarbonyl,
R7 is phenyl, Het2, R71- and/or R72- and/or R73-substituted phenyl, R74- and/or R75-substituted Het2, or naphthyl, in which
Het2 is a heteroaryl radical selected from the group consisting of furanyl, thiophenyl, pyrrolyl, pyridinyl, quinolyl, indolyl, benzothiophenyl and benzofuranyl,
R71 is hydroxyl, 1-4C-alkoxy, amino or mono- or di-1-4C-alkylamino,
R72 is 1-4C-alkyl or 1-4C-alkoxy,
R73 is 1-4C-alkyl or 1-4C-alkoxy,
R74 is 1-4C-alkyl, trifluoromethyl, 1-4C-alkoxy, 1-4C-alkoxycarbonyl, nitro, phenyl or phenyloxy,
R75 is 1-4C-alkyl,
R8 is phenyl, phenylcarbonyl, or —C(O)—N(R82)R83, in which
R82 is hydrogen, 1-4C-alkyl, 3-7C-cycloalkyl or phenyl,
R83 is hydrogen or 1-4C-alkyl, or
R82 and R83 together and with inclusion of the nitrogen atom, to which they are bound, form a heterocyclic ring radical selected from the group consisting of pyrrolidinyl and piperidinyl,
   and the salts, stereoisomers, hydrates and hydrates of the salts of these compounds.

Yet compounds according to aspect a further more worthy to be mentioned are those of formula I, in

which

R1 is halogen, nitro, amino, 1-4C-alkyl, 1-4C-alkoxy, 1-4C-alkoxy-2-4C-alkoxy, or completely or predominantly fluorine-substituted 1-4C-alkoxy,
R2 is 1-4C-alkoxy,
R3 is hydrogen,
   and none of R1, R2 and R3 is bound to the 10-position of the pyrrolo[2.1-a]isoquinoline ring, in particular, none of R1 and R2 is bound to the 7- or 10-position of the pyrrolo[2.1-a]isoquinoline ring,
R4 is 1-4C-alkyl,
R41 is hydrogen or 1-4C-alkyl,
R5 is hydrogen,
R51 is hydrogen,
R6 is 1-6C-alkyl, formyl, or 1-4C-alkyl substituted by R61, in which
R61 is 1-4C-alkoxycarbonyl,
R7 is phenyl, Het2, R71- and/or R72- and/or R73-substituted phenyl, R74- and/or R75-substituted Het2, or naphthyl, in which
Het2 is a heteroaryl radical selected from the group consisting of furanyl, thiophenyl, pyrrolyl, pyridinyl, quinolyl, indolyl, benzothiophenyl and benzofuranyl,
R71 is hydroxyl, 1-4C-alkoxy, amino or mono- or di-1-4C-alkylamino,
R72 is 1-4C-alkyl or 1-4C-alkoxy,
R73 is 1-4C-alkyl or 1-4C-alkoxy,
R74 is 1-4C-alkyl, trifluoromethyl, 1-4C-alkoxy, 1-4C-alkoxycarbonyl, nitro, phenyl or phenyloxy,
R75 is 1-4C-alkyl,
R8 is phenyl, phenylcarbonyl, or —C(O)—N(R82)R83, in which
R82 is hydrogen, 1-4C-alkyl, 3-7C-cycloalkyl or phenyl,
R83 is hydrogen or 1-4C-alkyl, or
R82 and R83 together and with inclusion of the nitrogen atom, to which they are bound, form a heterocyclic ring radical selected from the group consisting of pyrrolidinyl and piperidinyl,
   and the salts, stereoisomers, hydrates and hydrates of the salts of these compounds.

Compounds according to aspect a in particular worthy to be mentioned are those of formula I, in which
R1 is chlorine, fluorine, nitro, amino, methyl, methoxy, methoxyethoxy or difluoromethoxy,
R2 is hydrogen or methoxy,
R3 is hydrogen or methoxy, or
R1 and R2 bound to the benzo ring moiety in ortho-position to each other together form a difluoromethylenedioxy bridge and R3 is hydrogen,
   and none of R1, R2 and R3 is bound to the 10-position of the pyrrolo[2.1-a]isoquinoline ring,
R4 is hydrogen or methyl,
R41 is hydrogen or methyl,
R5 is hydrogen,
R51 is hydrogen,
   or
R4 is hydrogen,
R41 is hydrogen,
R5 is hydrogen,
R51 is hydrogen,
R6 is methyl, ethyl or methoxycarbonylethyl,
R7 is phenyl, Het2, R71- and/or R72- and/or R73-substituted phenyl, or naphthyl, in which
Het2 is indolyl, pyridinyl or quinolyl,
R71 is hydroxyl, chlorine, methoxy, dimethylamino, or aryloxy, in which
aryl is R711-substituted phenyl, in which
R711 is chlorine,
R72 is methyl, tert-butyl or methoxy,
R73 is methyl, tert-butyl or methoxy,
R8 is phenyl, phenylcarbonyl or —C(O)—N(R82)R83, in which
R82 is hydrogen, methyl, ethyl, iso-propyl, iso-butyl, cyclohexyl, cyclopropyl or phenyl,
R83 is hydrogen or methyl, or
R82 and R83 together and with inclusion of the nitrogen atom, to which they are bound, form a pyrrolidinyl radical,
   and the salts, stereoisomers, hydrates and hydrates of the salts of these compounds.

Compounds according to aspect a in further particular worthy to be mentioned are those of formula I, in which

either

R1 is bonded in the 8-position of the pyrrolodihydroisoquinoline scaffold, and is chlorine, methoxy, methoxyethoxy or difluoromethoxy,
R2 is bonded in the 9-position of the pyrrolodihydroisoquinoline scaffold, and is methoxy,
R3 is hydrogen,
   or
R1 is bonded in the 9-position of the pyrrolodihydroisoquinoline scaffold, and is chlorine, fluorine, methoxy, nitro, methyl, amino, or difluoromethoxy,
R2 is bonded in the 8-position of the pyrrolodihydroisoquinoline scaffold, and is methoxy,
R3 is hydrogen,
R4 is hydrogen,
R41 is hydrogen,
R5 is hydrogen,
R51 is hydrogen,
R6 is methyl, ethyl or methoxycarbonylethyl,
R7 is phenyl, Het2, R71- and/or R72- and/or R73-substituted phenyl, or naphthyl, in which
Het2 is indolyl, pyridinyl or quinolyl,
R71 is hydroxyl, methoxy or dimethylamino, in which
R72 is methyl, tert-butyl or methoxy,
R73 is methyl, tert-butyl or methoxy,
R8 is phenylcarbonyl, or —C(O)—N(R82)R83, in which
R82 is hydrogen, methyl, ethyl, iso-propyl, iso-butyl, cyclohexyl, cyclopropyl or phenyl,
R83 is hydrogen or methyl, or
R82 and R83 together and with inclusion of the nitrogen atom, to which they are bound, form a pyrrolidinyl radical,
   and the salts, stereoisomers, hydrates and hydrates of the salts of these compounds.

Yet compounds according to aspect a in further particular worthy to be mentioned are those of formula I, in which

either

R1 is bonded in the 8-position of the pyrrolodihydroisoquinoline scaffold, and is chlorine, methoxy, methoxyethoxy or difluoromethoxy,
R2 is bonded in the 9-position of the pyrrolodihydroisoquinoline scaffold, and is methoxy,
R3 is hydrogen,
   or
R1 is bonded in the 9-position of the pyrrolodihydroisoquinoline scaffold, and is chlorine, fluorine, methoxy, nitro, methyl, amino, or difluoromethoxy,
R2 is bonded in the 8-position of the pyrrolodihydroisoquinoline scaffold, and is methoxy,
R3 is hydrogen,
R4 is methyl,
R41 is hydrogen or methyl,
R5 is hydrogen,
R51 is hydrogen,
R6 is methyl, ethyl or methoxycarbonylethyl,
R7 is phenyl, Het2, R71- and/or R72- and/or R73-substituted phenyl, or naphthyl, in which
Het2 is indolyl, pyridinyl or quinolyl,
R71 is hydroxyl, methoxy or dimethylamino, in which
R72 is methyl, tert-butyl or methoxy,
R73 is methyl, tert-butyl or methoxy,
R8 is phenylcarbonyl, or —C(O)—N(R82)R83, in which
R82 is hydrogen, methyl, ethyl, iso-propyl, iso-butyl, cyclohexyl, cyclopropyl or phenyl,
R83 is hydrogen or methyl, or
R82 and R83 together and with inclusion of the nitrogen atom, to which they are bound, form a pyrrolidinyl radical,
   and the salts, stereoisomers, hydrates and hydrates of the salts of these compounds.

Compounds according to aspect b more worthy to be mentioned are those of formula I, in which
R1 is halogen, nitro, amino, 1-4C-alkyl, 1-4C-alkoxy-2-4C-alkoxy, or completely or predominantly fluorine-substituted 1-4C-alkoxy,
   with the provisio that R1 is not trifluoromethoxy,
R2 is hydrogen or 1-4C-alkoxy,
R3 is hydrogen or 1-4C-alkoxy, or
R1 and R2 bound to the benzo ring moiety in ortho-position to each other together form a completely or predominantly fluorine-substituted 1-2C-alkylenedioxy bridge and R3 is hydrogen,
R4 is hydrogen or 1-4C-alkyl,
R41 is hydrogen or 1-4C-alkyl,
R5 is hydrogen,
R51 is hydrogen,
   or
R4 is hydrogen,
R41 is hydrogen,
R5 is hydrogen,
R51 is hydrogen,
R6 is 1-4C-alkyl, formyl, or 1-4C-alkyl substituted by R61, in which
R61 is 1-4C-alkoxycarbonyl,
R7 is phenyl, Het2, R71- and/or R72- and/or R73-substituted phenyl, R74- and/or R75-substituted Het2, or naphthyl, in which
Het2 is a heteroaryl radical selected from the group consisting of furanyl, thiophenyl, pyrrolyl, pyridinyl, quinolyl, indolyl, benzothiophenyl and benzofuranyl,
R71 is hydroxyl, halogen, nitro, trifluoromethyl, 1-4C-alkyl, 1-4C-alkoxy, amino, mono- or di-1-4C-alkylamino, 1-4C-alkylsulphonylamino, tolylsulphonylamino or aryloxy, in which
aryl is R711-substituted phenyl, in which
R711 is halogen,
R72 is 1-4C-alkyl, 1-4C-alkoxy or 1-4C-alkoxycarbonyl,
R73 is 1-4C-alkyl or 1-4C-alkoxy,
R74 is 1-4C-alkyl, trifluoromethyl, 1-4C-alkoxy, 1-4C-alkoxycarbonyl, nitro, phenyl or phenyloxy,
R75 is 1-4C-alkyl,
R8 is phenyl, phenylcarbonyl, —C(O)—N(R82)R83 or —C(O)—OR9, in which
R82 is hydrogen, 1-4C-alkyl, 3-7C-cycloalkyl or phenyl,
R83 is hydrogen or 1-4C-alkyl, or
R82 and R83 together and with inclusion of the nitrogen atom, to which they are bound, form a heterocyclic ring radical selected from the group consisting of pyrrolidinyl and piperidinyl,
R9 is 1-4C-alkyl,
   and the salts, stereoisomers, hydrates and hydrates of the salts of these compounds.

Compounds according to aspect b further more worthy to be mentioned are those of formula I, in

which

R1 is halogen, nitro, amino, 1-4C-alkyl, 1-4C-alkoxy-2-4C-alkoxy, or completely or predominantly fluorine-substituted 1-4C-alkoxy,
   with the provisio that R1 is not trifluoromethoxy,
R2 is 1-4C-alkoxy,
R3 is hydrogen, or
R1 and R2 bound to the benzo ring moiety in ortho-position to each other together form a completely or predominantly fluorine-substituted 1-2C-alkylenedioxy bridge and R3 is hydrogen,
   and none of R1, R2 and R3 is bound to the 10-position of the pyrrolo[2.1-a]isoquinoline ring, in particular, none of R1 and R2 is bound to the 7- or 10-position of the pyrrolo[2.1-a]isoquinoline ring,
R4 is hydrogen,
R41 is hydrogen,
R5 is hydrogen,
R51 is hydrogen,
R6 is 1-6C-alkyl, formyl, or 1-4C-alkyl substituted by R61, in which
R61 is 1-4C-alkoxycarbonyl,
R7 is phenyl, Het2, R71- and/or R72- and/or R73-substituted phenyl, R74- and/or R75-substituted Het2, or naphthyl, in which
Het2 is a heteroaryl radical selected from the group consisting of furanyl, thiophenyl, pyrrolyl, pyridinyl, quinolyl, indolyl, benzothiophenyl and benzofuranyl,
R71 is hydroxyl, 1-4C-alkoxy, amino or mono- or di-1-4C-alkylamino,
R72 is 1-4C-alkyl or 1-4C-alkoxy,
R73 is 1-4C-alkyl or 1-4C-alkoxy,
R74 is 1-4C-alkyl, trifluoromethyl, 1-4C-alkoxy, 1-4C-alkoxycarbonyl, nitro, phenyl or phenyloxy,
R75 is 1-4C-alkyl,
R8 is phenyl, phenylcarbonyl, —C(O)—N(R82)R83 or, in particular, —C(O)—OR9, in which
R82 is hydrogen, 1-4C-alkyl, 3-7C-cycloalkyl or phenyl,
R83 is hydrogen or 1-4C-alkyl, or
R82 and R83 together and with inclusion of the nitrogen atom, to which they are bound, form a heterocyclic ring radical selected from the group consisting of pyrrolidinyl and piperidinyl,
R9 is 1-4C-alkyl,
   and the salts, stereoisomers, hydrates and hydrates of the salts of these compounds.

Yet compounds according to aspect b further more worthy to be mentioned are those of formula I, in

which

R1 is halogen, nitro, amino, 1-4C-alkyl, 1-4C-alkoxy-2-4C-alkoxy, or completely or predominantly fluorine-substituted 1-4C-alkoxy,
   with the provisio that R1 is not trifluoromethoxy,
R2 is 1-4C-alkoxy,
R3 is hydrogen, or
R1 and R2 bound to the benzo ring moiety in ortho-position to each other together form a completely or predominantly fluorine-substituted 1-2C-alkylenedioxy bridge and R3 is hydrogen,
   and none of R1, R2 and R3 is bound to the 10-position of the pyrrolo[2.1-a]isoquinoline ring, in particular, none of R1 and R2 is bound to the 7- or 10-position of the pyrrolo[2.1-a]isoquinoline ring,
R4 is 1-4C-alkyl,
R41 is hydrogen or 1-4C-alkyl,
R5 is hydrogen,
R51 is hydrogen,
R6 is 1-6C-alkyl, formyl, or 1-4C-alkyl substituted by R61, in which
R61 is 1-4C-alkoxycarbonyl,
R7 is phenyl, Het2, R71- and/or R72- and/or R73-substituted phenyl, R74- and/or R75-substituted Het2, or naphthyl, in which
Het2 is a heteroaryl radical selected from the group consisting of furanyl, thiophenyl, pyrrolyl, pyridinyl, quinolyl, indolyl, benzothiophenyl and benzofuranyl,
R71 is hydroxyl, 1-4C-alkoxy, amino or mono- or di-1-4C-alkylamino,
R72 is 1-4C-alkyl or 1-4C-alkoxy,
R73 is 1-4C-alkyl or 1-4C-alkoxy,
R74 is 1-4C-alkyl, trifluoromethyl, 1-4C-alkoxy, 1-4C-alkoxycarbonyl, nitro, phenyl or phenyloxy,
R75 is 1-4C-alkyl,
R8 is phenyl, phenylcarbonyl, —C(O)—N(R82)R83 or, in particular, —C(O)—OR9, in which
R82 is hydrogen, 1-4C-alkyl, 3-7C-cycloalkyl or phenyl,
R83 is hydrogen or 1-4C-alkyl, or
R82 and R83 together and with inclusion of the nitrogen atom, to which they are bound, form a heterocyclic ring radical selected from the group consisting of pyrrolidinyl and piperidinyl,
R9 is 1-4C-alkyl,
   and the salts, stereoisomers, hydrates and hydrates of the salts of these compounds.

Compounds according to aspect b in particular worthy to be mentioned are those of formula I, in which
R1 is chlorine, fluorine, nitro, amino, methyl, methoxyethoxy or difluoromethoxy,
R2 is hydrogen or methoxy,
R3 is hydrogen or methoxy, or
R1 and R2 bound to the benzo ring moiety in ortho-position to each other together form a difluoromethylenedioxy bridge and R3 is hydrogen,
   and none of R1, R2 and R3 is bound to the 10-position of the pyrrolo[2.1-a]isoquinoline ring,
R4 is hydrogen or methyl,
R41 is hydrogen or methyl,
R5 is hydrogen,
R51 is hydrogen,
   or
R4 is hydrogen,
R41 is hydrogen,
R5 is hydrogen,
R51 is hydrogen,
R6 is methyl, ethyl or methoxycarbonylethyl,
R7 is phenyl, Het2, R71- and/or R72- and/or R73-substituted phenyl, or naphthyl, in which
Het2 is indolyl, pyridinyl or quinolyl,
R71 is hydroxyl, chlorine, methoxy, dimethylamino, or aryloxy, in which
aryl is R711-substituted phenyl, in which
R711 is chlorine,
R72 is methyl, tert-butyl or methoxy,
R73 is methyl, tert-butyl or methoxy,
R8 is phenyl, phenylcarbonyl, —C(O)—N(R82)R83 or —C(O)—OR9, in which
R82 is hydrogen, methyl, ethyl, iso-propyl, iso-butyl, cyclohexyl, cyclopropyl or phenyl,
R83 is hydrogen or methyl, or
R82 and R83 together and with inclusion of the nitrogen atom, to which they are bound, form a pyrrolidinyl radical,
R9 is methyl or ethyl,
   and the salts, stereoisomers, hydrates and hydrates of the salts of these compounds.

Compounds according to aspect b in further particular worthy to be mentioned are those of formula I, in which

either

R1 is bonded in the 8-position of the pyrrolodihydroisoquinoline scaffold, and is chlorine, methoxyethoxy or difluoromethoxy,
R2 is bonded in the 9-position of the pyrrolodihydroisoquinoline scaffold, and is methoxy,
R3 is hydrogen,
   or
R1 is bonded in the 9-position of the pyrrolodihydroisoquinoline scaffold, and is chlorine, fluorine, nitro, methyl, amino, or difluoromethoxy,
R2 is bonded in the 8-position of the pyrrolodihydroisoquinoline scaffold, and is methoxy,
R3 is hydrogen,
   or
R1 and R2 bound to the benzo ring moiety in ortho-position to each other together form a difluoromethylenedioxy bridge and R3 is hydrogen,
R4 is hydrogen,
R41 is hydrogen,
R5 is hydrogen,
R61 is hydrogen,
R6 is methyl, ethyl or methoxycarbonylethyl,
R7 is phenyl, Het2, R71- and/or R72- and/or R73-substituted phenyl, or naphthyl, in which
Het2 is indolyl, pyridinyl or quinolyl,
R71 is hydroxyl, methoxy or dimethylamino, in which
R72 is methyl, tert-butyl or methoxy,
R73 is methyl, tertbutyl or methoxy,
R8 is phenylcarbonyl, —C(O)—N(R82)R83 or, in particular, —C(O)—OR9, in which
R82 is hydrogen, methyl, ethyl, iso-propyl, iso-butyl, cyclohexyl, cyclopropyl or phenyl,
R83 is hydrogen or methyl, or
R82 and R83 together and with inclusion of the nitrogen atom, to which they are bound, form a pyrrolidinyl radical,
R9 is methyl or ethyl,
   and the salts, stereoisomers, hydrates and hydrates of the salts of these compounds.

Yet compounds according to aspect b in further particular worthy to be mentioned are those of formula I, in which

either

R1 is bonded in the 8-position of the pyrrolodihydroisoquinoline scaffold, and is chlorine, methoxyethoxy or difluoromethoxy,
R2 is bonded in the 9-position of the pyrrolodihydroisoquinoline scaffold, and is methoxy,
R3 is hydrogen,
   or
R1 is bonded in the 9-position of the pyrrolodihydroisoquinoline scaffold, and is chlorine, fluorine, nitro, methyl, amino, or difluoromethoxy,
R2 is bonded in the 8-position of the pyrrolodihydroisoquinoline scaffold, and is methoxy,
R3 is hydrogen,
   or
R1 and R2 bound to the benzo ring moiety in ortho-position to each other together form a difluoromethylenedioxy bridge and R3 is hydrogen,
R4 is methyl,
R41 is hydrogen or methyl,
R5 is hydrogen,
R51 is hydrogen,
R6 is methyl, ethyl or methoxycarbonylethyl,
R7 is phenyl, Het2, R71- and/or R72- and/or R73-substituted phenyl, or naphthyl, in which
Het2 is indolyl, pyridinyl or quinolyl,
R71 is hydroxyl, methoxy or dimethylamino, in which
R72 is methyl, tert-butyl or methoxy,
R73 is methyl, tert-butyl or methoxy,
R8 is phenylcarbonyl, —C(O)—N(R82)R83 or, in particular, —C(O)—OR9, in which
R82 is hydrogen, methyl, ethyl, iso-propyl, iso-butyl, cyclohexyl, cyclopropyl or phenyl,
R83 is hydrogen or methyl, or
R82 and R83 together and with inclusion of the nitrogen atom, to which they are bound, form a pyrrolidinyl radical,
R9 is methyl or ethyl,
   and the salts, stereoisomers, hydrates and hydrates of the salts of these compounds.

Compounds according to aspect c more worthy to be mentioned are those of formula I, in which
R1 is halogen, nitro, amino, 1-4C-alkyl, 1-4C-alkoxy, 1-4C-alkoxy-2-4C-alkoxy, or completely or predominantly fluorine-substituted 1-4C-alkoxy,
R2 is 1-4C-alkoxy,
R3 is 1-4C-alkoxy,
R4 is hydrogen or 1-4C-alkyl,
R41 is hydrogen or 1-4C-alkyl,
R5 is hydrogen,
R51 is hydrogen,
   or
R4 is hydrogen,
R41 is hydrogen,
R5 is hydrogen,
R51 is hydrogen,
R6 is 1-6C-alkyl or 1-4C-alkyl substituted by R61, in which
R61 is 1-4C-alkoxycarbonyl,
R7 is phenyl, Het2, R71- and/or R72- and/or R73-substituted phenyl, R74- and/or R75-substituted Het2, or naphthyl, in which
Het2 is a heteroaryl radical selected from the group consisting of furanyl, thiophenyl, pyrrolyl, pyridinyl, quinolyl, indolyl, benzothiophenyl and benzofuranyl,
R71 is hydroxyl, halogen, nitro, trifluoromethyl, 1-4C-alkyl, 1-4C-alkoxy, amino, mono- or di-1-4C-alkylamino, 1-4C-alkylsulphonylamino, tolylsulphonylamino or aryloxy, in which
aryl is R711-substituted phenyl, in which
R711 is halogen,
R72 is 1-4C-alkyl, 1-4C-alkoxy or 1-4C-alkoxycarbonyl,
R73 is 1-4C-alkyl or 1-4C-alkoxy,
R74 is 1-4C-alkyl, trifluoromethyl, 1-4C-alkoxy, 1-4C-alkoxycarbonyl, nitro, phenyl or phenyloxy,
R75 is 1-4C-alkyl,
R8 is phenyl, phenylcarbonyl, —C(O)—N(R82)R83 or —C(O)—OR9, in which
R82 is hydrogen, 1-4C-alkyl, 3-7C-cycloalkyl or phenyl,
R83 is hydrogen or 1-4C-alkyl, or
R82 and R83 together and with inclusion of the nitrogen atom, to which they are bound, form a heterocyclic ring radical selected from the group consisting of pyrrolidinyl and piperidinyl,
R9 is 1-4C-alkyl,
   and the salts, stereoisomers, hydrates and hydrates of the salts of these compounds.

Compounds according to aspect c further more worthy to be mentioned are those of formula I, in

which

R1 is halogen, nitro, amino, 1-4C-alkyl, 1-4C-alkoxy, 1-4C-alkoxy-2-4C-alkoxy, or completely or predominantly fluorine-substituted 1-4C-alkoxy,
R2 is 1-4C-alkoxy,
R3 is 1-4C-alkoxy, and none of R1, R2 and R3 is bound to the 10-position of the pyrrolo[2.1-a]isoquinoline ring,
R4 is hydrogen,
R41 is hydrogen,
R5 is hydrogen,
R51 is hydrogen,
R6 is 1-6C-alkyl, formyl, or 1-4C-alkyl substituted by R61, in which
R61 is 1-4C-alkoxycarbonyl,
R7 is phenyl, Het2, R71- and/or R72- and/or R73-substituted phenyl, R74- and/or R75-substituted Het2, or naphthyl, in which
Het2 is a heteroaryl radical selected from the group consisting of furanyl, thiophenyl, pyrrolyl, pyridinyl, quinolyl, indolyl, benzothiophenyl and benzofuranyl,
R71 is hydroxyl, 1-4C-alkoxy, amino or mono- or di-1-4C-alkylamino,
R72 is 1-4C-alkyl or 1-4C-alkoxy,
R73 is 1-4C-alkyl or 1-4C-alkoxy,
R74 is 1-4C-alkyl, trifluoromethyl, 1-4C-alkoxy, 1-4C-alkoxycarbonyl, nitro, phenyl or phenyloxy,
R75 is 1-4C-alkyl,
R8 is phenyl, phenylcarbonyl, —C(O)—N(R82)R83 or, in particular, —C(O)—OR9, in which
R82 is hydrogen, 1-4C-alkyl, 3-7C-cycloalkyl or phenyl,
R83 is hydrogen or 1-4C-alkyl, or
R82 and R83 together and with inclusion of the nitrogen atom, to which they are bound, form a heterocyclic ring radical selected from the group consisting of pyrrolidinyl and piperidinyl,
R9 is 1-4C-alkyl,
   and the salts, stereoisomers, hydrates and hydrates of the salts of these compounds.

Yet compounds according to aspect c further more worthy to be mentioned are those of formula I, in

which

R1 is halogen, nitro, amino, 1-4C-alkyl, 1-4C-alkoxy, 1-4C-alkoxy-2-4C-alkoxy, or completely or predominantly fluorine-substituted 1-4C-alkoxy,
R2 is 1-4C-alkoxy,
R3 is 1-4C-alkoxy,
   and none of R1, R2 and R3 is bound to the 10-position of the pyrrolo[2.1-a]isoquinoline ring,
R4 is 1-4C-alkyl,
R41 is hydrogen or 1-4C-alkyl,
R5 is hydrogen,
R51 is hydrogen,
R6 is 1-6C-alkyl, formyl, or 1-4C-alkyl substituted by R61, in which
R61 is 1-4C-alkoxycarbonyl,
R7 is phenyl, Het2, R71- and/or R72- and/or R73-substituted phenyl, R74- and/or R75-substituted Het2, or naphthyl, in which
Het2 is a heteroaryl radical selected from the group consisting of furanyl, thiophenyl, pyrrolyl, pyridinyl, quinolyl, indolyl, benzothiophenyl and benzofuranyl,
R71 is hydroxyl, 1-4C-alkoxy, amino or mono- or di-1-4C-alkylamino,
R72 is 1-4C-alkyl or 1-4C-alkoxy,
R73 is 1-4C-alkyl or 1-4C-alkoxy,
R74 is 1-4C-alkyl, trifluoromethyl, 1-4C-alkoxy, 1-4C-alkoxycarbonyl, nitro, phenyl or phenyloxy,
R75 is 1-4C-alkyl,
R8 is phenyl, phenylcarbonyl, —C(O)—N(R82)R83 or, in particular, —C(O)—OR9, in which
R82 is hydrogen, 1-4C-alkyl, 3-7C-cycloalkyl or phenyl,
R83 is hydrogen or 1-4C-alkyl, or
R82 and R83 together and with inclusion of the nitrogen atom, to which they are bound, form a heterocyclic ring radical selected from the group consisting of pyrrolidinyl and piperidinyl,
R9 is 1-4C-alkyl,
   and the salts, stereoisomers, hydrates and hydrates of the salts of these compounds.

Compounds according to aspect c in particular worthy to be mentioned are those of formula I, in which
R1 is chlorine, fluorine, nitro, amino, methyl, methoxy, methoxyethoxy or difluoromethoxy,
R2 is methoxy,
R3 is methoxy,
   and none of R1, R2 and R3 is bound to the 10-position of the pyrrolo[2.1-a]isoquinoline ring,
R4 is hydrogen or methyl,
R41 is hydrogen or methyl,
R5 is hydrogen,
R51 is hydrogen,
   or
R4 is hydrogen,
R41 is hydrogen,
R5 is hydrogen,
R51 is hydrogen,
R6 is methyl, ethyl or methoxycarbonylethyl,
R7 is phenyl, Het2, R71- and/or R72- and/or R73-substituted phenyl, or naphthyl, in which
Het2 is indolyl, pyridinyl or quinolyl,
R71 is hydroxyl, chlorine, methoxy, dimethylamino, or aryloxy, in which
aryl is R711-substituted phenyl, in which
R711 is chlorine,
R72 is methyl, tert-butyl or methoxy,
R73 is methyl, tert-butyl or methoxy,
R8 is phenyl, phenylcarbonyl, —C(O)—N(R82)R83 or —C(O)—OR9, in which
R82 is hydrogen, methyl, ethyl, iso-propyl, iso-butyl, cyclohexyl, cyclopropyl or phenyl,
R83 is hydrogen or methyl, or
R82 and R83 together and with inclusion of the nitrogen atom, to which they are bound, form a pyrrolidinyl radical,
R9 is methyl or ethyl,
   and the salts, stereoisomers, hydrates and hydrates of the salts of these compounds.

Compounds according to aspect d more worthy to be mentioned are those of formula I, in which
R1 is halogen, nitro, amino, 1-4C-alkyl, 1-4C-alkoxy, 1-4C-alkoxy-2-4C-alkoxy, or completely or predominantly fluorine-substituted 1-4C-alkoxy,
R2 is hydrogen or 1-4C-alkoxy,
R3 is hydrogen or 1-4C-alkoxy, or
R1 and R2 bound to the benzo ring moiety in ortho-position to each other together form a completely or predominantly fluorine-substituted 1-2C-alkylenedioxy bridge and R3 is hydrogen,
R4 is 1-4C-alkyl,
R41 is hydrogen or 1-4C-alkyl,
R5 is hydrogen,
R51 is hydrogen,
R6 is 1-6C-alkyl or 1-4C-alkyl substituted by R61, in which
R61 is 1-4C-alkoxycarbonyl,
R7 is phenyl, Het2, R71- and/or R72- and/or R73-substituted phenyl, R74- and/or R75-substituted Het2, or naphthyl, in which
Het2 is a heteroaryl radical selected from the group consisting of furanyl, thiophenyl, pyrrolyl, pyridinyl, quinolyl, indolyl, benzothiophenyl and benzofuranyl,
R71 is hydroxyl, halogen, nitro, trifluoromethyl, 1-4C-alkyl, 1-4C-alkoxy, amino, mono- or di-1-4C-alkylamino, 1-4C-alkylsulphonylamino, tolylsulphonylamino or aryloxy, in which
aryl is R711-substituted phenyl, in which
R711 is halogen,
R72 is 1-4C-alkyl, 1-4C-alkoxy or 1-4C-alkoxycarbonyl,
R73 is 1-4C-alkyl or 1-4C-alkoxy,
R74 is 1-4C-alkyl, trifluoromethyl, 1-4C-alkoxy, 1-4C-alkoxycarbonyl, nitro, phenyl or phenyloxy,
R75 is 1-4C-alkyl,
R8 is phenyl, phenylcarbonyl, —C(O)—N(R82)R83 or —C(O)—OR9, in which
R82 is hydrogen, 1-4C-alkyl, 3-7C-cycloalkyl or phenyl,
R83 is hydrogen or 1-4C-alkyl, or
R82 and R83 together and with inclusion of the nitrogen atom, to which they are bound, form a heterocyclic ring radical selected from the group consisting of pyrrolidinyl and piperidinyl,
R9 is 1-4C-alkyl,
   and the salts, stereoisomers, hydrates and hydrates of the salts of these compounds.

Compounds according to aspect d further more worthy to be mentioned are those of formula I, in

which

R1 is halogen, nitro, amino, 1-4C-alkyl, 1-4C-alkoxy, 1-4C-alkoxy-2-4C-alkoxy, or completely or predominantly fluorine-substituted 1-4C-alkoxy,
R2 is 1-4C-alkoxy,
R3 is hydrogen,
   and none of R1, R2 and R3 is bound to the 10-position of the pyrrolo[2.1-a]isoquinoline ring, in particular none of R1 and R2 is bound to the 7- or 10-position of the pyrrolo[2.1-a]isoquinoline ring,
R4 is 1-4C-alkyl,
R41 is hydrogen or 1-4C-alkyl,
R5 is hydrogen,
R51 is hydrogen,
R6 is 1-6C-alkyl, formyl, or 1-4C-alkyl substituted by R61, in which
R61 is 1-4C-alkoxycarbonyl,
R7 is phenyl, Het2, R71- and/or R72- and/or R73-substituted phenyl, R74- and/or R75-substituted Het2, or naphthyl, in which
Het2 is a heteroaryl radical selected from the group consisting of furanyl, thiophenyl, pyrrolyl, pyridinyl, quinolyl, indolyl, benzothiophenyl and benzofuranyl,
R71 is hydroxyl, 1-4C-alkoxy, amino or mono- or di-1-4C-alkylamino,
R72 is 1-4C-alkyl or 1-4C-alkoxy,
R73 is 1-4C-alkyl or 114 alkoxy,
R74 is 1-4C-alkyl, trifluoromethyl, 1-4C-alkoxy, 1-4C-alkoxycarbonyl, nitro, phenyl or phenyloxy,
R75 is 1-4C-alkyl,
R8 is phenyl, phenylcarbonyl, —C(O)—N(R82)R83 or, in particular, —C(O)—OR9, in which
R82 is hydrogen, 1-4C-alkyl, 3-7C-cycloalkyl or phenyl,
R83 is hydrogen or 1-4C-alkyl, or
R82 and R83 together and with inclusion of the nitrogen atom, to which they are bound, form a heterocyclic ring radical selected from the group consisting of pyrrolidinyl and piperidinyl,
R9 is 1-4C-alkyl,
   and the salts, stereoisomers, hydrates and hydrates of the salts of these compounds.

Compounds according to aspect d in particular worthy to be mentioned are those of formula I, in which
R1 is chlorine, fluorine, nitro, amino, methyl, methoxy, methoxyethoxy or difluoromethoxy,
R2 is hydrogen or methoxy,
R3 is hydrogen or methoxy, or
R1 and R2 bound to the benzo ring moiety in ortho-position to each other together form a difluoromethylenedioxy bridge and R3 is hydrogen,
   and none of R1, R2 and R3 is bound to the 10-position of the pyrrolo[2.1-a]isoquinoline ring,
R4 is methyl,
R41 is hydrogen or methyl,
R5 is hydrogen,
R51 is hydrogen,
   or
R4 is hydrogen,
R41 is hydrogen,
R5 is methyl,
R51 is hydrogen,
R6 is methyl, ethyl or methoxycarbonylethyl,
R7 is phenyl, Het2, R71- and/or R72- and/or R73-substituted phenyl, or naphthyl, in which
Het2 is a heteroaryl radical selected from the group consisting of furanyl, thiophenyl, pyrrolyl, pyridinyl, quinolyl, indolyl, benzothiophenyl and benzofuranyl,
R71 is hydroxyl, chlorine, methoxy, dimethylamino, or aryloxy, in which
aryl is R711-substituted phenyl, in which
R711 is chlorine,
R72 is methyl, tert-butyl or methoxy,
R73 is methyl, tert-butyl or methoxy,
R8 is phenyl, phenylcarbonyl, —C(O)—N(R82)R83 or —C(O)—OR9, in which
R82 is hydrogen, methyl, ethyl, iso-propyl, iso-butyl, cyclohexyl, cyclopropyl or phenyl,
R83 is hydrogen or methyl, or
R82 and R83 together and with inclusion of the nitrogen atom, to which they are bound, form a pyrrolidinyl radical,
R9 is methyl or ethyl,
   and the salts, stereoisomers, hydrates and hydrates of the salts of these compounds.

Compounds according to aspect d in further particular worthy to be mentioned are those of formula I, in which

either

R1 is bonded in the 8-position of the pyrrolodihydroisoquinoline scaffold, and is chlorine, methoxy, methoxyethoxy or difluoromethoxy,
R2 is bonded in the 9-position of the pyrrolodihydroisoquinoline scaffold, and is methoxy,
R3 is hydrogen,
   or
R1 is bonded in the 9-position of the pyrrolodihydroisoquinoline scaffold, and is chlorine, fluorine, methoxy, nitro, methyl, amino, or difluoromethoxy,
R2 is bonded in the 8-position of the pyrrolodihydroisoquinoline scaffold, and is methoxy,
R3 is hydrogen,
R4 is methyl,
R41 is hydrogen or methyl,
R5 is hydrogen,
R51 is hydrogen,
R6 is methyl, ethyl or methoxycarbonylethyl,
R7 is phenyl, Het2, R71- and/or R72- and/or R73-substituted phenyl, or naphthyl, in which
Het2 is indolyl, pyridinyl or quinolyl,
R71 is hydroxyl, methoxy or dimethylamino, in which
R72 is methyl, tert-butyl or methoxy,
R73 is methyl, tert-butyl or methoxy,
R8 is phenylcarbonyl, —C(O)—N(R82)R83 or, in particular, —C(O)—OR9, in which
R82 is hydrogen, methyl, ethyl, iso-propyl, iso-butyl, cyclohexyl, cyclopropyl or phenyl,
R83 is hydrogen or methyl, or
R82 and R83 together and with inclusion of the nitrogen atom, to which they are bound, form a pyrrolidinyl radical,
R9 is methyl or ethyl,
   and the salts, stereoisomers, hydrates and hydrates of the salts of these compounds.

A special interest of the compounds according to this invention refers to those compounds of formula I which are included, within the scope of this invention, by one or, when possible, by more of the following embodiments:

A special embodiment (embodiment a) of the compounds according to this invention refers to those compounds of formula I, in which
R8 is —C(O)—OR9.

Another special embodiment (embodiment b) of the compounds according to this invention refers to those compounds of formula I, in which
R8 is phenylcarbonyl.

Another special embodiment (embodiment c) of the compounds according to this invention refers to those compounds of formula I, in which
R8 is phenyl.

Another special embodiment (embodiment d) of the compounds according to this invention refers to those compounds of formula I, in which
R8 is —C(O)—N(R82)R83.

Another special embodiment (embodiment e) of the compounds according to this invention refers to those compounds of formula I, in which
R4, R41, R5 and R51 are all hydrogen.

Another special embodiment (embodiment f) of the compounds according to this invention refers to those compounds of formula I, in which
R4 is 1-4C-alkyl, such as e.g. methyl,
R41 is hydrogen,
R5 is hydrogen,
R51 is hydrogen.

Another special embodiment (embodiment g) of the compounds according to this invention refers to those compounds of formula I, in which
R4 is 1-4C-alkyl, such as e.g. methyl,
R41 is 1-4C-alkyl, such as e.g. methyl,
R5 is hydrogen,
R51 is hydrogen.

Another special embodiment (embodiment h) of the compounds according to this invention refers to those compounds of formula I, in which

none of R1, R2 and R3 is bound to the 10-position of the pyrrolo[2.1-a]isoquinoline ring.

Another special embodiment (embodiment i) of the compounds according to this invention refers to those compounds of formula I, in which

none of R1 and R2 is bound to the 7- or 10-position of the pyrrolo[2.1-a]isoquinoline ring, and

R3 is hydrogen.

Another special embodiment (embodiment J) of the compounds according to this invention refers to those compounds of formula I, in which
R1 is 1-4C-alkoxy, hydroxyl, 1-4C-alkoxy-2-4C-alkoxy, 3-7C-cycloalkoxy, 3-7C-cycloalkylmethoxy, or
   completely or predominantly fluorine-substituted 1-4C-alkoxy,
R2 is halogen or 1-4C-alkoxy,
R3 is hydrogen.

Another special embodiment (embodiment k) of the compounds according to this invention refers to those compounds of formula I, in which
R1 is 1-4C-alkoxy, hydroxyl, 1-4C-alkoxy-2-4C-alkoxy, 3-7C-cycloalkoxy, 3-7C-cycloalkylmethoxy, or
   completely or predominantly fluorine-substituted 1-4C-alkoxy,
R2 is halogen or 1-4C-alkoxy,
R3 is 1-4C-alkoxy.

Another special embodiment (embodiment l) of the compounds according to this invention refers to those compounds of formula I, in which
R1 is 1-4C-alkoxy, 1-4C-alkoxy-2-4C-alkoxy, or completely or predominantly fluorine-substituted 1-4C-alkoxy,
R2 is 1-4C-alkoxy,
R3 is hydrogen;
   in particular
R1 is 1-2C-alkoxy, 1-2C-alkoxy-ethoxy, or completely or predominantly fluorine-substituted 1-2C-alkoxy,
R2 is 1-2C-alkoxy,
R3 is hydrogen.

Another special embodiment (embodiment m) of the compounds according to this invention refers to those compounds of formula I, in which
R1 is 1-4C-alkoxy, 1-4C-alkoxy-2-4C-alkoxy, or completely or predominantly fluorine-substituted 1-4C-alkoxy,
R2 is halogen,
R3 is hydrogen;
   in particular
R1 is 1-2C-alkoxy, 1-2C-alkoxy-ethoxy, or completely or predominantly fluorine-substituted 1-2C-alkoxy,
R2 is chlorine or fluorine,
R3 is hydrogen;
   in more particular
R1 is 1-2C-alkoxy,
R2 is chlorine or fluorine,
R3 is hydrogen.

Another special embodiment (embodiment n) of the compounds according to this invention refers to those compounds of formula I, in which
R1 is halogen, nitro, amino, 1-4C-alkyl, 1-4C-alkoxy, 1-4C-alkoxy-2-4C-alkoxy, or completely or predominantly fluorine-substituted 1-4C-alkoxy,
R2 is halogen or 1-4C-alkoxy,
R3 is hydrogen.

Another special embodiment (embodiment o) of the compounds according to this invention refers to those compounds of formula I, in which
R1 is halogen, 1-4C-alkyl, 1-4C-alkoxy, 1-4C-alkoxy-2-4C-alkoxy, or completely or predominantly fluorine-substituted 1-4C-alkoxy,
R2 is 1-4C-alkoxy,
R3 is 1-4C-alkoxy.

Another special embodiment (embodiment p) of the compounds according to this invention refers to those compounds of formula I, in which
R1 is bonded in the 8-position of the pyrrolo[2.1-a]isoquinoline ring, and is halogen, 1-4C-alkoxy-2-4C-alkoxy, or completely or predominantly fluorine-substituted 1-4C-alkoxy,
R2 is bonded in the 9-position of the pyrrolo[2.1-a]isoquinoline ring, and is 1-4C-alkoxy,
R3 is hydrogen.

Another special embodiment (embodiment q) of the compounds according to this invention refers to those compounds of formula I, in which
R1 is bonded in the 9-position of the pyrrolo[2.1-a]isoquinoline ring, and is halogen, nitro, methyl, amino, or completely or predominantly fluorine-substituted 1-4C-alkoxy,
R2 is bonded in the 8-position of the pyrrolo[2.1-a]isoquinoline ring, and is 1-4C-alkoxy,
R3 is hydrogen.

Each and every group of compounds of formula I according to this invention, in which R1, R2 and R3 bonded to the left benzo ring of the dihydroisoquinoline moiety of the pyrrolo[2.1-a]isoquinoline ring constitute any one of the substitution patterns as shown and specified in the following represents a respective further independent special embodiment of the compounds according to this invention: 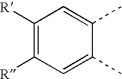
in which
either, in a first independent special embodiment,
R′ is 1-2C-alkoxy, such as e.g. methoxy; and R″ is 1-2C-alkoxy, such as e.g. methoxy;
or, in a second independent special embodiment,
R′ is 1-2C-alkoxy, such as e.g. methoxy; and R″ is difluoromethoxy;
or, in a third independent special embodiment,
R′ is chlorine; and R″ is 1-2C-alkoxy, such as e.g. methoxy;
or, in a fourth independent special embodiment,
R′ is 1-2C-alkoxy, such as e.g. methoxy; and R″ is chlorine;
or, in a fifth independent special embodiment,
R′ is 2-methoxy-ethoxy; and R″ is 1-2C-alkoxy, such as e.g. methoxy;
or, in a sixth independent special embodiment,
R′ is difluoromethoxy; and R″ is 1-2C-alkoxy, such as e.g. methoxy;
or, in a seventh independent special embodiment,
R′ is 1-2C-alkoxy, such as e.g. methoxy; and R″ is fluorine.

Another special embodiment (embodiment r) of the compounds according to this invention refers to those compounds of formula I, in which
R7 is naphthyl (such as e.g. naphthalen-1-yl), 4-hydroxy-3,5-dimethylphenyl, 3-dimethylamino-phenyl, 3,4,5-trimethoxy-phenyl, pyridinyl or quinolin-4-yl.

Another special embodiment (embodiment s) of the compounds according to this invention refers to those compounds of formula I, in which
R7 is 4-hydroxy-3,5-dimethylphenyl.

Another special embodiment (embodiment t) of the compounds according to this invention refers to those compounds of formula I, in which
R7 is 3-dimethylamino-phenyl.

Another special embodiment (embodiment u) of the compounds according to this invention refers to those compounds of formula I, in which
R7 is Het2, in which
Het2 is a fused bicyclic 9- or 10-membered heteroaryl radical comprising one to three heteroatoms, each of which is selected from a group consisting of nitrogen, oxygen and sulfur, which optionally contains a benzene ring,
   such as e.g. quinolyl or indolyl.

Another special embodiment (embodiment v) of the compounds according to this invention refers to those compounds of formula I, in which
R6 is 1-4C-alkyl, such as e.g. methyl.

Another special embodiment (embodiment w) of the compounds according to this invention refers to those compounds of formula I, in which
R6 is 1-4C-alkyl substituted by 1-4C-alkoxycarbonyl, such as e.g. 2-methoxycarbonyl-ethyl.

Another special embodiment (embodiment x) of the compounds according to this invention refers to those compounds of formula I, in which
R1 is halogen, amino, 1-4C-alkyl, 1-4C-alkoxy, hydroxyl, 1-4C-alkoxy-2-4C-alkoxy, 3-7C-cycloalkoxy, 3-7C-cycloalkylmethoxy, or completely or predominantly fluorine-substituted 1-4C-alkoxy,
R2 is 1-4C-alkoxy,
R3 is 1-4C-alkoxy,
   and
R4 is 1-4C-alkyl, such as e.g. methyl,
R41 is hydrogen, or 1-4C-alkyl, such as e.g. methyl,
R5 is hydrogen,
R51 is hydrogen.

A notable embodiment of the compounds according to this invention refers to those compounds of formula I, in which
R1 is 1-4C-alkoxy,
R2 is hydrogen or 1-4C-alkoxy,
R3 is hydrogen, or
R1 and R2 bound to the benzo ring moiety in ortho-position to each other together form an 1-2C-alkylenedioxy bridge and R3 is hydrogen,
   and none of R1, R2 and R3 is bound to the 10-position of the pyrrolo[2.1-a]isoquinoline ring,
R4 is 1-4C-alkyl,
R41 is hydrogen or 1-4C-alkyl,
R5 is hydrogen,
R51 is hydrogen,
R6 is 1-6C-alkyl, or R61-substituted 1-4C-alkyl, particularly R6 is methyl, in which
R61 is 1-4C-alkoxycarbonyl,
R8 is —C(O)—OR9, in which
R9 is 1-4C-alkyl, particularly ethyl.

Yet a notable embodiment of the compounds according to this invention refers to those compounds of formula I, in which
R1 is halogen, 1-4C-alkyl, 1-4C-alkoxy, 1-4C-alkoxy-2-4C-alkoxy, or completely or predominantly fluorine-substituted 1-4C-alkoxy,
R2 is 1-4C-alkoxy,
R3 is 1-4C-alkoxy, and none of R1, R2 and R3 is bound to the 10-position of the pyrrolo[2.1-a]isoquinoline ring,
R4 is hydrogen,
R41 is hydrogen,
R5 is hydrogen,
R51 is hydrogen,
R6 is 1-6C-alkyl, or R61-substituted 1-4C-alkyl, particularly R6 is methyl, in which
R61 is 1-4C-alkoxycarbonyl,
R8 is —C(O)—OR9, in which
R9 is 1-4C-alkyl, particularly ethyl.

Still a notable embodiment of the compounds according to this invention refers to those compounds of formula I, in which
R1 is 1-4C-alkoxy-2-4C-alkoxy, 3-7C-cycloalkoxy, 3-7C-cycloalkylmethoxy, or completely or predominantly fluorine-substituted 1-4C-alkoxy,
R2 is halogen,
R3 is 1-4C-alkoxy,
   and none of R1, R2 and R3 is bound to the 10-position of the pyrrolo[2.1-a]isoquinoline ring,
R4 is hydrogen,
R41 is hydrogen,
R5 is hydrogen,
R51 is hydrogen,
R6 is 1-6C-alkyl, or R61-substituted 1-4C-alkyl, particularly R6 is methyl, in which
R61 is 1-4C-alkoxycarbonyl,
R8 is —C(O)—OR9, in which
R9 is 1-4C-alkyl, particularly ethyl.

Still yet a notable embodiment of the compounds according to this invention refers to those compounds of formula I, in which R1 is halogen,
R2 is 1-4C-alkoxy,
R3 is 1-4C-alkoxy,
   and none of R1, R2 and R3 is bound to the 10-position of the pyrrolo[2.1-a]isoquinoline ring,
R4 is hydrogen,
R41 is hydrogen,
R5 is hydrogen,
R51 is hydrogen,
R6 is 1-6C-alkyl, or R61-substituted 1-4C-alkyl, particularly R6 is methyl, in which
R61 is 1-4C-alkoxycarbonyl,
R8 is —C(O)—OR9, in which
R9 is 1-4C-alkyl, particularly ethyl.

A further notable embodiment of the compounds according to this invention refers to those compounds of formula I, in which
R1 is halogen, 1-4C-alkyl, 1-4C-alkoxy, 1-4C-alkoxy-2-4C-alkoxy, or completely or predominantly fluorine-substituted 1-4C-alkoxy,
R2 is 1-4C-alkoxy,
R3 is 1-4C-alkoxy,
   and none of R1, R2 and R3 is bound to the 10-position of the pyrrolo[2.1-a]isoquinoline ring,
R4 is hydrogen,
R41 is hydrogen,
R5 is hydrogen,
R51 is hydrogen,
R6 is 1-4C-alkyl, or R61-substituted 1-4C-alkyl, particularly R6 is methyl, in which
R61 is 1-4C-alkoxycarbonyl,
R7 is Het2, in which
Het2 optionally contains a benzene ring, and is a fused bicyclic 9- or 10-membered heteroaryl radical comprising one to three heteroatoms, each of which is selected from a group consisting of nitrogen, oxygen and sulfur,
R8 is —C(O)—OR9, in which
R9 is 1-4C-alkyl, particularly ethyl.

Yet a further notable embodiment of the compounds according to this invention refers to those compounds of formula I, in which
R1 is 1-4C-alkyl, 1-4C-alkoxy-2-4C-alkoxy, or completely or predominantly fluorine-substituted 1-4C-alkoxy,
R2 is halogen,
R3 is 1-4C-alkoxy,
   and none of R1, R2 and R3 is bound to the 10-position of the pyrrolo[2.1-a]isoquinoline ring,
R4 is hydrogen,
R41 is hydrogen,
R5 is hydrogen,
R51 is hydrogen,
R6 is 1-4C-alkyl, or R61-substituted 1-4C-alkyl, particularly R6 is methyl, in which
R61 is 1-4C-alkoxycarbonyl,
R7 is Het2, in which
Het2 optionally contains a benzene ring, and is a fused bicyclic 9 or 10-membered heteroaryl radical comprising one to three heteroatoms, each of which is selected from a group consisting of nitrogen, oxygen and sulfur,
R8 is —C(O)—OR9, in which
R9 is 1-4C-alkyl, particularly ethyl.

Another notable embodiment of the compounds according to this invention refers to those compounds of formula I, in which
R1 is halogen, 1-4C-alkyl, 1-4C-alkoxy, 1-4C-alkoxy-2-4C-alkoxy, or completely or predominantly fluorine-substituted 1-4C-alkoxy,
R2 is 1-4C-alkoxy,
R3 is 1-4C-alkoxy,
   and none of R1, R2 and R3 is bound to the 10-position of the pyrrolo[2,1-a]isoquinoline ring,
R4 is 1-4C-alkyl,
R41 is hydrogen or 1-4C-alkyl,
R5 is hydrogen,
R51 is hydrogen,
R6 is 1-6C-alkyl, or R61-substituted 1-4C-alkyl, particularly R6 is methyl, in which
R61 is 1-4C-alkoxycarbonyl,
R8 is —C(O)—OR9, in which
R9 is 1-4C-alkyl, particularly ethyl.

Yet another notable embodiment of the compounds according to this invention refers to those compounds of formula I, in which
R1 is 1-4C-alkoxy-2-4C-alkoxy, 3-7C-cycloalkoxy, 3-7C-cycloalkylmethoxy, or completely or predominantly fluorine-substituted 1-4C-alkoxy,
R2 is halogen,
R3 is 1-4C-alkoxy,
   and none of R1, R2 and R3 is bound to the 10-position of the pyrrolo[2.1-a]isoquinoline ring,
R4 is 1-4C-alkyl,
R41 is hydrogen or 1-4C-alkyl,
R5 is hydrogen,
R51 is hydrogen,
R6 is 1-6C-alkyl, or R61-substituted 1-4C-alkyl, particularly R6 is methyl, in which
R61 is 1-4C-alkoxycarbonyl,
R8 is —C(O)—OR9, in which
R9 is 1-4C-alkyl, particularly ethyl.

Still yet another notable embodiment of the compounds according to this invention refers to those compounds of formula I, in which
R1 is halogen,
R2 is 1-4C-alkoxy,
R3 is 1-4C-alkoxy,
   and none of R1, R2 and R3 is bound to the 10-position of the pyrrolo[2.1-a]isoquinoline ring,
R4 is 1-4C-alkyl,
R41 is hydrogen or 1-4C-alkyl,
R5 is hydrogen,
R51 is hydrogen,
R6 is 1-6C-alkyl, or R61-substituted 1-4C-alkyl, particularly R6 is methyl, in which
R61 is 1-4C-alkoxycarbonyl,
R8 is —C(O)—OR9, in which
R9 is 1-4C-alkyl, particularly ethyl.

Another further notable embodiment of the compounds according to this invention refers to those compounds of formula I, in which
R1 is halogen, 1-4C-alkyl, 1-4C-alkoxy, 1-4C-alkoxy-2-4C-alkoxy, or completely or predominantly fluorine-substituted 1-4C-alkoxy, —
R2 is 1-4C-alkoxy,
R3 is 1-4C-alkoxy,
   and none of R1, R2 and R3 is bound to the 10-position of the pyrrolo[2.1-a]isoquinoline ring,
R4 is 1-4C-alkyl,
R41 is hydrogen or 1-4C-alkyl,
R5 is hydrogen,
R51 is hydrogen,
R6 is 1-4C-alkyl, or R61-substituted 1-4C-alkyl, particularly R6 is methyl, in which
R61 is 1-4C-alkoxycarbonyl,
R7 is Het2, in which
Het2 optionally contains a benzene ring, and is a fused bicyclic 9- or 10-membered heteroaryl radical comprising one to three heteroatoms, each of which is selected from a group consisting of nitrogen, oxygen and sulfur,
R8 is —C(O)—OR9, in which
R9 is 1-4C-alkyl, particularly ethyl.

Yet another further notable embodiment of the compounds according to this invention refers to those compounds of formula I, in which
R1 is 1-4C-alkyl, 1-4C-alkoxy-2-4C-alkoxy, or completely or predominantly fluorine-substituted 1-4C-alkoxy,
R2 is halogen,
R3 is 1-4C-alkoxy,
   and none of R1, R2 and R3 is bound to the 10-position of the pyrrolo[2.1-a]isoquinoline ring,
R4 is 1-4C-alkyl,
R41 is hydrogen or 1-4C-alkyl,
R5 is hydrogen,
R51 is hydrogen,
R6 is 1-6C-alkyl, or R61-substituted 1-4C-alkyl, particularly R6 is methyl, in which
R61 is 1-4C-alkoxycarbonyl,
R7 is Het2, in which
Het2 optionally contains a benzene ring, and is a fused bicyclic 9 or 10-membered heteroaryl radical comprising one to three heteroatoms, each of which is selected from a group consisting of nitrogen, oxygen and sulfur,
R8 is —C(O)—OR9, in which
R9 is 1-4C-alkyl, particularly ethyl.

In a facet of this invention (facet 1), compounds according to this invention more worthy to be mentioned are those compounds of formula I,

in which, in a first embodiment,

either

R1 is halogen, 1-4C-alkyl, nitro, amino, 1-4C-alkoxy-2-4C-alkoxy, or completely or predominantly fluorine-substituted 1-4C-alkoxy, and
R2 is 1-4C-alkoxy,
   or
R1 is 1-4C-alkoxy, 1-4C-alkyl, nitro, amino, 1-4C-alkoxy-2-4C-alkoxy, or completely or predominantly fluorine-substituted 1-4C-alkoxy, and
R2 is halogen,
R3 is hydrogen,
   either
R4 is hydrogen,
R41 is hydrogen,
R5 is hydrogen, and
R51 is hydrogen,
   or
R4 is 1-4C-alkyl,
R41 is hydrogen or 1-4C-alkyl,
R5 is hydrogen, and
R51 is hydrogen;
   or in which, in a second embodiment,
R1 is 1-4C-alkoxy,
R2 is 1-4C-alkoxy,
R3 is hydrogen,
R4 is hydrogen,
R41 is hydrogen,
R5 is hydrogen,
R51 is hydrogen;
R6 is 1-4C-alkyl, or 1-4C-alkyl substituted by R61, in which
R61 is 1-4C-alkoxycarbonyl,
R7 is Het2, R71- and/or R72- and/or R73-substituted phenyl, or naphthyl, in which
Het2 is a monocyclic or fused bicyclic 5- to 10-membered heteroaryl radical comprising one to three heteroatoms, each of which is selected from a group consisting of nitrogen, oxygen and sulfur,
R71 is hydroxyl, 1-4C-alkoxy or di-1-4C-alkylamino,
R72 is 1-4C-alkyl or 1-4C-alkoxy,
R73 is 1-4C-alkyl or 1-4C-alkoxy,
R8 is phenyl, phenylcarbonyl, —C(O)—N(R82)R83, or, in particular, —C(O)—OR9, in which
R82 is hydrogen, 1-4C-alkyl, 3-7C-cycloalkyl, or phenyl,
R83 is hydrogen, 1-4C-alkyl,
or R82 and R83 together and with inclusion of the nitrogen atom, to which they are bonded form a pyrrolidinyl or piperidinyl ring,
R9 is 1-4C-alkyl,
   and the salts, stereoisomers, hydrates and hydrates of the salts of these compounds.

Also in the meaning of facet 1 of this invention, compounds according to this invention in particular worthy to be mentioned are those compounds of formula I, in which

either

R1 is chlorine, fluorine, 1-2C-alkyl, nitro, amino, 1-2C-alkoxy-ethoxy, or completely or predominantly fluorine-substituted 1-2C-alkoxy, and
R2 is 1-2C-alkoxy,
   or
R1 is 1-2C-alkoxy, 1-2C-alkyl, nitro, amino, 1-2C-alkoxy-ethoxy, or completely or predominantly fluorine-substituted 1-2C-alkoxy, and
R2 is halogen,
R3 is hydrogen,
   and none of R1 and R2 is bound to the 7- or 10-position of the pyrrolo[2.1-a]isoquinoline ring,
   either
R4 is hydrogen,
R41 is hydrogen,
R5 is hydrogen, and
R51 is hydrogen,
   or
R4 is i-2C-alkyl,
R41 is hydrogen or 1-2C-alkyl,
R5 is hydrogen, and
R51 is hydrogen,
R6 is 1-2C-alkyl, or 1-2C-alkyl substituted by R61, in which
R61 is 1-2C-alkoxycarbonyl,
R7 is naphthyl (such as e.g. naphthalen-1-yl), 4-hydroxy-3,5-dimethylphenyl, 3-dimethylamino-phenyl or quinolinyl,
R8 is —C(O)—OR9, in which
R9 is 1-2C-alkyl,
   and the salts, stereoisomers, hydrates and hydrates of the salts of these compounds.

Also in the meaning of facet 1 of this invention, yet compounds according to this invention in particular worthy to be mentioned are those compounds of formula I, in which
R1 is 1-2C-alkoxy,
R2 is 1-2C-alkoxy,
R3 is hydrogen,
   and none of R1 and R2 is bound to the 7- or 10-position of the pyrrolo[2.1-a]isoquinoline ring,
R4 is 1-2C-alkyl,
R41 is hydrogen or 1-2C-alkyl,
R5 is hydrogen,
R51 is hydrogen,
R6 is 1-2C-alkyl, or 1-2C-alkyl substituted by R61, in which
R61 is 1-2C-alkoxycarbonyl,
R7 is naphthyl (such as e.g. naphthalen-1-yl), 4-hydroxy-3,5-dimethylphenyl, 3-dimethylamino-phenyl or quinolinyl,
R8 is —C(O)—OR9, in which
R9 is 1-2C-alkyl,
   and the salts, stereoisomers, hydrates and hydrates of the salts of these compounds.

Also in the meaning of facet 1 of this invention, compounds according to this invention in more particular worthy to be mentioned are those compounds of formula I, in which

either

R1 is bonded in the 8-position of the pyrrolo[2.1-a]isoquinoline ring, and is chlorine, 2-methoxy-ethoxy or difluoromethoxy, and R2 is bonded in the 9-position of the pyrrolo[2.1-a]isoquinoline ring, and is 1-2C-alkoxy,
   or
R1 is bonded in the 9-position of the pyrrolo[2.1-a]isoquinoline ring, and is chlorine, fluorine, nitro, methyl, amino or difluoromethoxy, and
R2 is bonded in the 8-position of the pyrrolo[2.1-a]isoquinoline ring, and is 1-2C-alkoxy,
R3 is hydrogen;
R4 is hydrogen,
R41 is hydrogen,
R5 is hydrogen,
R51 is hydrogen,
R6 is methyl or 2-methoxycarbonylethyl;
R7 is naphthyl (such as e.g. naphthalen-1-yl), 4-hydroxy-3,5-dimethylphenyl, 3-dimethylamino-phenyl or quinolin-4-yl;
R8 is —C(O)—OR9, in which
R9 is 1-2C-alkyl;
   and the salts, stereoisomers, hydrates and hydrates of the salts of these compounds.

Also in the meaning of facet 1 of this invention, yet compounds according to this invention in more particular worthy to be mentioned are those compounds of formula I, in which
R1 is bonded in the 8-position of the pyrrolo[2.1-a]isoquinoline ring, and is methoxy,
R2 is bonded in the 9-position of the pyrrolo[2.1-a]isoquinoline ring, and is methoxy,
R3 is hydrogen;
R4 is methyl,
R41 is methyl or hydrogen,
R5 is hydrogen,
R51 is hydrogen,
R6 is methyl or 2-methoxycarbonylethyl;
R7 is naphthyl (such as e.g. naphthalen-1-yl), 4-hydroxy-3,5-dimethylphenyl, 3-dimethylamino-phenyl or quinolinyl;
R8 is —C(O)—OR9, in which
R9 is 1-2C-alkyl;
   and the salts, stereoisomers, hydrates and hydrates of the salts of these compounds.

Also in the meaning of facet 1 of this invention, compounds according to this invention in further more particular worthy to be mentioned are those compounds of formula I, in which

either

R1 is bonded in the 8-position of the pyrrolo[2.1-a]isoquinoline ring, and is chlorine, 2-methoxy-ethoxy or difluoromethoxy, and
R2 is bonded in the 9-position of the pyrrolo[2.1-a]isoquinoline ring, and is 1-2C-alkoxy,
   or
R1 is bonded in the 9-position of the pyrrolo[2.1-a]isoquinoline ring, and is chlorine, fluorine or difluoromethoxy, and
R2 is bonded in the 8-position of the pyrrolo[2.1-a]isoquinoline ring, and is 1-2C-alkoxy,
R3 is hydrogen;
   either
R4 is hydrogen,
R41 is hydrogen,
R5 is hydrogen, and
R51 is hydrogen,
   or
R4 is methyl,
R41 is hydrogen or methyl,
R5 is hydrogen, and
R51 is hydrogen;
R6 is methyl;
R7 is 4-hydroxy-3,5-dimethylphenyl or 3-dimethylamino-phenyl;
R8 is —C(O)—OR9, in which
R9 is ethyl;
   and the salts, stereoisomers, hydrates and hydrates of the salts of these compounds.

Also in the meaning of facet 1 of this invention, yet compounds according to this invention in further more particular worthy to be mentioned are those compounds of formula I, in which
R1 is bonded in the 8-position of the pyrrolo[2.1-a]isoquinoline ring, and is methoxy,
R2 is bonded in the 9-position of the pyrrolo[2.1-a]isoquinoline ring, and is methoxy,
R3 is hydrogen;
R4 is methyl,
R41 is hydrogen or methyl,
R5 is hydrogen,
R51 is hydrogen;
R6 is methyl;
R7 is 4-hydroxy-3,5-dimethylphenyl or 3-dimethylamino-phenyl;
R8 is —C(O)—OR9, in which
R9 is ethyl;
   and the salts, stereoisomers, hydrates and hydrates of the salts of these compounds.

Also in the meaning of facet 1 of this invention, compounds according to this invention in still further more particular worthy to be mentioned are those compounds of formula I, in which

either

R1 is bonded in the 8-position of the pyrrolo[2.1-a]isoquinoline ring, and is chlorine, 2-methoxy-ethoxy or difluoromethoxy, and
R2 is bonded in the 9-position of the pyrrolo[2.1-a]isoquinoline ring, and is methoxy,
   or
R1 is bonded in the 9-position of the pyrrolo[2.1-a]isoquinoline ring, and is chlorine, fluorine or difluoromethoxy, and
R2 is bonded in the 8-position of the pyrrolo[2.1-a]isoquinoline ring, and is methoxy,
R3 is hydrogen;
   either
R4 is hydrogen,
R41 is hydrogen,
R5 is hydrogen, and
R51 is hydrogen,
   or
R4 is methyl,
R41 is hydrogen or methyl,
R5 is hydrogen, and
R51 is hydrogen;
R6 is methyl;
R7 is naphthalen-1-yl, 4-hydroxy-3,5-dimethylphenyl, 3-dimethylamino-phenyl or quinolin-4-yl;
R8 is —C(O)—OR9, in which
R9 is ethyl;
   and the salts, stereoisomers, hydrates and hydrates of the salts of these compounds.

Also in the meaning of facet 1 of this invention, yet compounds according to this invention in still further more particular worthy to be mentioned are those compounds of formula I, in which
R1 is bonded in the 8-position of the pyrrolo[2.1-a]isoquinoline ring, and is methoxy,
R2 is bonded in the 9-position of the pyrrolo[2.1-a]isoquinoline ring, and is methoxy,
R3 is hydrogen;
R4 is methyl,
R41 is hydrogen or methyl,
R5 is hydrogen,
R51 is hydrogen;
R6 is methyl;
R7 is naphthalen-1-yl, 4-hydroxy-3,5-dimethylphenyl, 3-dimethylamino-phenyl or quinolin-4-yl;
R8 is C(O)—OR9, in which
R9 is ethyl;
   and the salts, stereoisomers, hydrates and hydrates of the salts of these compounds.

In another facet of this invention (facet 2), compounds according to this invention more worthy to be mentioned are those compounds of formula I,
R1 is 1-4C-alkoxy, such as e.g. methoxy,
R2 is 1-4C-alkoxy, such as e.g. methoxy,
R3 is hydrogen,
   and none of R1 and R2 is bound to the 7- or 10-position of the pyrrolo[2.1-a]isoquinoline ring,
R4 is 1-4C-alkyl, such as e.g. methyl,
R41 is hydrogen,
R5 is hydrogen,
R51 is hydrogen,
R6 is 1-4C-alkyl, such as e.g. methyl,
R7 is Het2, R71- and/or R72- and/or R73-substituted phenyl, or naphthyl, in which
Het2 is a monocyclic or fused bicyclic 5- to 10-membered heteroaryl radical comprising one to three heteroatoms, each of which is selected from a group consisting of nitrogen, oxygen and sulfur,
R71 is hydroxyl, 1-4C-alkoxy or mono- or di-1-4C-alkylamino,
R72 is 1-4C-alkyl or 1-4C-alkoxy,
R73 is 1-4C-alkyl or 1-4C-alkoxy,
R8 is —C(O)—OR9, in which
R9 is 1-4C-allyl, such as e.g. ethyl,
   and the salts, stereoisomers, hydrates and hydrates of the salts of these compounds.

Also in the meaning of facet 2 of this invention, other compounds according to this invention more worthy to be mentioned are those compounds of formula I,
R1 is 1-4C-alkoxy, such as e.g. methoxy,
R2 is 1-4C-alkoxy, such as e.g. methoxy,
R3 is hydrogen,
   and none of R1 and R2 is bound to the 7- or 10-position of the pyrrolo[2.1-a]isoquinoline ring,
R4 is 1-4C-alkyl, such as e.g. methyl,
R41 is 1-4C-alkyl, such as e.g. methyl,
R5 is hydrogen,
R51 is hydrogen,
R6 is 1-4C-alkyl, such as e.g. methyl,
R7 is Het2, R71- and/or R72- and/or R73-substituted phenyl, or naphthyl, in which
Het2 is a monocyclic or fused bicyclic 5- to 10-membered heteroaryl radical comprising one to three heteroatoms, each of which is selected from a group consisting of nitrogen, oxygen and sulfur,
R71 is hydroxyl, 1-4C-alkoxy or mono- or di-1-4C-alkylamino,
R72 is 1-4C-alkyl or 1-4C-alkoxy,
R73 is 1-4C-alkyl or 1-4C-alkoxy,
R8 is —C(O)—OR9, in which
R9 is 1-4C-alkyl, such as e.g. ethyl,
   and the salts, stereoisomers, hydrates and hydrates of the salts of these compounds.

Also in the meaning of facet 2 of this invention, other compounds according to this invention more worthy to be mentioned are those compounds of formula I,
R1 is bonded in the position of the pyrrolo[2.1-a]isoquinoline ring, and is difluoromethoxy,
R2 is bonded in the 9-position of the pyrrolo[2.1-a]isoquinoline ring, and is methoxy,
R3 is hydrogen,
R4 is hydrogen,
R41 is hydrogen,
R5 is hydrogen,
R51 is hydrogen,
R6 is 1-4C-alkyl, such as e.g. methyl,
R7 is Het2, R71- and/or R72- and/or R73-substituted phenyl, or naphthyl, in which
Het2 is a monocyclic or fused bicyclic 5- to 10-membered heteroaryl radical comprising one to three heteroatoms, each of which is selected from a group consisting of nitrogen, oxygen and sulfur,
R71 is hydroxyl, 1-4C-alkoxy or mono- or di-1-4C-alkylamino,
R72 is 1-4C-alkyl or 1-4C-alkoxy,
R73 is 1-4C-alkyl or 1-4C-alkoxy,
R8 is —C(O)—OR9, in which
R9 is 1-4C-alkyl, such as e.g. ethyl,
   and the salts, stereoisomers, hydrates and hydrates of the salts of these compounds.

Also in the meaning of facet 2 of this invention, other compounds according to this invention more worthy to be mentioned are those compounds of formula I,
R1 is bonded in the position of the pyrrolo[2.1-a]isoquinoline ring, and is methoxy,
R2 is bonded in the 9-position of the pyrrolo[2.1-a]isoquinoline ring, and is difluoromethoxy,
R3 is hydrogen,
R4 is hydrogen,
R41 is hydrogen,
R5 is hydrogen,
R51 is hydrogen,
R6 is 1-4C-alkyl, such as e.g. methyl,
R7 is Het2, R71- and/or R72- and/or R73-substituted phenyl, or naphthyl, in which
Het2 is a monocyclic or fused bicyclic 5- to 10-membered heteroaryl radical comprising one to three heteroatoms, each of which is selected from a group consisting of nitrogen, oxygen and sulfur,
R71 is hydroxyl, 1-4C-alkoxy or mono- or di-1-4C-alkylamino,
R72 is 1-4C-alkyl or 1-4C-alkoxy,
R73 is 1-4C-alkyl or 1-4C-alkoxy,
R8 is —C(O)—OR9, in which
R9 is 1-4C-alkyl, such as e.g. ethyl,
   and the salts, stereoisomers, hydrates and hydrates of the salts of these compounds.

Also in the meaning of facet 2 of this invention, other compounds according to this invention more worthy to be mentioned are those compounds of formula I,
R1 is bonded in the 8-position of the pyrrolo[2.1-a]isoquinoline ring, and is 1-4C-alkoxy-2-4C-alkoxy, such as e.g. 2-methoxyethoxy,
R2 is bonded in the 9-position of the pyrrolo[2.1-a]isoquinoline ring, and is methoxy,
R3 is hydrogen,
R4 is hydrogen,
R41 is hydrogen,
R5 is hydrogen,
R51 is hydrogen,
R6 is 1-4C-alkyl, such as e.g. methyl,
R7 is Het2, R71- and/or R72- and/or R73-substituted phenyl, or naphthyl, in which
Het2 is a monocyclic or fused bicyclic 5- to 10-membered heteroaryl radical comprising one to three heteroatoms, each of which is selected from a group consisting of nitrogen, oxygen and sulfur,
R71 is hydroxyl, 1-4C-alkoxy or mono- or di-1-4C-alkylamino,
R72 is 1-4C-alkyl or 1-4C-alkoxy,
R73 is 1-4C-alkyl or 1-4C-alkoxy,
R8 is —C(O)—OR9, in which
R9 is 1-4C-alkyl, such as e.g. ethyl,
   and the salts, stereoisomers, hydrates and hydrates of the salts of these compounds.

Also in the meaning of facet 2 of this invention, other compounds according to this invention more worthy to be mentioned are those compounds of formula I,
R1 is halogen,
R2 is hydrogen,
R3 is 1-2C-alkoxy,
   particularly
R1 is bound to the 8-position and R3 is bound to the 9-position of the pyrrolo[2.1-a]isoquinoline ring, or
R1 is bound to the 9-position and R3 is bound to the 8-position of the pyrrolo[2.1-a]isoquinoline ring,
R4 is hydrogen,
R41 is hydrogen,
R5 is hydrogen,
R51 is hydrogen,
R6 is 1-4C-alkyl, such as e.g. methyl,
R7 is Het2, R71- and/or R72- and/or R73-substituted phenyl, or naphthyl, in which
Het2 is a monocyclic or fused bicyclic 5- to 10-membered heteroaryl radical comprising one to three heteroatoms, each of which is selected from a group consisting of nitrogen, oxygen and sulfur,
R71 is hydroxyl, 1-4C-alkoxy or mono- or di-1-4C-alkylamino,
R72 is 1-4C-alkyl or 1-4C-alkoxy,
R73 is 1-4C-alkyl or 1-4C-alkoxy,
R8 is —C(O)—OR9, in which
R9 is 1-4C-alkyl, such as e.g. ethyl,
   and the salts, stereoisomers, hydrates and hydrates of the salts of these compounds.

In yet another facet of this invention (facet 3), compounds according to this invention more worthy to be mentioned are those compounds of formula I,

either

R1 is halogen, 1-4C-alkyl, 1-4C-alkoxy, 3-7C-cycloalkoxy, 3-7C-cycloalkylmethoxy, 1-4C-alkoxy-2-4C-alkoxy, or completely or predominantly fluorine-substituted 1-4C-alkoxy,
R2 is 1-4C-alkoxy, and
R3 is 1-4C-alkoxy,
   or
R1 is 1-4C-alkoxy, 3-7C-cycloalkoxy, 3-7C-cycloalkylmethoxy, 1-4C-alkoxy-2-4C-alkoxy, or completely or predominantly fluorine-substituted 1-4C-alkoxy,
R2 is halogen, and
R3 is 1-4C-alkoxy;
R4 is hydrogen,
R41 is hydrogen,
R5 is hydrogen,
R51 is hydrogen;
R6 is 1-4C-alkyl;
R7 is Het2, R71- and/or R72- and/or R73-substituted phenyl, or naphthyl, in which
Het2 is a monocyclic or fused bicyclic 5- to 10-membered heteroaryl radical comprising one to three heteroatoms, each of which is selected from a group consisting of nitrogen, oxygen and sulfur,
R71 is hydroxyl, 1-4C-alkoxy or mono- or di-1-4C-alkylamino,
R72 is 1-4C-alkyl or 1-4C-alkoxy,
R73 is 1-4C-alkyl or 1-4C-alkoxy,
R8 is —C(O)—OR9, in which
R9 is 1-4C-alkyl, such as e.g. ethyl.
   and the salts, stereoisomers, hydrates and hydrates of the salts of these compounds.

Also in the meaning of facet 3 of this invention, compounds according to this invention in particular worthy to be mentioned are those compounds of formula I,

either

R1 is chlorine, fluorine, 1-4C-alkyl, 1-4C-alkoxy, 3-5C-cycloalkoxy, 3-5C-cycloalkylmethoxy, 1-4C-alkoxy-2-4C-alkoxy, or completely or predominantly fluorine-substituted 1-4C-alkoxy,
R2 is 1-4C-alkoxy, and
R3 is 1-4C-alkoxy,
   or
R1 is 1-4C-alkoxy, 3-5C-cycloalkoxy, 3-5C-cycloalkylmethoxy, 1-4C-alkoxy-2-4C-alkoxy, or completely or predominantly fluorine-substituted 1-4C-alkoxy,
R2 is chlorine or fluorine, and
R3 is 1-4C-alkoxy,
   and none of R1, R2 and R3 is bound to the 10-position of the pyrrolo[2.1-a]isoquinoline ring;
R4 is hydrogen,
R41 is hydrogen,
R5 is hydrogen,
R51 is hydrogen;
R6 is 1-4C-alkyl, such as e.g. methyl;
R7 is Het2,4-hydroxy-3,5-dimethylphenyl, 3-dimethylamino-phenyl, or naphthyl, in which
Het2 is a monocyclic or fused bicyclic 5- to 10-membered heteroaryl radical comprising one to three heteroatoms, each of which is selected from a group consisting of nitrogen, oxygen and sulfur,
R8 is —C(O)—OR9, in which
R9 is 1-4C-alkyl, such as e.g. ethyl,
   and the salts, stereoisomers, hydrates and hydrates of the salts of these compounds.

Also in the meaning of facet 3 of this invention, compounds according to this invention in more particular worthy to be mentioned are those compounds of formula I,
R1 is chlorine, fluorine, 1-4C-alkyl, 1-4C-alkoxy, 1-4C-alkoxy-2-4C-alkoxy, or completely or predominantly fluorine-substituted 1-4C-alkoxy,
R2 is 1-4C-alkoxy,
R3 is 1-4C-alkoxy, and none of R1, R2 and R3 is bound to the 10-position of the pyrrolo[2.1-a]isoquinoline ring;
R4 is hydrogen,
R41 is hydrogen,
R5 is hydrogen,
R51 is hydrogen;
R6 is 1-4C-alkyl, such as e.g. methyl;
R7 is Het2,4-hydroxy-3,5-dimethylphenyl, 3-dimethylamino-phenyl, or naphthyl, in which
Het2 is a monocyclic or fused bicyclic 5- to 10-membered heteroaryl radical comprising one to three heteroatoms, each of which is selected from a group consisting of nitrogen, oxygen and sulfur,
R8 is —C(O)—OR9, in which
R9 is 1-4C-alkyl, such as e.g. ethyl,
   and the salts, stereoisomers, hydrates and hydrates of the salts of these compounds.

As exemplary compounds according to this invention may be mentioned any compound selected from the group consisting of:
1. 2-(4-Hydroxy-3,5-dimethyl-phenyl)-8,9-dimethoxy-3,5,6-trimethyl-5,6-dihydro-pyrrolo[2,1-α]isoquinoline-1-carboxylic acid ethyl ester,
2. (6RS)-2-(4-Hydroxy-3,5-dimethyl-phenyl)-8,9-dimethoxy-3,6-dimethyl-5,6-dihydro-pyrrolo[2,1-a]isoquinoline-1-carboxylic acid ethyl ester,
3. (6RS)-8,9-Dimethoxy-3,6-dimethyl-2-(3,4,5-trimethoxy-phenyl)-5,6-dihydro-pyrrolo[2,1-a]isoquinoline-1-carboxylic acid ethyl ester,
4. 9-(1,1-Difluoro-methoxy)-2-(3-dimethylamino-phenyl)-8-methoxy-3-methyl-5,6-dihydro-pyrrolo[2,1-a]isoquinoline-1-carboxylic acid ethyl ester,
5. 9-(1,1-Difluoro-methoxy)-2-(4-hydroxy-3,5-dimethyl-phenyl)-8-methoxy-3-methyl-5,6-dihydro-pyrrolo[2,1-a]isoquinoline-1-carboxylic acid ethyl ester,
6. 9-(1,1-Difluoro-methoxy)-8-methoxy-3-methyl-2-(3,4,5-trimethoxy-phenyl)-5,6-dihydro-pyrrolo[2,1-a]isoquinoline-1-carboxylic acid ethyl ester,
7. 2-(4-Hydroxy-3,5-dimethyl-phenyl)-8,9,10-trimethoxy-3-methyl-5,6-dihydro-pyrrolo[2,1-a]isoquinoline-1-carboxylic acid ethyl ester,
8. 8-(1,1-Difluoro-methoxy)-9-methoxy-3-methyl-2-(3,4,5-trimethoxy-phenyl)-5,6-dihydro-pyrrolo[2,1-a]isoquinoline-1-carboxylic acid ethyl ester,
9. 8-(1,1-Difluoro-methoxy)-2-(4-hydroxy-3,5-dimethyl-phenyl)-9-methoxy-3-methyl-5,6-dihydro-pyrrolo[2,1-a]isoquinoline-1-carboxylic acid ethyl ester,
10. 8-(1,1-Difluoro-methoxy)-2-(3-dimethylamino-phenyl)-9-methoxy-3-methyl-5,6-dihydro-pyrrolo[2,1-a]isoquinoline-1-carboxylic acid ethyl ester,
11. 8,9-(1,1-Difluoro-methylenedioxy)-2-(3-dimethylamino-phenyl)-3-methyl-5,6-dihydro-pyrrolo[2,1-a]isoquinoline-1-carboxylic acid ethyl ester,
12. 8,9-(1,1-Difluoro-methylenedioxy)-2-(4-hydroxy-3,5-dimethyl-phenyl)-3-methyl-5,6-dihydro-pyrrolo[2,1-a]isoquinoline-1-carboxylic acid ethyl ester,
13. 8,9-(1,1-Difluoro-methylenedioxy)-3-methyl-2-(3,4,5-trimethoxy-phenyl)-5,6-dihydro-pyrrolo[2,1-a]isoquinoline-1-carboxylic acid ethyl ester,
14. 9-Chloro-2-(4-hydroxy-3,5-dimethyl-phenyl)-8-methoxy-3-methyl-5,6-dihydro-pyrrolo[2,1-a]isoquinoline-1-carboxylic acid ethyl ester,
15. 9-Chloro-8-methoxy-3-methyl-2-(3,4,5-trimethoxy-phenyl)-5,6-dihydro-pyrrolo[2,1-a]isoquinoline-1-carboxylic acid ethyl ester,
16. 9-Chloro-2-(3-dimethylamino-phenyl)-8-methoxy-3-methyl-5,6-dihydro-pyrrolo[2,1-a]isoquinoline-1-carboxylic acid ethyl ester,
17. 8-Chloro-2-(4-hydroxy-3,5-dimethyl-phenyl-9-methoxy-3-methyl-5,6-dihydro-pyrrolo[2,1-a]isoquinoline-1-carboxylic acid ethyl ester,
18. 2-(4-Hydroxy-3,5-dimethyl-phenyl)-9-methoxy-8-(2-methoxy-ethoxy)-3-methyl-5,6-dihydro-pyrrolo[2,1-a]isoquinoline-1-carboxylic acid ethyl ester,
19. 9-Methoxy-8-(2-methoxy-ethoxy)-3-methyl-2-(3,4,5-trimethoxy-phenyl)-5,6-dihydro-pyrrolo[2,1-a]isoquinoline-1-carboxylic acid ethyl ester,
20. 9-Methoxy-8-(2-methoxy-ethoxy-3-methyl-2-naphthalen-1-yl-5,6-dihydro-pyrrolo[2.1-a]isoquinoline-1-carboxylic acid ethyl ester,
21. 9-Fluoro-2-(4-hydroxy-3,5-dimethyl-phenyl)-8-methoxy-3-methyl-5,6-dihydro-pyrrolo[2,1-a]isoquinoline-1-carboxylic acid ethyl ester,
22. 2-(4-Hydroxy-3,5-dimethyl-phenyl)-8-methoxy-3-methyl-9-nitro-5,6-dihydro-pyrroio[2,1-a]isoquinoline-1-carboxylic acid ethyl ester,
23. 2-(4-Hydroxy-3,5-dimethyl-phenyl)-methoxy-3,9-dimethyl-5,6-dihydro-pyrrolo[2,1-a]isoquinoline-1-carboxylic acid ethyl ester,
24. 8,9-Dimethoxy-3-(2-methoxycarbonyl-ethyl)-6,6-dimethyl-2-quinolin-yl-5,6-dihydro-pyrrolo[2,1-a]isoquinoline-1-carboxylic acid ethyl ester,
25. 9-Amino-2-(4-hydroxy-3,5-dimethyl-phenyl)ethoxy-3-methyl-5,6-dihydro-pyrrolo[2,1-a]isoquinoline-1-carboxylic acid ethyl ester,
26. 1-[2-(4-Hydroxy-3,5-dimethyl-phenyl)-8,9-dimethoxy-3-methyl-5,6-dihydro-pyrralo[2,1-a]isoquinolin-1-yl-1-phenyl-methanone,
27. 4-(8,9-Dimethoxy-3-methyl-1-phenyl-5,6-dihydro-pyrrolo[2,1-a]isoquinolin-2-yl)-2,6-dimethyl-phenol,
28. 2-(4-Hydroxy-3,5-dimethyl-phenyl)-8,9-dimethoxy-3-methyl-5,6-dihydro-pyrrolo[2,1-a]isoquinoline-1-carboxylic acid cyclohexyl amide,
29. 1-[2-(4-Hydroxy-3,5-dimethyl-phenyl)-8,9-dimethoxy-3-methyl-5,6-dihydro-pyrrolo[2,1-a]isoquinolin-1-yl]-1-pyrrollidin-1-yl-methanone,
30. 2-(4-Hydroxy-3,5-dimethyl-phenyl)-8,9-dimethoxy-3-methyl-5,6-dihydro-pyrrolo[2,1-a]isoquinoline-1-carboxylic acid isopropylamide,
31. 2-(4-Hydroxy-3,5-dimethyl-phenyl)-8,9-dimethoxy-3-methyl-5,6-dihydro-pyrrolo[2,1-a]isoquinoline-1-carboxylic acid dimethylamide,
32. 2-(4-Hydroxy-3,5-dimethyl-phenyl)-8,9-dimethoxy-3-methyl-5,6-dihydro-pyrrolo[2,1-a]isoquinoline-1-carboxylic acid dimethylamide,
33. 2-(4-Hydroxy-3,5-dimethyl-phenyl)-8,9-dimethoxy-3-methyl-5,6-dihydro-pyrrolo[2,1-a]isoquinoline-1-carboxylic acid amide,
34. 2-(4-Hydroxy-3,5-dimethyl-phenyl)-8,9-dimethoxy-3-methyl-5,6-dihydro-pyrrolo[2,1-a]isoquinoline-1-carboxylic acid phenylamide,
35. 2-(4-Hydroxy-3,5-dimethyl-phenyl)-8,9-dimethoxy-3-methyl-5,6-dihydro-pyrrolo[2,1-a]isoquinoline-1-carboxylic acid ethylamide,
36. 2-(4-Hydroxy-3,5-dimethyl-phenyl)-8,9-dimethoxy-3-methyl-5,6-dihydro-pyrrolo[2,1-a]]isoquinoline-1-carboxylic acid sec-butylamide, and
37. 2-(4-Hydroxy-3,5-dimethyl-phenyl)-8,9-dimethoxy-3-methyl-5,6-dihydro-pyrrolo[2,1-a]isoquinoline-1-carboxylic acid cyclopropylamide;
   whereby as a more interesting exemplary compound according to this invention may be mentioned any compound selected from the group consisting of the following compounds specified by means of their Example numbers: 1, 2, 4, 5, 9, 10, 16, 18, 20, 21, 22, 25, 28, 30, 34 and 36; or the salts, stereoisomers, hydrates or hydrates of the salts thereof;

The compounds according to the present invention can be prepared, for example, in an art-known manner, or in a manner described and shown as follows, or as disclosed in WO 02/48144, WO 03/014115, WO 03/014116, WO 03/014117 or WO 03/051877, or as described byway of example in the following examples, or analogously or similarly thereto.

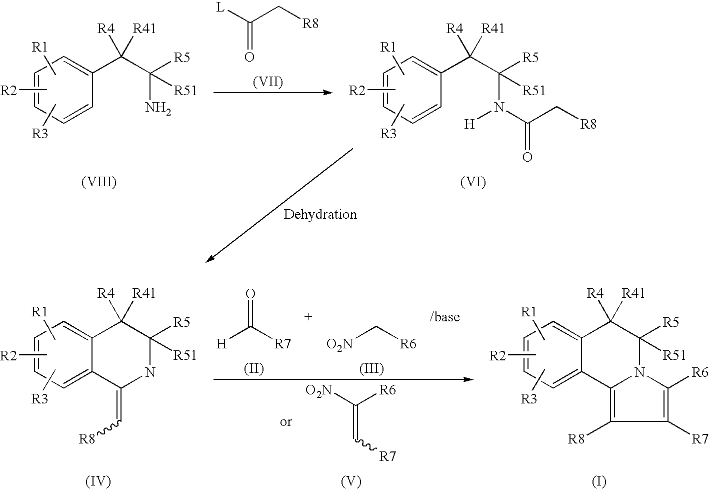

As shown in the scheme above, in a first reaction step compounds of formula VIII, in which R1, R2, R3, R4, R41, R5 and R51 have the meanings indicated above, are reacted with compounds of formula VII, in which R8 has the meanings indicated above and L is a suitable leaving group, for example chlorine or an acycloxy radical (e.g. the R8-CH₂—C(O)O— radical), to give in the presence of a suitable organic or inorganic base corresponding compounds of formula VI.

Alternatively, compounds of formula VI are also accessible from compounds of formula VIII, in which R1, R2, R3, R4, R41, R5 and R51 have the meanings indicated above, and compounds of formula VII, in which R8 has the meanings indicated above and L is hydroxyl, by reaction with amide bond linking reagents known to the person skilled in the art. Exemplary amide bond linking reagents known to the person skilled in the art which may be mentioned are, for example, the carbodiimides (e.g. dicyclohexylcarbodiimide or, preferably, 1-ethyl-3-(3-dimethylaminopropyl)carbodiimide hydrochloride), azodicarboxylic acid derivatives (e.g. diethyl azodicarboxylate), uronium salts [e.g. 0-(benzotriazol-1-yl)-N,N,N′,N′-tetramethyluronium tetrafluoroborate or O-(benzotriazol-1-yl)-N,N,N′,N′-tetramthyl-uronium-hexafluorophosphate] and N,N′-carbonyldiimidazole. In the scope of this invention preferred amide bond linking reagents are uronium salts and, particularly, carbodiimides, preferably, 1-ethyl-3-(3-dimethylaminopropyl)carbodiimide hydrochloride.

Said reactions are carried out under conditions known to the person skilled in the art or as described exemplarily in the following examples.

As shown in the next step, compounds of the formula IV, in which R1, R2, R3, R4, R41, R5, R51 and R8 have the meanings indicated above, can be obtained by cyclocondensation of corresponding compounds of the formula VI. Said cyclocondensation reaction is carried out in a manner habitual per se to the person skilled in the art or as described by way of example in the following examples, according to Bischler-Napieralski (e.g. as described in J. Chem. Soc., 1958, 4280-4282) in the presence of a suitable condensing or dehydrating agent, such as, for example, polyphosphoric add, phosphorus pentachloride, phosphorus pentoxide or phosphorus oxychloride, in a suitable inert solvent, e.g. in a chlorinated hydrocarbon such as chloroform, or in a cyclic hydrocarbon such as toluene or xylene, or another inert solvent such as acetonitrile, or without further solvent using an excess of condensing agent, at reduced temperature, or at room temperature, or at elevated temperature or at the boiling temperature of the solvent or condensing agent used.

Compounds of formula IV are converted either with compounds of formulae II, in which R7 has the meanings given above, and III, in which R6 is 1-6C-alkyl or 1-4C-alkyl substituted by 1-4C-alkoxycarbonyl, or with compounds of formula V, in which R7 has the meanings given above and R6 is 1-6C-alkyl or 1-4C-alkyl substituted by 1-4C-alkoxycarbonyl, optionally in a one pot synthesis and suitably in the presence of an inorganic or organic base (in particular a cyclic amine, e.g. piperidine) into the corresponding compounds of formula I.

Said conversion can be carried out as known to the skilled person or as described in the following examples or analogously or similarly thereto.

Compounds of formulae VIII, VII, III and II are commercially available or can be obtained in a manner known to the skilled person from his/her expert knowledge and/or from literature.

Compounds of formula V are known or are accessible by reaction of compounds of formula II with compounds of formula III in the presence of a suitable organic or inorganic base in a manner customary per se to the skilled person.

Compounds of formula I obtained can be converted into further compounds of formula I by methods known to one of ordinary skill in the art. More specifically, for example, from compounds of the formula I, in which
a.) R8, R61, R71, R74 or R76 are an ester group, the corresponding adds can be obtained by acidic or, particularly, alkaline hydrolysis;
b.) R8 is an ester or carboxyl group, the corresponding amides can be obtained by amidification reactions;
c.) R6 is 1-4C-alkyl, particularly methyl, the corresponding halogenated, preferably chlorinated, groups can be obtained by halogenation reaction, particularly by reaction with a chlorination reagent such as sulfuryl chloride, thionyl chloride or N-chlorosuccinimide;
d.) R6 is 1-4C-alkyl substituted by halogen, the corresponding derivatized 1-4C-alkyl radicals substituted by 1-4C-alkoxy, hydroxyl, halogen or —N(R611)R612 can be obtained by nucleophilic substitution reactions with suitable nucleophiles;
e.) R6 is 1-4C-alkyl substituted by hydroxyl, the corresponding derivatized 1-4C-alkyl radicals substituted by 1-4C-alkoxycarbonyl can be obtained by oxidation and esterification reactions under suitable conditions;
f.) R6 is methyl, the corresponding oxidized forms thereof (e.g. the hydroxymethyl or formyl radicals) can be obtained stepwise or directly by selective oxidation reactions (e.g. with the aid of manganese dioxide to obtain the formyl radicals);
g.) R6 is formyl, the corresponding aminated compounds can be obtained by reductive amination reaction;
h.) R6 is hydroxymethyl, the corresponding fluorine compounds can be obtained by fluorination reaction;
i.) R6 is methyl, the corresponding amino compounds can be obtained by nitration reaction and subsequential reduction of the nitro compounds obtained.

The methods mentioned under a.) to i.) are expediently carried out analogously to the methods known to the person skilled in the art or as described by way of example in the following examples. It is moreover known to the person skilled in the art that if there are a number of reactive centers on a starting or intermediate compound it may be necessary to block one or more reactive centers temporarily by protective groups in order to allow a reaction to proceed specifically at the desired reaction center. A detailed description for the use of a large number of proven protective groups is found, for example, in “Protective Groups in Organic Synthesis” by T. Greene and P. Wuts (John Wiley & Sons, Inc. 1999, 3^{rd} Ed.) or in “Protecting Groups (Thieme Foundations Organic Chemistry Series N Group” by P. Kocienski (Thieme Medical Publishers, 2000).

The isolation and purification of the substances according to the invention is carried out in a manner known per se, e.g. by distilling off the solvent in vacuo and recrystallizing the resulting residue from a suitable solvent or subjecting it to one of the customary purification methods, such as, for example, column chromatography on suitable support material.

Salts are obtained by dissolving the free compound in a suitable solvent (e.g. a ketone, such as acetone, methyl ethyl ketone or methyl isobutyl ketone, an ether, such as diethyl ether, tetrahydrofuran or dioxane, a chlorinated hydrocarbon, such as methylene chloride or chloroform, or a low molecular weight aliphatic alcohol such as ethanol or isopropanol) which contains the desired acid or base, or to which the desired acid or base is then added. The salts are obtained by filtering, reprecipitating, precipitating with a nonsolvent for the addition salt or by evaporating the solvent. Salts obtained can be converted by alkalization or by acidification into the free compounds, which in turn can be converted into salts. In this way, pharmacologically intolerable salts can be converted into pharmacologically tolerable salts.

The person skilled in the art knows on the basis of his/her knowledge and on the basis of those synthesis mutes, which are shown and described within the description of this invention, how to find other possible synthesis routes for compounds of the formula I. All these other possible synthesis routes are also part of this invention.

Having described the invention in detail, the scope of the present invention is not limited only to those described characteristics or embodiments. As will be apparent to persons skilled in the art, modifications, variations and adaptations to the described invention can be made on the base of the disclosure (e.g. the explicite, implicite or inherent disclosure) of the present invention without departing from the spirit and scope of this invention.

The following examples serve to illustrate the invention in greater detail without restricting it. Likewise, further compounds of the formula I, whose preparation is not explicitly described, can also be prepared in an analogous manner or in a manner familiar per se to the person skilled in the art using customary process techniques.

In the examples, m.p. stands for melting point, h for hour(s), min for minutes, conc. for concentrated, said. for saturated, MS for mass spectrum, M for molecular ion.

The compounds mentioned in the examples as well as their salts and stereoisomers are a preferred subject of the invention.

### EXAMPLES

Final Products

### 1. 2-(4-Hydroxy-3,5-dimethyl-phenyl-8,9-dimethoxy-3,5,6-trimethyl-5,6-dihydro-pyrrolo[2,1-a]isoquinolin-1-carboxylic acid ethyl ester

Analogously to a procedure described by Meyer in Liebigs Ann. Chem. 1981, 9, 1534-1544, (6,7-dimethoxy-4,4-dimethyl-3,4-dihydro-2H-isoquinolin-1-ylidene)-acetic acid ethyl ester (compound A8) is reacted with nitro ethane and 4-hydroxy-3,5-dimethyl benzaldehyde to afford 2-(4-hydroxy-3,5-dimethyl-phenyl)-8,9-dimethoxy-3,6,6-trimethyl-5,6-dihydro-pyrrolo[2,1-a]isoquinoline-1-carboxylic acid ethyl ester as a colorless solid of m.p. 200-202° C. The mass spectrum shows the molecular peak M+H at 464.3 Da.

The following examples (Examples 2-24) can be prepared in analogy to example 1 using the appropriate starting compound selected from the group consisting of the compounds A1 to A14. All aldehydes used are commercially available or can be prepared in analogy to published procedures. If nitro propane or 4-nitro butyric acid methyl ester is used instead of nitroethane, 3-ethyl-5,6-dihydro-pyrollo[2,1-a]isoquinolines and 3-(8,9-dimethoxy-5,6-dihydro-pyrrolo[2,1-a]isoquinolin-3-yl)propionic methyl esters, respectively are obtained.

### 2. (6RS)-2-(4-Hydroxy-3,5-dimethyl-phenyl)-8,9-dimethoxy-3,6-dimethyl-5,6-dihydro-prrolo[2,1-a]isoquinoline-1-carboxylic acid ethyl ester

MS (M+H)=450.1; m.p.=191-194° C.

### 3. (6RS)-8,9-Dimethoxy-3,6-dimethyl-2-(3,4,5-trimethoxy-phenyl)-5,6-dihydro-pyrroio[2,1-a]isoquinoline-1-carboxylic acid ethyl ester

MS (M+H)=496.0; m.p.=150° C.

### 4. 9-(1,1-Difluoro-methoxy)-2-(3-dimethylamino-phenyl)-8-methoxy-3-methyl-5,6-dihydro-pyrrolo[2,1-a]isoquinoline-1-carboxylic acid ethyl ester

MS (M+H)=470.8; m.p.=107-110° C.

### 5. 9-(1,1-Difluoro-methoxy)-2-(4-hydroxy-3,5-dimethyl-phenyl)-8-methoxy-3-methyl-5,6-dihydro-pyrrolo[2,1-a]isoquinoline-1-carboxylic acid ethyl ester

MS (M+H)=471.8; m.p.=152-155° C.

### 6. 9-(1,1-Difluoro-methoxy-methoxy-methyl-2-(3,4,5-trimethoxy-phenyl) 5,6-dihydro-pyrrolo[2,1-a]isoquinoline-1-carboxylic acid ethyl ester

MS (M+H)=517.8; m.p.=138-141° C.

### 7. 2-(4-Hydroxy-3,5-dimethyl-phenyl)-8,9,10-trimethoxy-3-methyl-5,6-dihydro-pyrrolo[2,1-a]isoquinoline-1-carboxylic acid ethyl ester

MS (M+H)=466.1; m.p.=246-251° C.

### 8. 8-(1,1-Difluoro-methoxy)-9-methoxy-3-methyl-2-(3,4,5-trimethoxy-phenyl)-5,6-dihydro-pyrrolo[2,1-a]isoquinoline-1-carboxylic acid ethyl ester

MS (M+H)=517.7; m.p.=155° C.

### 9. 8-(1,1-Difluoro-methoxy)-2-(4-hydroxy-3,5-dimethyl-phenyl)-9-methoxy-3-methyl-5,6-dihydro-pyrrolo[2,1-a]isoquinoline-1-carboxylic acid ethyl ester

MS (M+H)=471.7; m.p.=126-128° C.

### 10. 8-(1,1-Difluoro-methoxy)-2-(dimethylamino-phenyl)-9-methoxy-3-methyl-5,6-dihydro-pyrrolo[2,1-a]isoquinoline-1-carboxylic acid ethyl ester

MS (M+H)=470.7; m.p.=118-120° C.

### 11. 8,9-(1,1-Difluoro-methylenedioxy)-2-(3-dimethylamino-phenyl)-3-methyl-5,6-dihydro-pyrrolo[2,1-a]isoquinoline-1-carboxylic acid ethyl ester

MS (M+H)=454.8; m.p.=136-139° C.

### 12. 8,9-(1,1-Difluoro-methylenedioxy)-2-(4-hydroxy-3,5-dimethyl-phenyl)-3-methyl-5,6-dihydro-pyrrolo[2,1-a]isoquinoline-1-carboxylic acid ethyl ester

MS (M+H)=455.6; m.p.=176-180° C.

### 13. 8,9-(1,1-Difluoro-methylenedioxy)-3-methyl-2-(3,4,5-trimethoxy-phenyl)-5,6-dihydro-pyrrolo[2,1-a]isoquinoline-1-carboxylic acid ethyl ester

MS (M+H)=501.7; m.p.=138-141° C.

### 14. 9-Chloro-2-(4-hydroxy-3,5-dimethyl-phenyl)-8-methoxy-3-methyl-5,6-dihydro-pyrroio[2,1-a]isoquinoline-1-carboxylic acid ethyl ester

MS (M+H)=470.7; m.p.=118-120° C.

### 15. 9-Chloro-8-methoxy-3-methyl-2-(3,4,5-trimethoxy-phenyl)-5,6-dihydro-pyrrolo[2,1-a]isoquinoline-1-carboxylic acid ethyl ester

MS (M+H)=485.6; m.p.=172-174° C.

### 16. 9-Chloro-2-(3-dimethylamino-phenyl)-8-methoxy-3-methyl-5,6-dihydro-pyrrolo[2,1-a]isoquinoline-1 caboxylic acid ethyl ester

MS (M+H)=438.9; m.p.=133-135° C.

### 17. 8-Chloro-2-(4-hydroxy-3,5-dimethyl-phenyl)-9-methoxy-3-methyl-5,6-dihydro-pyrrolo[2,1-a]isoquinoline-1-carboxylic acid ethyl ester

MS (M+H)=439.7; m.p.=167-169° C.

### 18. 2-(4-Hydroxy-3,5-dimethyl-phenyl)-9-methoxy-8-(2-methoxy-ethoxy)-3-methyl-5,6-dihydro-pyrrolo[2,1-a]isoquinoline-1-carboxylic acid ethyl ester

MS (M+H)=480.2; m.p.=169-171° C.

### 19. 9-Methoxy-8-(2-methoxy-ethoxy)-3-methyl-2-(3,4,5-trimethoxy-phenyl)-5,6-dihydro-pyrrolo[2,1-a]isoquinoline-1-carboxylic acid ethyl ester

MS (M+H)=526.0; m.p.=152-154° C.

### 20. 9-Methoxy-8-(2-methoxy-ethoxy)-3-methyl-2-naphthalen-1-yl-5,6-dihydro-pyrrolo[2,1-a]isoquinoline-1-carboxylic acid ethyl ester

MS (M+H)=486.2; m.p.=126-128° C.

### 21. 9-Fluoro-2-(4-hydroxy-3,5-dimethyl-phenyl-ethoxy-3-methyl-5,6-dihydro-pyrrolo[2,1-a]isoquinoline-1-carboxylic acid ethyl ester

MS (M+H)=423.6; m.p.=180-182° C.

### 22. 2-(4-Hydroxy-3,5-dimethyl-phenyl)-8-methoxy-3-methyl-9-nitro-5,6-dihydro-pyrrolo[2,1-a]isoquinoline-1-carboxylic acid ethyl ester

MS (M+H)=450.7; m.p.=209-211° C.

### 23. 2-(4-Hydroxy-3,5-dimethyl-phenyl)-8-methoxy-3,9-dimethyl-5,6-dihydro-pyrrolo[2,1-a]isoquinoline-1-carboxylic acid ethyl ester

MS (M+H)=420.0; m.p.=179-181° C.

### 24. 8,9-Dimethoxy-3-(2-methoxycarbonyl-ethyl)-6,6-dimethyl-2-quinolin-4-yl-5,6-dihydro-pyrrolo[2,1-a]isoquinoline-1-carboxylic acid ethyl ester

MS (M+H)=543.4; oil

### 25. 9-Amino-2-(4-hydroxy-3,5-dimethyl-phenyl)-8-methoxy-3-methyl-5,6-dihydro-pyrrolo[2,1-a]isoquinoline-1-carboxylic acid ethyl ester

A suspension of 200 mg (4.43 mmol) of 2-(4-hydroxy-3,5-dimethyl-phenyl)-8-methoxy-9-nitro-3-methyl-5,6-dihydro-pyrrolo[2,1-a]isoquinoline-1-carboxylic acid ethyl ester (Example 22) and 100 mg of Pd/C (10%) catalyst in 30 ml of ethanol is placed into an apparatus parr. The bottle is filled with hydrogen at an initial pressure of 30 psi and shaked during 3 hours. The solution is filtered on celite and washed with ethanol and ethyl acetate. After evaporation of the solvents, the residue is purified by chromatography on silica gel eluting with ethyl acetate/petroleum spirit (5:5) to afford 110 mg (59%) of the title compound as a beige solid of m.p. 104-106° C. The mass spectrum shows the molecular peak M+H at 420.8 Da.

### 26. 1-[2-(4-Hydroxy-3,5-dimethyl-phenyl)-8,9-dimethoxy-methyl-5,6-dihydro-pyrrolo[2,1-a]isoquinolin-1-yl]-1-phenyl-methanone

Analogously to the procedure described for Example 1,2-(6,7-dimethoxy-3,4-dihydro-2H-isoquinolin-1-ylidene)-1-phenyl-ethanone (compound A13) is reacted with nitro ethane and 4-hydroxy-3,5-dimethyl benzaldehyde to afford 1-[2-(4-hydroxy-3,5-dimethyl-phenyl)-8,9-dimethoxy-3-methyl-5,6-dihydro-pyrrolo[2,1-a]isoquinolin-1-yl]-1-phenyl-methanone as a colorless solid of m.p. 194-196° C. The mass spectrum shows the molecular peak M+H at 467.6 Da.

### 27. 4-(8,9-Dimethoxy-3-methyl-1-phenyl-5,6-dihydro-pyrrolo[2,1-a]isoquinolin-2-yl)-2,6-dimethyl-phenol

Analogously to the procedure described for Example 1,1-benzylidene-6,7-dimethoxy-1,2,3,4 tetrahydro-isoquinoline (compound A14) is reacted with nitro ethane and 4-hydroxy-3,5-dimethyl benzaldehyde to afford 4-(8,9-dimethoxy-3-methyl-1-phenyl-5,6-dihydro-pyrrolo[2,1-a]isoquinolin-2-yl)-2,6-dimethyl-phenol as a colorless solid of m.p. 210-214° C. The mass spectrum shows the molecular peak M+H at 439.6 Da.

### 28. 2-(4-Hydroxy-3,5-dimethyl-phenyl)-8,9-dimethoxy-3-methyl-5,6-dihydro-pyrrolo[2,1-a]isoquinoline-1-carboxylic acid cyclohexyl amide

To a solution of 190 μl (1.65 mmol) of cyclohexyl amine in 2 ml of toluene at 0° C. is added dropwise 970 μl (1.92 mmol) of a 2.0 M trimethylaluminum solution in toluene. The reaction mixture is stirred at room temperature for 1 hour and a solution of 240 mg (550 μmol) of 2-(4-hydroxy-3,5-dimethyl-phenyl)-8,9-dimethoxy-3-methyl-5,6-dihydro-pyrrolo[2,1-a]isoquinoline-1-carboxylic acid ethyl ester (Example 1) dissolved in 4 ml of tetrahydrofurane and 2 ml of toluene is added dropwise. The resulting mixture is stirred in a sealed tube at 110° C. for 16 hours (reaction followed by TLC analysis). The reaction mixture is cooled to room temperature and 5 N aqueous sodium hydroxide solution is added slowly. The mixture is diluted with water and extracted twice with ethyl acetate. The combined organic phases are dried over magnesium sulfate and concentrated. The residue is purified by chromatography on silica gel eluting with ethyl acetate/petroleum spirit (5:5) and then with ethyl acetate to afford 110 mg (41%) of the title compound as a white solid of m.p. 273-276° C. The mass spectrum shows the molecular peak M+H at 488.6 Da.

The following examples (Examples 29-37) can be prepared in analogy to Example 28. All amines used are commercially available. If ammonia chloride is used instead of cyclohexyl amine, the free amide is obtained.
29. 1-[2-(4-Hydroxy-3,5-dimethyl-phenyl)-8,9-dimethyl-5,6-dihydro-pyrrolo[2,1-a]isoquinolin-1-yl]-pyrrollidin-1-yl-methanone

MS (M+H)=460.6; m.p.=216-218° C.

### 30. 2-(4-Hydroxy-3,5-dimethyl-phenyl)-8,9-dimethoxy-3-methyl-5,6-dihydpyrrolo[2,1-a]isoquinoline-1-carboxylic acid isopropylamide

MS (M+H)=448.9; m.p.=233-235° C.

### 31. 2-(4-Hydroxy-3,5-dimethyl-phenyl)-8,9-dimethoxy-3-methyl-5,6-dihydro-pyrrolo[2,1-a]isoquinoline-1-carboxylic acid dimethylamide

MS (M+H)=434.5; m.p.=259-261° C.

### 32. 2-(4-Hydroxy-3,5-dimethyl-phenyl)-8,9-dimethoxy-3-methyl-5,6-dihydro-pyrrolo[2,1-a]isoquinoline-1-carboxylic acid methylamide

MS (M+H)=421.3; m.p.=281-283° C.

### 33. 2-(4-Hydroxy-3,5-dimethyl-phenyl)-8,9-dimethoxy-3-methyl-5,6-dihydro-pyrrolo[2,1-a]isoquinoline-1-carboxylic acid amide

MS (M+H)=407.2; m.p.=229-231° C.

### 34. 2-(4-Hydroxy-3,5-dimethyl-phenyl)-8,9-dimethoxy-3-methyl-5,6-dihydro-pyrrolo[2,1-a]isoquinoline-1-carboxylic acid phenylamide

MS (M+H)=482.6; m.p.=271-273° C.

### 35. 2-(4-Hydroxy-3,5-dimethyl-phenyl)-8,9-dimethoxy-3-methyl-5,6-dihydro-pyrrolo[2,1-a]isoquinoline-1-carboxylic acid ethylamide

MS (M+H)=435.9; m.p.=242-244° C.

### 36. 2-(4-Hydroxy-3,5-dimethyl-phenyl)-8,9-dimethoxy-3-methyl-5,6-dihydro-pyrrolo[2,1-a]isoquinoline-1-carboxylic acid sec-butylamide

MS (M+H)=464; m.p.=238-240° C.

### 37. 2-(4-Hydroxy-3,5-dimethyl-phenyl)-8,9-dimethoxy-3-methyl-5,6-dihydro-pyrrolo[2,1-a]isoquinoline-1-carboxylic acid cyclopropylamide

MS (M+H)=448.1; m.p.=254-256° C.

Starting Compounds

### A1 [7-Methoxy-6-(2-methoxy-ethoxy)-3,4-dihydro-2H-isoquinolin-1-ylidene)-acetic acid ethyl ester

The title compound can be obtained by a Bischler-Napieralski reaction (Ber. 1893, 26, 1903) using N-{2-[4-methoxy-3-(2-methoxy-ethoxy)phenyl]-ethyl}-malonamic acid ethyl ester (compound B1) as the starting material.

MS (M+H)=237.2; m.p.=79-81° C.

The following 3,4-Dihydro-1-(2H)-isoquinolinylidene-derivatives A2 to A13 can be prepared according an analogous procedure using the appropriate starting compound selected from the group consisting of the compounds B2 to B13:
A2 (7-Difluoromethoxy-6-methoxy-3,4-dihydro-2H-isoquinolin-1-ylidene)-acetic acid ethyl ester
A3 (6-Difluoromethoxy-7-methoxy-3,4-dihydro-2H-isoquinolin-1-ylidene)-acetic acid ethyl ester
A4 (2,2-Difluoro-7,8-dihydro-6H-[1,3]dioxolo[4,5-g]isoquinolin-5-ylidene)-acetic acid ethyl ester
A5 (7-Chloro-6-methoxy-3,4-dihydro-2H-isoquinolin-1-ylidene)-acetic acid ethyl ester
A6 (6-Chloro-7-methoxy-3,4-dihydro-2H-isoquinolin-1-ylidene)-acetic acid ethyl ester
A7 (4RS)-(6,7-Dimethoxy-4-methyl-3,4-dihydro-2H-isoquinolin-1-ylidene)-acetic acid ethyl ester
A8 (6,7-Dimethoxy-4,4-dimethyl-3,4-dihydro-2H-isoquinolin-1-ylidene)-acetic acid ethyl ester
A9 (6,7,8-Trimethoxy-3,4-dihydro-2H-isoquinolin-1-ylidene)-acetic acid ethyl ester
A10 (6-Methoxy-7-methyl-3,4-dihydro-2H-isoquinolin-1-ylidene)-acetic acid ethyl ester
A11 (6-Methoxy-7-nitro-3,4-dihydro-2H-isoquinolin-1-ylidene)-acetic acid ethyl ester
A12 (7-Fluoromethoxy-3,4-dihydro-2H-isoquinolin-1-ylidene)-acetic acid ethyl ester
A13 2-(6,7-Dimethoxy-3,4-dihydro-2H-isoquinolin-1-ylidene)-1-phenyl-ethanone

The compound A13 can be prepared analogously to the above-described synthesis of compound A1 using the starting compound B13.
A14 1-Benzylidene-6,7-dimethoxy-1,2,3,4-tetrahydro-isoquinoline

The compound A14 is commercially available.

### B1 N-{2-[4-methoxy-3-(2-methoxy-ethoxy)-phenyl]-ethyl}-malonamic acid ethyl ester

N{2-[4-methoxy-3-(2-methoxy-ethoxy)-phenyl]-ethyl}-malonamic acid ethyl ester can be prepared by a reaction of 2-[4-Methoxy-3-(2-methoxy-ethoxy)-phenyl)-ethylamine (compound C1) with ethyl maloyl chloride in analogy to procedures in the literature (e.g. Benovsky et al., Tetrahedron Lett. 1997, 38, 8475-8478).

MS (M+H)=3402; m.p.=70° C.

The following amides B2 to B12 can be synthesized according an analogous procedure:
B2 N-{2-[4-(1,1-Difluoro-methoxy)-3-methoxy-phenyl]-ethyl}malonamic acid ethyl ester
B3 N-{2-[3-(1,1-Difluoro-methoxy)-4-methoxy-phenyl]-ethyl}-malonamic acid ethyl ester
B4 N-[2-(2,2-Difluoro-benzo[1,3]dioxol-5-yl)-ethyl]-malonamic acid ethyl ester
B5 N-[2-(4Chloro-3-methoxy-phenyl)-ethyl]-malonamic acid ethyl ester
B6 N-[2-(3-Chloro-4-methoxy-phenyl)-ethyl]-malonamic acid ethyl ester
B7 N-[(RS)-2-(3,4-Dimethoxy-phenyl)-propyl]-malonamic acid ethyl ester
B8 N-[2-(3,4-Dimethoxy-phenyl)-2-methyl-propyl]-malonamic acid ethyl ester
B9 N-[2-(3,4,5-Trimethoxy-phenyl)-ethyl]-malonamic acid ethyl ester
B10 N-[2-(3-Methoxy-4-methyl-phenylkthyl]-malonamic acid ethyl ester
B11 N-[2-(3-Methoxy-4-nitro-phenyl)-ethyl]-malonamic acid ethyl ester
B12 N-[2-(4-Fluoro-3-methoxy-phenyl)-ethyl]-malonamic acid ethyl ester
B13 N-[2-(3,4-Dimethoxy-phenyl)-ethyl]-3-oxo-3-phenyl-propionamide

To a solution of 1.10 g (6.07 mmol) of 2-(3,4-dimethoxy-phenyl)-ethylamine in toluene (6 mL) at 0° C. is added dropwise 3.78 mL (7.57 mmol) of a trimethylaluminum 2.0 M solution in toluene. The reaction mixture is stirred at room temperature during 1 hour and a solution of 0.53 mL (3.03 mmol) of ethyl benzoylacetate in toluene (4 ml) is added dropwise. The resulting mixture is stirred in a sealed tube at 100° C. during 16 hours (reaction followed by TLC analysis). The reaction mixture is cooled to room temperature and 5 N aqueous solution of sodium hydroxide is slowly added. The mixture is diluted with water and extracted twice with ethyl acetate. The combined organic phases are dried over magnesium sulfate and concentrated. The residue is purified by chromatography on silica gel eluting with ethyl acetate to afford 680 mg (68%) of the title compound as a yellow oil. MS (M+H)=227.7

The appropriate starting compounds for the preparation of the compounds B1 to B13 are commercially available, or can be prepared as described below in the synthesis of the compounds C1 to C3 or analogously or similarly thereto, or can be obtained in analogy to published procedures, e.g. the substituted 2-phenethyl amines can be prepared starling from the corresponding benzaldehydes (see also Shepard et al., J. Org. Chem. 1952, 17, 568).

### C1 2-[4-Methoxy-3-(2-methoxy-ethoxy)-phenyl]-ethylamine

2-[4-Methoxy-3-(2-methoxy-ethoxy)-phenyl]-ethylamine can be prepared by alkylation of 4-methoxy-3-hydroxy benzaldehyde with 2-bromomethyl ethyl ether (analogous to a procedure by Ashton et al., J. Med. Chem. 1994, 37, 1696-1703), followed by a sequence described by Shepard et al. in J. Org. Chem. 1952, 17, 568.

MS (M+H)=226.0

### C2 2-[4-(1,1-Difluoro-methoxy)-3-methoxy-phenyl]-ethylamine

2-[4-(1,1-Difluoro-methoxy)-3-methoxy-phenyl]-ethylamine can be prepared by difluoromethylation of 4-hydroxy-3-methoxy benzaldehyde with chloro difluoro methane according to a procedure published by Amschler et al. (WO97/28131), followed by a sequence described by Shepard et al. in J. Org. Chem. 1952, 17, 568.

MS (M+H)=217.6

### C3 3-[4-(1,1-Difluoro-methoxy)-2-methoxy-phenyl]-ethylamine

3-[4-(1,1-Difluoro-methoxy)-2-methoxy-phenyl]-ethylamine was prepared by difluoromethylation of 3-hydroxy-4-methoxy benzaldehyde with chloro difluoro methane according to a procedure published by Amschler et al. (WO97/28131), followed by a sequence described by Shepard et al. in J. Org. Chem. 1952, 17, 568.

MS (M+H)=217.7

Commercial Utility

Commercial Applicability

The compounds according to the invention have miscellaneous valuable pharmacological properties which make them commercially utilizable.

The compounds according to the invention therefore can be employed as therapeutic agents for the treatment and prophylaxis of diseases in human and veterinary medicine.

Various diseases are caused by limitless replicative potential and aberrant cell proliferation (“hyperproliferation”) as well as evasion from apoptosis. These diseases include benign hypoplasia like that of the prostate (“BPH”) or colon epithelium. Most importantly these diseases include malignant neoplasias commonly described as cancer and characterized by tumor cells finally metastasizing into distinct organs or tissues. Malignant neoplasia include solid and hematological tumors. Solid tumors are exemplified by tumors of the breast, bladder, bone, brain, central and peripheral nervus system, colon, endocrine glands (eg thyroid and adrenal cortex), esophagus, endometrium, germ cells, head and neck, kidney, liver, lung, larynx and hypopharynx, mesothelioma, sarcoma, ovary, pancreas, prostate, rectum, renal, small intestine, soft tissue, testis, stomach, skin, ureter, vagina and vulva. Malignant neoplasias include inherited cancers exemplified by retinoblastoma and Wilms tumor. In addition, malignant neoplasia include primary tumors in said organs and corresponding secondary tumors in distant organs (“tumor metastases”). Hematological tumors are exemplified by aggressive and indolent forms of leukemia and lymphoma, namely non-Hodgkins disease, chronic and acute myeloid leukemia (CML/AML), acute lymphoblastic leukemia (ALL), Hodgkins disease, multiple myeloma and T-cell lymphoma. Also included are myelodysplastic syndrome, plasma cell neoplasia, paraneoplastic syndromes, cancers of unknown primary site as well as AIDS related malignancies.

Compounds according to the present invention will commercially applicable for treatment of the diseases of benign and malignant behavior as described before, such as e.g. cancer.

Neoplastic cell proliferation might effect normal cell behaviour and organ function. For example the formation of new blood vessels, a process described as neovascularization, is induced by tumors or tumor metastases. Compounds according to this invention will commercially applicable for treatment of pathophysiological relevant processes caused by benign or neoplastic cell proliferation, such as but not limited to neovascularization by unphysiological proliferation of vascular endothelial cells.

Drug resistance is of particular importance for the frequent failure of standard cancer therapeutics. This drug resistance is caused by various cellular and molelcular mechanisms like overexpression of drug efflux pumps or mutation within the cellular target protein. The commercial applicability of the compounds according to this invention is not limited to 1^{st} line treatment of patients. Patients with resistance to defined cancer chemotherapeutics or target specific anti-cancer drugs (2^{nd} or 3^{rd} line treatment) are also amenable for treatment with the compounds according to this invention.

In a facet of the present invention, the expression “cancer” includes solid tumors as well as leukemia, lymphoma and myeloma. Among solid tumors, preferred indications are malignancies of the lung, breast, pancreas, brain, prostate and ovar. Within this facet, the invention relates to a method for treating mammals, including humans, which/who are suffering from cancer. The method is characterized by the fact that a therapeutically effective and pharmacologically tolerated quantity of one or more of the compounds according to the invention is administered to the affected mammal.

In another facet of the present invention, the compounds according to this invention show interesting properties, which may make them useful in the therapy of T-cell associated diseases, for suppression of the immune system, for treating restenosis and/or, if appropriate, for modulating angiogenesis.

The invention further includes a method for treating hyperproliferative diseases and/or disorders responsive to the induction of apoptosis, particularly those diseases, disorders, conditions or illnesses mentioned above, in mammals, including humans, suffering therefrom comprising administering to said mammals in need thereof a pharmacologically active and therapeutically effective and tolerable amount of one or more of the compounds according to this invention.

The present invention further includes a therapeutic method useful to modulate apoptosis in vivo or aberrant cell growth in benign or malignant neoplastic diseases, such as e.g. cancer, comprising administering to a subject in need of such therapy a therapeutically active and pharmacologically effective and tolerable amount of one or more of the compounds according to this invention which function by arresting aberrant cell growth and/or inducing apoptosis.

The present invention further relates to the use of the compounds according to this invention for the production of pharmaceutical compositions which are employed for the treatment, prophylaxis and/or amelioration of the illnesses mentioned.

The present invention further relates to the use of the compounds according to this invention for the production of pharmaceutical compositions which can be used in the treatment, prevention or amelioration of hyperproliferative diseases of benign or malignant behaviour and/or disorders responsive to the induction of apoptosis in a mammal, such as e.g. cancer.

The present invention further relates to the use of the compounds according to this invention for the production of pharmaceutical compositions which can be used use in the treatment, prevention or amelioration of disorders responsive to arresting of aberrant cell growth and/or induction of apoptosis.

The present invention further relates to pharmaceutical compositions comprising one or more of the compounds according to this invention and a pharmaceutically acceptable carrier or diluent.

The present invention further relates to combinations comprising one or more of the compounds according to this invention and pharmaceutically acceptable auxiliaries, excipients or vehicles, e.g. for use in the treatment, prevention or amelioration of hyperproliferative diseases of benign or malignant behaviour and/or disorders responsive to the induction of apoptosis in a mammal, such as e.g. cancer.

The present invention further relates to a composition consisting essentially of a therapeutically effective and tolerable amount of one or more pyrrolodihydroisoquinoline compounds according to this invention together with the usual pharmaceutically acceptable vehicles, diluents and/or excipients for use in therapy, e.g. for treating, preventing or ameliorating hyperproliferative diseases, such as e.g. cancer, and/or disorders responsive to induction of apoptosis.

The present invention further relates to compounds according to this invention for use in therapy, such as, for example, in the treatment, prevention or amelioration of those diseases mentioned herein, such as e.g. hyperproliferative diseases of benign or malignant behaviour and/or disorders responsive to the induction of apoptosis, particularly cancer.

The present invention further relates to compounds according to this invention having anti-proliferative and/or apoptosis inducing activity.

The invention further relates to the use of a pharmaceutical composition comprising one or more of the compounds according to this invention as sole active ingredient(s) and a pharmaceutically acceptable carrier or diluent in the manufacture of pharmaceutical products for the treatment and/or prophylaxis of the illnesses mentioned above.

The pharmaceutical compositions according to this invention are prepared by processes which are known per se and familiar to the person skilled in the art. As pharmaceutical compositions, the compounds of the invention (=active compounds) are either employed as such, or preferably in combination with suitable pharmaceutical auxiliaries and/or excipients, e.g. in the form of tablets, coated tablets, capsules, caplets, suppositories, patches (e.g. as TTS), emulsions, suspensions, gels or solutions, the active compound content advantageously being between 0.1 and 95% and where, by the appropriate choice of the auxiliaries and/or excipients, a pharmaceutical administration form (e.g. a delayed release form or an enteric form) exactly suited to the active compound and/or to the desired onset of action can be achieved.

The person skilled in the art is familiar with auxiliaries, vehicles, excipients, diluents, carriers or adjuvants which are suitable for the desired pharmaceutical formulations, preparations or compositions on account of his/her expert knowledge. In addition to solvents, gel formers, ointment bases and other active compound excipients, for example antioxidants, dispersants, emulsifiers, preservatives, solubilizers, colorants, complexing agents or permeation promoters, can be used.

Depending upon the particular disease, to be treated or prevented, additional therapeutic active agents, which are normally administered to treat or prevent that disease, may optionally be coadministered with the compounds according to this invention. As used herein, additional therapeutic agents that are normally administered to treat or prevent a particular disease are known as appropriate

for the disease being treated. For example, the compounds according to this invention may be combined with one or more known anti-cancer chemotherapeutic agents and/or with other target specific anti-cancer agents as described below.

Examples of known chemotherapeutic anti-cancer agents frequently used for combination therapy include, but not are limited to (i) alkylating/carbamylating agents such as Cyclophosphamid (Endoxan®), ifosfamid (Holoxan®), Thiotepa (Thiotehpa Lederle®), Melphalan (Alkeran®), or chloroethylnitrosourea (BCNU); (ii) platinum derivatives like cis-platin (Platinex® BMS), oxaliplafin or carboplatin (Cabroplat® BMS); (iii) antimitotic agents/tubulin inhibitors such as vinca alkaloids (vincristine, vinblastine, vinorelbine), taxanes such as Taxol (Paclitaxel®), Taxotere (Docetaxel®) and analogs as well as new formulations and conjugates thereof; (iv) topoisomerase inhibitors such as anthracyclines such as Doxorubicin (Adriblastin®), epipodophyllotoxines (such as Etoposide (Etopophos®) and camptothecin analogs such as Topotecan (Hycamtin®); (v) pyrimidine antagonists such as 5-fluorouracil (5-FU), Capecitabine (Xeloda®), Arabinosylcytosine/Cytarabin (Alexan®) or Gemcitabine (Gemzar®); (vi) purin antagonists such as 6-mercaptopurine (Puri-Nethol®), 6-thioguanine or fludarabine (Fludara®) and finally (vii) folic acid antagonists such as methotrexate (Farmitrexat®).

Examples of target specific anti-cancer drug classes used in experimental or standard cancer therapy include but are not limited to (i) kinase inhibitors such as e.g. Glivec (Imatinib®), ZD-1839/Iressa (Gefitinib®) or OSI-774/Tarceva (Erlotinib®); (ii) proteasome inhibitors such as PS-341 (Velcade®); (iii) histone deacetylase inhibitors like SAHA, MS275, CI-994, Depsipeptide/FK228, LAQ-824 and butyrates; (iv) heat shock protein inhibitors like 17-allylaminogeldanamycin (17-AAG); (v) vascular targeting agents (VAT) like combretastatin A4 phosphate and anti-angiogenic drugs in general; (v) monoclonal antibodies such as Herceptin (Trastuzumab®) or MabThera/Rituxan (Rituximab®) and conjugates of monoclonal antibodies and antibody fragments; (vi) oligonucleotide based therapeutics like G-3139/Genasense (Oblimersen®); (vii) protease inhibitors (viii) hormonal therapeutics such as anti-estrogens (e.g. Tamoxifen), anti-androgens (e.g. Flutamide or Casodex), LHRH analogs (e.g. Leuprolide, Goserelin or Triptorelin) and aromatase inhibitors.

Other known anti-cancer agents which can be used for combination therapy include bleomycin, retinoids such as all-trans retinoic acid (ATRA), DNA methyltransferase inhibitors such as the 2-deoxycytidine derivative Decitabine (Docagen®), alanosine, cytokines such as interleukin-2 or interferons such as interferon α2 or interferon-γ.

As exemplary chemotherapeutic/anti-cancer agents for use in the combination therapy according to the present invention the following drugs may be mentioned, without being restricted thereto, 5 FU, actinomycin D, ABARELIX, ABCIXIMAB, ACLARUBICIN, ADAPALENE, ALEMTUZUMAB, ALTRETAMINE, AMINOGLUTETHIMIDE, AMIPRILOSE, AMRUBICIN, ANASTROZOLE, ANCITABINE, ARTEMISININ, AZATHIOPRINE, BASILIXIMAB, BENDAMUSTINE, BICALUTAMIDE, BLEOMYCIN, BROXURIDINE, BUSULFAN, CAPECITABINE, CARBOPLATIN, CARBOQUONE, CARMUSTINE, CETRORELIX, CHLORAMBUCIL, CHLORMETHINE, CISPLATIN, CLADRIBINE, CLOMIFENE, CYCLOPHOSPHAMIDE, DACARBAZINE, DACLIZUMAB, DACTINOMYCIN, DAUNORUBICIN, DESLORELIN, DEXRAZOXANE, DOCETAXEL, DOXIFLURIDINE, DOXORUBICIN, DROLOXIFENE, DROSTANOLONE, EDELFOSINE, EFLORNITHINE, EMITEFUR, EPIRUBICIN, EPITIOSTANOL, EPTAPLATIN, ERBITUX, ESTRAMUSTINE, ETOPOSIDE, EXEMESTANE, FADROZOLE, FINASTERIDE, FLOXURIDINE, FLUCYTOSINE, FLUDARABINE, FLUOROURACIL, FLUTAMIDE, FORMESTANE, FOSCARNET, FOSFESTROL, FOTEMUSTINE, FULVESTRANT, GEFITINIB, GEMCITABINE, GLIVEC, GOSERELIN, GUSPERIMUS, HERCEPTIN, IDARUBICIN, IDOXURIDINE, IFOSFAMIDE, IMATINIB, IMPROSULFAN, INFLIXIMAB, IRINOTECAN, LANREOTIDE, LETROZOLE, LEUPRORELIN, LOBAPLATIN, LOMUSTINE, MELPHALAN, MERCAPTOPURINE, METHOTREXATE, METUREDEPA, MIBOPLATIN, MIFEPRISTONE, MILTEFOSINE, MIRIMOSTIM, MITOGUAZONE, MITOLACTOL, MITOMYCIN, MITOXANTRONE, MIZORIBINE, MOTEXAFIN, NARTOGRASTIM, NEBAZUMAB, NEDAPLATIN, NILUTAMIDE, NIMUSTINE, OCTREOTIDE, ORMELOXIFENE, OXALIPLATIN, PACLITAXEL, PALIVIZUMAB, PEGASPARGASE, PEGFILGRASTIM, PENTETREOTIDE, PENTOSTATIN, PERFOSFAMIDE, PIPOSULFAN, PIRARUBICIN, PLICAMYCIN, PREDNIMUSTINE, PROCARBAZINE, PROPAGERMANIUM, PROSPIDIUM CHLORIDE, RALTITREXED, RANIMUSTINE, RANPIRNASE, RASBURICASE, RAZOXANE, RITUXIMAB, RIFAMPICIN, RITROSULFAN, ROMURTIDE, RUBOXISTAURIN, SARGRAMOSTIM, SATRAPLATIN, SIROLIMUS, SOBUZOXANE, SPIROMUSTINE, STREPTOZOCIN, TAMOXIFEN, TASONERMIN, TEGAFUR, TEMOPORFIN, TEMOZOLOMIDE, TENIPOSIDE, TESTOLACTONE, THIOTEPA, THYMALFASIN, TIAMIPRINE, TOPOTECAN, TOREMIFENE, TRASTUZUMAB, TREOSULFAN, TRIAZIQUONE, TRIMETREXATE, TRIPTORELIN, TROFOSFAMIDE, UREDEPA, VALRUBICIN, VERTEPORFIN, VINBLASTINE, VINCRISTINE, VINDESINE, VINORELBINE and VOROZOLE.

The person skilled in the art is aware on the base of his/her expert knowledge of the total daily dosage(s) of the additional therapeutic agent(s) coadministered. Said total daily dosage(s) can vary within a wide range.

In a facet of the present invention, examples of known anti-cancer agents include, but are not limited to, Gleevec, Herceptin, Rituxan, Adriamycin, Vincristine, Cyclophosphamide and ifosfamide, 5-Fluorouracil, Topotecan, Doxorubicin, Paclitaxel Craxol), interferons, and Platinum derivatives like Cisplatin or Oxaliplatin.

In practicing the present invention, the compounds according to this invention may be administered in combination therapy separately, sequentially, simultaneously or chronologically staggered (such as e.g. as combined unit dosage forms, as separate unit dosage forms, as adjacent discrete unit dosage forms, as fixed or non-fixed combinations, as kit-of-parts or as admixtures) with one or more known anti-cancer agents or target specific anti-cancer agents, such as e.g. those mentioned above.

In this context, the present invention further relates to a combination comprising

a first active ingredient, which is at least one pyrrolodihydroisoquinoline compound according to this invention, and

a second active ingredient, which is at least one anti-cancer agent or target specific anti-cancer agent, such as e.g. one or more of those mentioned herein above,

for separate, sequential, simultaneous or chronologically staggered use in therapy, such as e.g. to treat, prevent or ameliorate hyperproliferative diseases of benign or malignant behaviour and/or disorders responsive to the induction of apoptosis in a mammal, such as e.g. those diseases mentioned herein, for example cancer.

The term ‘combination’ according to this invention may be present as a fixed combination, a non-fixed combination or a kit-of-parts.

A “fixed combination” is defined as a combination wherein the said first active ingredient and the said second active ingredient are present together in one unit dosage or in a single entity. One example of a “fixed combination” is a pharmaceutical composition wherein the said first active ingredient and the said second active ingredient are present in admixture for simultaneous administration, such as in a formulation. Another example of a “fixed combination” is a pharmaceutical combination wherein the said first active ingredient and the said second active ingredient are present in one unit without being in admixture.

A “kit-of-parts” is defined as a combination wherein the said first active ingredient and the said second active ingredient are present in more than one unit. One example of a “kit-of-parts” is a combination wherein the said first active ingredient and the said second active ingredient are present separately. The components of the kit-of-parts may be administered separately, sequentially, simultaneously or chronologically staggered.

The present invention further relates to a pharmaceutical composition comprising

a first active ingredient, which is at least one pyrrolodihydroisoquinoline compound according to this invention, and

a second active ingredient, which is at least one anti-cancer agent or target specific anti-cancer agent, such as e.g. one or more of those mentioned herein above, and, optionally,

a pharmaceutically acceptable carrier or diluent,

for separate, sequential, simultaneous or chronologically staggered use in therapy.

The present invention further relates to a kit-of-parts comprising a preparation of a first active ingredient, which is a pyrrolodihydroisoquinoline compound according to this invention, and a pharmaceutically acceptable carrier or diluent; a preparation of a second active ingredient, which is an art-known anti-cancer and/or target specific anti-cancer agent, such as one of those mentioned above, and a pharmaceutically acceptable carrier or diluent; for simultaneous, sequential, separate or chronologically staggered use in therapy. Optionally, said kit comprises instructions for its use in therapy, e.g. to treat hyperproliferative diseases and/or disorders responsive to the induction of apoptosis, such as e.g. cancer.

The present invention further relates to a combined preparation comprising at least one compound according to the present invention and at least one anti-cancer and/or target specific anticancer agent for simultaneous, sequential or separate administration.

The present invention further relates to pharmaceutical combinations or compositions according to this invention having anti-proliferative and/or apoptosis inducing activity.

In addition, the present invention further relates to a method for treating hyperproliferative diseases and/or disorders responsive to the induction of apoptosis, such as e.g. cancer, in a patient comprising administering a combination, composition, formulation, preparation or kit as described herein to said patient in need thereof.

In addition, the present invention further relates to a method for treating hyperproliferative diseases of benign or malignant behaviour and/or disorders responsive to the induction of apoptosis, such as e.g. cancer, in a patient comprising administering in combination therapy separately, simultaneously, sequentially or chronologically staggered a pharmaceutically active and therapeutically effective and tolerable amount of a pharmaceutical composition, which comprises a pyrrolodihydroisoquinoline compound according to this invention and a pharmaceutically acceptable carrier or diluent, and a pharmaceutically active and therapeutically effective and tolerable amount of one or more anti-cancer and/or target specific anti-cancer agents, such as e.g. one or more of those mentioned herein, to said patient in need thereof.

In addition, the present invention further relates to the use of a composition, combination, formulation, preparation or kit in the manufacture of a pharmaceutical product, such as e.g. a commercial package or a medicament, for treating, preventing or ameliorating hyperproliferative diseases, such as e.g. cancer, and/or disorders responsive to the induction of apoptosis, particularly those diseases mentioned herein.

The present invention further relates to a commercial package comprising one or more compounds of the present invention together with instructions for simultaneous, sequential or separate use with one or more chemotherapeutic and/or target specific anti-cancer agents, such as e.g. any of those mentioned herein.

The present invention further relates to a commercial package consisting essentially of one or more compounds of the present invention as sole active ingredient together with instructions for simultaneous, sequential or separate use with one or more chemotherapeutic and/or target specific anti-cancer agents, such as e.g. any of those mentioned herein.

The present invention further relates to a commercial package comprising one or more chemotherapeutic and/or target specific anti-cancer agents, such as e.g. any of those mentioned herein, together with instructions for simultaneous, sequential or separate use with one or more pyrrolodihydroisoquinoline compounds according to the present invention.

The compositions, combinations, preparations, formulations, kits or packages mentioned in the context of the combination therapy according to this invention may also include more than one of the compounds according to this invention and/or more than one of the art-known anti-cancer and/or target specific anticancer agents mentioned.

In addition, compounds according to the present invention can be used in the pre- or post-surgical treatment of cancer.

In further addition, compounds of the present invention can be used in combination with radiation therapy.

A combination according to this invention can refer to a composition comprising both the compounds according to this invention and the other active anti-cancer agent in a fixed combination (fixed unit dosage form), or a medicament pack comprising the two active ingredients as discrete separate dosage forms (non-fixed combination). In case of a medicament pack comprising the two active ingredients, the active ingredients are preferably packed into blister cards which are suited for improving compliance.

Each blister card preferably contains the medicaments to be taken on one day of treatment. If the medicaments are to be taken at different times of day, the medicaments can be disposed in different sections on the blister card according to the different ranges of times of day at which the medicaments are to be taken (for example morning and evening or morning, midday and evening). The blister cavities for the medicaments to be taken together at a particular time of day are accommodated in the respective range of times of day. The various times of day are, of course, also put on the blister in a dearly visible way. It is also possible, of course, for example to indicate a period in which the medicaments are to be taken, for example stating the times.

The daily sections may represent one line of the blister card, and the times of day are then identified in chronological sequence in this column.

Medicaments which must be taken together at a particular time of day are placed together at the appropriate time on the blister card, preferably a narrow distance apart, allowing them to be pushed out of the blister easily, and having the effect that removal of the dosage form from the blister is not forgotten.

The administration of the pharmaceutical compositions or combinations according to the invention may be performed in any of the generally accepted modes of administration available in the art illustrative examples of suitable modes of administration include intravenous, oral, nasal, parenteral, topical, transdermal and rectal delivery. Oral and intravenous delivery are preferred.

For the treatment of dermatoses, the compounds of the invention are in particular administered in the form of those pharmaceutical compositions which are suitable for topical application. For the production of the pharmaceutical compositions, the compounds of the invention (=active compounds) are preferably mixed with suitable pharmaceutical auxiliaries and further processed to give suitable pharmaceutical formulations. Suitable pharmaceutical formulations are, for example, powders, emulsions, suspensions, sprays, oils, ointments, fatty ointments, creams, pastes, gels or solutions.

The pharmaceutical compositions according to the invention are prepared by processes known per se. The dosage of the active compounds is carried out in the order of magnitude customary for inhibitors of cellular proliferation or apoptosis inducers. Topical application forms (such as ointments) for the treatment of dermatoses thus contain the active compounds in a concentration of, for example, 0.1-99%. The customary dose in the case of systemic therapy (p.o.) is between 0.3 and 30 mg/kg per day, (i.v.) is between 0.3 and 30 mg/kg/h.

Biological Investigations

The anti-proliferative/cytotoxic activity of the compounds described herein, can be tested on NCl-H460 non-small cell lung cancer cells using the Alamar Blue cell viability assay (described in O'Brien et al. Eur J Biochem 267, 5421-5426, 2000). The compounds are dissolved as 20 mM solutions in dimethylsulfoxide (DMSO) and subsequently diluted in semi-logarithmic steps. DMSO dilutions are further diluted 1:100 into Dulbecco's modified Eagle's medium (DMEM) containing 10% fetal calf serum to a final concentration twice as much as the final concentration in the test. NCl-460 cells are seeded into 96 well flat bottom plates at a density of 4000 cells per well in a volume of 50 μl per well. 24 hours after seeding the 50 μl each of the compound dilutions in DMEM medium are added into each well of the 96 Well plate. Each compound dilution is tested as quadruplicates. Wells containing untreated control cells are filled with 50 μl DMEM medium containing 1% DMSO. The cells are then incubated with the substances for 72 hours at 37° C. In a humified atmosphere containing 5% carbon dioxide. To determine the viability of the cells, 10 μl of an Alamar Blue solution (Biosource) are added and the fluorescence is measured at an extinction of 544 nm and an emission of 590 nm. For the calculation of cell viability the emission value from untreated cells is set to 100% viability and the emission rates of treated cells are set in relation to the values of untreated cells. Viabilities are expressed as % values.

The corresponding IC₅₀ values of the compounds for anti-proliferative/cytotoxic activity are determined from the concentration-effect curves.

Representative advantageous IC₅₀ values for anti-proliferation/cytotoxicity determined for the compounds mentioned and numbered as Examples in the examples above follow from the following table A, in which the numbers of the compound correspond to the numbers of the examples.

TABLE A
Anti-proliferative/cytotoxic activity

| | −log IC₅₀ NCl- |
|---|---|
| Compounds | H460 (mol/l) |
| 1, 2, 4, 5, 9, | The inhibitory |
| 10, 16, 18, | values of these |
| 20, 21, 22, | listed Examples |
| 25, 28, 30, | lie in the range |
| 34 and 36 | from 5.5 to 6.4 |

The induction of apoptosis can be measured by using a Cell death detection ELISA (Roche Biochemicals, Mannheim, Germany). NCl-460 cells are seeded into 96 well flat bottom plates at a density of 10000 cells per well in a volume of 50 μl per well. 24 hours after seeding the 50 μl each of the compound dilutions in DMEM medium are added into each well of the 96 Well plate. Each compound dilution is tested at least as triplicates. Wells containing untreated control cells are filled with 50 μl DMEM medium containing 1% DMSO. The cells are then incubated with the substances for 24 hours at 37° C. in a humidified athmosphere containing 5% carbon dioxide. As a positive control for the induction of apoptosis, cells are treated with 50 μM Cisplatin (Gry Pharmaceuticals, Kirchzarten, Germany). Medium is then removed and the cells are lysed in 200 μl lysis buffer. After centrifugation as described by the manufacturer, 10 μl of cell lysate is processed as described in the protocol. The degree of apoptosis is calculated as follows: The absorbance at 405 nm obtained with lysates from cells treated with 50 μM cisplatin is set as 100 cpu (cisplatin units), while an absorbance at 405 nm of 0.0 was set as 0.0 cpu. The degree of apoptosis is expressed as cpu in relation to the value of 100 cpu reached with the lysates obtained from cells treated with 50 μM cisplatin.

1. A compound of formula I 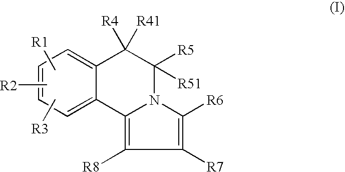
in which
R1 is halogen, nitro, amino, mono- or di-1-4C-alkylamino, 1-4C-alkyl, hydroxyl, 1-4C-alkoxy, 1-4C-alkoxy-2-4C-alkoxy, 3-7C-cycloalkoxy, 3-7C-cycloalkylmethoxy, or completely or predominantly fluorine-substituted 1-4C-alkoxy,
R2 is hydrogen, halogen or 1-4C-alkoxy, and
R3 is hydrogen or 1-4C-alkoxy, or
R2 and R3 bound to the benzo ring moiety in ortho-position to each other together form a 1-2C-alkylenedioxy bridge, or
R2 and R3 bound to the benzo ring moiety in ortho-position to each other together form a completely or predominantly fluorine-substituted 1-2C-alkylenedioxy bridge, or
R1 and R2 bound to the benzo ring moiety in ortho-position to each other together form a 1-2C-alkylenedioxy bridge and R3 is hydrogen, or
R1 and R2 bound to the benzo ring moiety in ortho-position to each other together form a completely or predominantly fluorine-substituted 1-2C-alkylenedioxy bridge and R3 is hydrogen,
R4 is hydrogen, fluorine, chlorine, 1-4C-alkyl, trifluoromethyl, cyclopropyl, cyano, 1-4C-alkoxycarbonyl or —CH₂—O—R411, in which
R411 is hydrogen, 1-4C-alkyl, 1-4C-alkoxy-2-4-alkyl or 1-4C-alkylcarbonyl,
R41 is hydrogen or 1-4C-alkyl,
R5 is hydrogen, and
R51 is hydrogen,
or
R4 is hydrogen, fluorine, chlorine or 1-4C-alkyl,
R41 is hydrogen or 1-4C-alkyl,
R5 is hydrogen, and
R51 is hydrogen,
R6 is 1-6C-alkyl, amino, formyl, or 1-4C-alkyl substituted by R61, in which
R61 is 1-4C-alkoxycarbonyl, carboxyl, 1-4C-alkoxy, hydroxyl, halogen or —N(R611)R612, in which
R611 is hydrogen, 1-4C-alkyl, 3-7C-cycloalkyl or 3-7C-cycloalkyl-1-4C-alkyl, and
R612 is hydrogen or 1-4C-alkyl, or
R611 and R612 together and with inclusion of the nitrogen atom to which they are bound form a radical Het1, in
which
Het1 is a 5- to 7-membered saturated heterocyclic ring radical comprising one nitrogen atom, to which R611 and R612 are bound, and, optionally, one further heteroatom selected from the group consisting of nitrogen, oxygen and sulfur, and optionally substituted by R613 on a ring nitrogen atom, in which
R613 is 1-4C-alkyl, 3-7C-cycloalkyl, 3-7C-cycloalkyl-1-4C-alkyl, hydroxy-2-4C-alkyl, 1-4C-alkoxy-2-4C-alkyl, amino-2-4C-alkyl, mono- or di-1-4C-alkylamino-2-4C-alkyl, formyl, pyridyl or pyrimidinyl,
R7 is phenyl, Het2, R71- and/or R72- and/or R73-substituted phenyl, R74- and/or R75-substituted Het2, naphthyl, or R76- and/or R77-substituted naphthyl, in which
Het2 is a monocyclic or fused bicyclic 5 to 10-membered heteroaryl radical comprising one to three heteroatoms, each of which is selected from the group consisting of nitrogen, oxygen and sulfur,
R71 is hydroxyl, halogen, nitro, cyano, trifluoromethyl, 1-4C-alkyl, 1-4C-alkoxy, amino, mono- or di-1-4C-alkylamino, 1-4C-alkylsulphonylamino, arylsulphonylamino, 1-4C-alkoxycarbonyl, carboxyl, 1-4C-alkylthio, aryloxy-2-4C-alkoxy, aryloxy-1-4C-alkyl, aryloxy, aryl-1-4C-alkoxy, aryl, 1-4C-alkoxy-2-4C-alkoxy, 1-4C-alkoxy-1-4C-alkyl, hydroxy-2-4C-alkoxy, amino-2-4C-alkoxy, mono- or di-1-4C-alkylamino-2-4C-alkoxy, or completely or predominantly fluorine-substituted 1-4C-alkoxy, in which
aryl is phenyl or R711-substituted phenyl, in which
R711 is halogen, 1-4C-alkyl, 1-4C-alkoxy, nitro or cyano,
R72 is halogen, 1-4C-alkyl, 1-4C-alkoxy or 1-4C-alkoxycarbonyl,
R73 is 1-4C-alkyl or 1-4C-alkoxy,
R74 is halogen, 1-4C-alkyl, trifluoromethyl, 1-4C-alkoxy, cyano, amino, mono- or di-1-4C-alkylamino, 1-4C-alkoxycarbonyl, morpholino, carboxyl, nitro, phenyl or phenyloxy,
R75 is 1-4C-alkyl or halogen,
R76 is halogen, hydroxyl, 1-4C-alkyl, 1-4C-alkoxy, carboxyl or 1-4C-alkoxycarbonyl,
R77 is 1-4C-alkyl or 1-4C-alkoxy,
R8 is phenyl, phenylcarbonyl, —C(O)—N(R82)R83 or —C(O)—OR9, in which
R82 is hydrogen, 1-4C-alkyl, 3-7C-cycloalkyl, 3-7C-cycloalkyl-1-4C-alkyl, phenyl or phenyl-1-4C-alkyl, and
R83 is hydrogen or 1-4C-alkyl, or
R82 and R83 together and with inclusion of the nitrogen atom, to which they are bound, form a heterocyclic ring radical selected from the group consisting of pyrrolidinyl, piperidinyl, morpholinyl and N-(1-4C-alkyl)-piperazinyl,
R9 is 1-4C-alkyl,
or a salt, stereoisomer, hydrate or hydrate of a salt thereof;
under the proviso that this subgroup of compounds of formula I, wherein the combination of all of the following restrictions a.) to c.) apply, is thereof disclaimed:
a.) the substitution pattern of the left R1- and/or R2- and/or R3-substituted benzo ring of the dihydroisoquinoline moiety of the pyrrolodihydroisoquinoline scaffold shown in formula I is as follows: 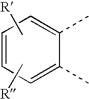
in which
R′ and R″ can be bonded at any possible position of the benzo ring, and
R′ is hydroxyl, 1-4C-alkoxy or trifluoromethoxy,
R″ is hydrogen or 1-4C-alkoxy,
or R′ and R″ bound to the benzo ring moiety in ortho-position to each other together form a 1-2C-alkylenedioxy bridge,
and
b.) R4 is hydrogen, and
R41 is hydrogen, and
R5 is hydrogen, and
R51 is hydrogen,
and
c.) R8 is —C(O)—OR9, in which
R9 is 1-4C-alkyl.

2. A compound of formula I according to claim 1, in which
R1 is halogen, nitro, amino, 1-4C-alkyl, 1-4C-alkoxy, 1-4C-alkoxy-2-4C-alkoxy, or completely or predominantly fluorine-substituted 1-4C-alkoxy,
R2 is 1-4C-alkoxy,
R3 is hydrogen,
and none of R1, R2 and R3 is bound to the 10-position of the pyrrolo[2,1-a]-isoquinoline ring,
R4 is hydrogen or 1-4C-alkyl,
R41 is hydrogen or 1-4C-alkyl,
R5 is hydrogen,
R51 is hydrogen,
R6 is 1-6C-alkyl, formyl, or 1-4C-alkyl substituted by R61, in which
R61 is 1-4C-alkoxycarbonyl,
R7 is phenyl, Het2, R71- and/or R72- and/or R73-substituted phenyl, R74- and/or R75-substituted Het2, or naphthyl, in which
Het2 is a heteroaryl radical selected from the group consisting of furanyl, thiophenyl, pyrrolyl, pyridinyl, quinolyl, indolyl, benzothiophenyl and benzofuranyl,
R71 is hydroxyl, 1-4C-alkoxy, amino or mono- or di-1-4C-alkylamino,
R72 is 1-4C-alkyl or 1-4C-alkoxy,
R73 is 1-4C-alkyl or 1-4C-alkoxy,
R74 is 1-4C-alkyl, trifluoromethyl, 1-4C-alkoxy, 1-4C-alkoxycarbonyl, nitro, phenyl or phenyloxy,
R75 is 1-4C-alkyl,
R8 is phenyl, phenylcarbonyl, or —C(O)—N(R82)R83, in which
R82 is hydrogen, 1-4C-alkyl, 3-7C-cycloalkyl or phenyl, and R83 is hydrogen or 1-4C-alkyl, or R82 and R83 together and with inclusion of the nitrogen atom, to which they are bound, form a heterocyclic ring radical selected from the group consisting of pyrrolidinyl and piperidinyl,
or a salt, stereoisomer, hydrate or hydrate of a salt thereof.

3. A compound of formula I according to claim 1, in which
R1 is halogen, nitro, amino, 1-4C-alkyl, 1-4C-alkoxy-2-4C-alkoxy, or completely or predominantly fluorine-substituted 1-4C-alkoxy,
with the provisio that R1 is not trifluoromethoxy,
R2 is 1-4C-alkoxy, and
R3 is hydrogen, or
R1 and R2 bound to the benzo ring moiety in ortho-position to each other together form a completely or predominantly fluorine-substituted 1-2C-alkylenedioxy bridge and R3 is hydrogen,
and none of R1, R2 and R3 is bound to the 10-position of the pyrrolo[2,1-a]-isoquinoline ring,
R4 is hydrogen or 1-4C-alkyl,
R41 is hydrogen or 1-4C-alkyl,
R5 is hydrogen,
R51 is hydrogen,
R6 is 1-6C-alkyl, formyl, or 1-4C-alkyl substituted by R61, in which
R61 is 1-4C-alkoxycarbonyl,
R7 is phenyl, Het2, R71- and/or R72- and/or R73-substituted phenyl, R74- and/or R75-substituted Het2, or naphthyl, in which
Het2 is a heteroaryl radical selected from the group consisting of furanyl, thiophenyl, pyrrolyl, pyridinyl, quinolyl, indolyl, benzothiophenyl and benzofuranyl,
R71 is hydroxyl, 1-4C-alkoxy, amino or mono- or di-1-4C-alkylamino,
R72 is 1-4C-alkyl or 1-4C-alkoxy,
R73 is 1-4C-alkyl or 1-4C-alkoxy,
R74 is 1-4C-alkyl, trifluoromethyl, 1-4C-alkoxy, 1-4C-alkoxycarbonyl, nitro, phenyl or phenyloxy,
R75 is 1-4C-alkyl,
R8 is phenyl, phenylcarbonyl, —C(O)—N(R82)R83 or —C(O)—OR9, in which
R82 is hydrogen, 1-4C-alkyl, 3-7C-cycloalkyl or phenyl, and
R83 is hydrogen or 1-4C-alkyl, or
R82 and R83 together and with inclusion of the nitrogen atom, to which they are bound, form a heterocyclic ring radical selected from the group consisting of pyrrolidinyl and piperidinyl,
R9 is 1-4C-alkyl,
or a salt, stereoisomer, hydrate or hydrate of a salt thereof.

4. A compound of formula I according to claim 1, in which
R1 is halogen, nitro, amino, 1-4C-alkyl, 1-4C-alkoxy, 1-4C-alkoxy-2-4C-alkoxy, or completely or predominantly fluorine-substituted 1-4C-alkoxy,
R2 is 1-4C-alkoxy,
R3 is 1-4C-alkoxy,
and none of R1, R2 and R3 is bound to the 10-position of the pyrrolo[2,1-a]-isoquinoline ring,
R4 is hydrogen or 1-4C-alkyl,
R41 is hydrogen or 1-4C-alkyl,
R5 is hydrogen,
R51 is hydrogen,
R6 is 1-6C-alkyl, formyl, or 1-4C-alkyl substituted by R61, in which
R61 is 1-4C-alkoxycarbonyl,
R7 is phenyl, Het2, R71- and/or R72- and/or R73-substituted phenyl, R74- and/or R75-substituted Het2, or naphthyl, in which
Het2 is a heteroaryl radical selected from the group consisting of furanyl, thiophenyl, pyrrolyl, pyridinyl, quinolyl, indolyl, benzothiophenyl and benzofuranyl,
R71 is hydroxyl, 1-4C-alkoxy, amino or mono- or di-1-4C-alkylamino,
R72 is 1-4C-alkyl or 1-4C-alkoxy,
R73 is 1-4C-alkyl or 1-4C-alkoxy,
R74 is 1-4C-alkyl, trifluoromethyl, 1-4C-alkoxy, 1-4C-alkoxycarbonyl, nitro, phenyl or phenyloxy,
R75 is 1-4C-alkyl,
R8 is phenyl, phenylcarbonyl, —C(O)—N(R82)R83 or —C(O)—OR9, in which
R82 is hydrogen, 1-4C-alkyl, 3-7C-cycloalkyl or phenyl, and
R83 is hydrogen or 1-4C-alkyl, or
R82 and R83 together and with inclusion of the nitrogen atom, to which they are bound, form a heterocyclic ring radical selected from the group consisting of pyrrolidinyl and piperidinyl,
R9 is 1-4C-alkyl,
or a salt, stereoisomer, hydrate or hydrate of a salt thereof.

5. A compound of formula I according to claim 1, in which
R1 is halogen, nitro, amino, 1-4C-alkyl, 1-4C-alkoxy, 1-4C-alkoxy-2-4C-alkoxy, or completely or predominantly fluorine-substituted 1-4C-alkoxy,
R2 is 1-4C-alkoxy,
R3 is hydrogen,
and none of R1, R2 and R3 is bound to the 10-position of the pyrrolo[2,1-a]-isoquinoline ring,
R4 is 1-4C-alkyl,
R41 is hydrogen or 1-4C-alkyl,
R5 is hydrogen,
R51 is hydrogen,
R6 is 1-6C-alkyl, formyl, or 1-4C-alkyl substituted by R61, in which
R61 is 1-4C-alkoxycarbonyl,
R7 is phenyl, Het2, R71- and/or R72- and/or R73-substituted phenyl, R74- and/or R75-substituted Het2, or naphthyl, in which
Het2 is a heteroaryl radical selected from the group consisting of furanyl, thiophenyl, pyrrolyl, pyridinyl, quinolyl, indolyl, benzothiophenyl and benzofuranyl,
R71 is hydroxyl, 1-4C-alkoxy, amino or mono- or di-1-4C-alkylamino,
R72 is 1-4C-alkyl or 1-4C-alkoxy,
R73 is 1-4C-alkyl or 1-4C-alkoxy,
R74 is 1-4C-alkyl, trifluoromethyl, 1-4C-alkoxy, 1-4C-alkoxycarbonyl, nitro, phenyl or phenyloxy,
R75 is 1-4C-alkyl,
R8 is phenyl, phenylcarbonyl, —C(O)—N(R82)R83 or —C(O)—OR9, in which
R82 is hydrogen, 1-4C-alkyl, 3-7C-cycloalkyl or phenyl, and
R83 is hydrogen or 1-4C-alkyl, or
R82 and R83 together and with inclusion of the nitrogen atom, to which they are bound, form a heterocyclic ring radical selected from the group consisting of pyrrolidinyl and piperidinyl,
R9 is 1-4C-alkyl,
or a salt, stereoisomer, hydrate or hydrate of a salt thereof.

6. A compound of formula I according to claim 1,
in which, in a first embodiment,
R1 is halogen, nitro, amino, mono- or di-1-4C-alkylamino, 1-4C-alkyl, 1-4C-alkoxy-2-4C-alkoxy, 3-7C-cycloalkoxy, 3-7C-cycloalkylmethoxy, or completely or predominantly fluorine-substituted 1-4C-alkoxy,
R2 is 1-4C-alkoxy,
R3 is hydrogen,
and none of R1 and R2 is bound to the 10-position of the pyrrolo[2,1-a]-isoquinoline ring,
and
R4 is hydrogen,
R41 is hydrogen,
R5 is hydrogen, and
R51 is hydrogen;
or in which, in a second embodiment,
R1 is 1-4C-alkoxy,
R2 is 1-4C-alkoxy,
R3 is hydrogen,
and none of R1 and R2 is bound to the 10-position of the pyrrolo[2,1-a]isoquinoline ring,
and
R4 is 1-4C-alkyl,
R41 is hydrogen or 1-4C-alkyl,
R5 is hydrogen, and
R51 is hydrogen;
R6 is 1-6C-alkyl, or 1-4C-alkyl substituted by R61, in
which
R61 is 1-4C-alkoxycarbonyl;
R7 is Het2,4-hydroxy-3,5-dimethylphenyl, 3-dimethylamino-phenyl, or naphthyl, in which
Het2 is a monocyclic or fused bicyclic 5- to 10-membered heteroaryl radical comprising one to three heteroatoms, each of which is selected from the group consisting of nitrogen, oxygen and sulfur;
R8 is phenyl, phenylcarbonyl, —C(O)—N(R82)R83 or —C(O)—OR9, in which
R82 is hydrogen, 1-4C-alkyl, 3-7C-cycloalkyl or phenyl, and
R83 is hydrogen or 1-4C-alkyl, or
R82 and R83 together and with inclusion of the nitrogen atom, to which they are bound, form a heterocyclic ring radical selected from the group consisting of pyrrolidinyl and piperidinyl,
R9 is 1-4C-alkyl;
or a salt, stereoisomer, hydrate or hydrate of a salt thereof.

7. A compound of formula I according to claim 1,
in which, in a first embodiment,
R1 is halogen, 1-4C-alkoxy-2-4C-alkoxy, or completely or predominantly fluorine-substituted 1-4C-alkoxy,
R2 is 1-4C-alkoxy,
R3 is hydrogen,
and none of R1 and R2 is bound to the 7- or 10-position of the pyrrolo[2,1-a]-isoquinoline ring,
and
R4 is hydrogen,
R41 is hydrogen,
R5 is hydrogen, and
R51 is hydrogen;
or in which, in a second embodiment,
R1 is 1-4C-alkoxy,
R2 is 1-4C-alkoxy,
R3 is hydrogen,
and none of R1 and R2 is bound to the 7- or 10-position of the pyrrolo[2,1-a]-isoquinoline ring,
and
R4 is 1-4C-alkyl,
R41 is hydrogen or 1-4C-alkyl,
R5 is hydrogen, and
R51 is hydrogen;
R6 is 1-4C-alkyl, or 1-4C-alkyl substituted by R61, in
which
R61 is 1-4C-alkoxycarbonyl;
R7 is Het2,4-hydroxy-3,5-dimethylphenyl, 3-dimethylamino-phenyl, or naphthyl, in which
Het2 is a monocyclic or fused bicyclic 5- to 10-membered heteroaryl radical comprising one to three heteroatoms, each of which is selected from the group consisting of nitrogen, oxygen and sulfur,
R8 is —C(O)—OR9, in which
R9 is 1-4C-alkyl;
or a salt, stereoisomer, hydrate or hydrate of a salt thereof.

8. A compound of formula I according to claim 1,
in which, in a first embodiment,
either
R1 is halogen, 1-4C-alkyl, 1-4C-alkoxy, 1-4C-alkoxy-2-4C-alkoxy, or completely or predominantly fluorine-substituted 1-4C-alkoxy,
R2 is 1-4C-alkoxy, and
R3 is 1-4C-alkoxy,
or
R1 is 1-4C-alkoxy, 1-4C-alkoxy-2-4C-alkoxy, or completely or predominantly fluorine-substituted 1-4C-alkoxy,
R2 is halogen, and
R3 is 1-4C-alkoxy,
and none of R1, R2 and R3 is bound to the 10-position of the pyrrolo[2,1-a]-isoquinoline ring;
or
either
R1 is halogen, 1-4C-alkyl, 1-4C-alkoxy, 1-4C-alkoxy-2-4C-alkoxy, or completely or predominantly fluorine-substituted 1-4C-alkoxy,
R2 is 1-4C-alkoxy, and
R3 is hydrogen,
or
R1 is 1-4C-alkoxy, 1-4C-alkoxy-2-4C-alkoxy, or completely or predominantly fluorine-substituted 1-4C-alkoxy,
R2 is halogen, and
R3 is hydrogen,
and none of R1 and R2 is bound to the 7- or 10-position of the pyrrolo[2,1-a]-isoquinoline ring;
and
R4 is 1-4C-alkyl,
R41 is hydrogen or 1-4C-alkyl,
R5 is hydrogen, and
R51 is hydrogen;
or in which, in a second embodiment,
R1 is 1-4C-alkoxy,
R2 is 1-4C-alkoxy,
R3 is hydrogen,
and none of R1 and R2 is bound to the 7- or 10-position of the pyrrolo[2,1-a]-isoquinoline ring,
and
R4 is 1-4C-alkyl,
R41 is hydrogen or 1-4C-alkyl,
R5 is hydrogen,
R51 is hydrogen;
R6 is 1-6C-alkyl, or 1-4C-alkyl substituted by R61, in which
R61 is 1-4C-alkoxycarbonyl,
R7 is phenyl, Het2, R71- and/or R72- and/or R73-substituted phenyl, R74- and/or R75-substituted Het2, or naphthyl, in which
Het2 is a heteroaryl radical selected from the group consisting of furanyl, thiophenyl, pyrrolyl, pyridinyl, quinolyl, indolyl, benzothiophenyl and benzofuranyl,
R71 is hydroxyl, 1-4C-alkoxy, amino or mono- or di-1-4C-alkylamino,
R72 is 1-4C-alkyl or 1-4C-alkoxy,
R73 is 1-4C-alkyl or 1-4C-alkoxy,
R74 is 1-4C-alkyl, trifluoromethyl, 1-4C-alkoxy, 1-4C-alkoxycarbonyl, nitro, phenyl or phenyloxy,
R75 is 1-4C-alkyl,
R8 is phenyl, phenylcarbonyl, —C(O)—N(R82)R83 or —C(O)—OR9, in which
R82 is hydrogen, 1-4C-alkyl, 3-7C-cycloalkyl or phenyl, and
R83 is hydrogen or 1-4C-alkyl, or
R82 and R83 together and with inclusion of the nitrogen atom, to which they are bound, form a heterocyclic ring radical selected from the group consisting of pyrrolidinyl and piperidinyl,
R9 is 1-4C-alkyl,
or a salt, stereoisomer, hydrate or hydrate of a salt thereof.

9. A compound of formula I according to claim 1,
in which
R1 is halogen, 1-4C-alkoxy, 1-4C-alkoxy-2-4C-alkoxy, or completely or predominantly fluorine-substituted 1-4C-alkoxy,
R2 is 1-4C-alkoxy,
R3 is 1-4C-alkoxy,
and none of R1, R2 and R3 is bound to the 10-position of the pyrrolo[2,1-a]-isoquinoline ring,
and
R4 is hydrogen, or 1-4C-alkyl
R41 is hydrogen, or 1-4C-alkyl
R5 is hydrogen, and
R51 is hydrogen;
or
R1 is halogen, 1-4C-alkoxy-2-4C-alkoxy, or completely or predominantly fluorine-substituted 1-4C-alkoxy,
R2 is 1-4C-alkoxy,
R3 is hydrogen,
and none of R1, R2 and R3 is bound to the 10-position of the pyrrolo[2,1-a]-isoquinoline ring,
and
R4 is hydrogen, or 1-4C-alkyl,
R41 is hydrogen, or 1-4C-alkyl,
R5 is hydrogen, and
R51 is hydrogen;
or
R1 is 1-4C-alkoxy,
R2 is 1-4C-alkoxy,
R3 is hydrogen,
and none of R1, R2 and R3 is bound to the 10-position of the pyrrolo[2,1-a]-isoquinoline ring,
and
R4 is 1-4C-alkyl,
R41 is hydrogen, or 1-4C-alkyl,
R5 is hydrogen, and
R51 is hydrogen;
R6 is 1-4C-alkyl,
R7 is 4-hydroxy-3,5-dimethylphenyl, 3-dimethylamino-phenyl, or
pyridyl, indolyl, thiophenyl, quinolinyl or naphthyl,
R8 is —C(O)—OR9, in which
R9 is 1-2C-alkyl,
or a salt, stereoisomer, hydrate or hydrate of a salt thereof.

10. A compound of formula I according to claim 1,
in which, in a first embodiment,
either
R1 is bonded in the 8-position of the pyrrolo[2,1-a]-isoquinoline ring, and is chlorine, 2-methoxy-ethoxy or difluoromethoxy, and
R2 is bonded in the 9-position of the pyrrolo[2,1-a]-isoquinoline ring, and is methoxy,
or
R1 is bonded in the 9-position of the pyrrolo[2,1-a]-isoquinoline ring, and is chlorine, fluorine, methyl, nitro, amino or difluoromethoxy, and
R2 is bonded in the 8-position of the pyrrolo[2,1-a]-isoquinoline ring, and is methoxy,
and
R3 is hydrogen,
and
either
R4 is hydrogen,
R41 is hydrogen,
R5 is hydrogen, and
R51 is hydrogen,
or
R4 is methyl,
R41 is hydrogen or methyl,
R5 is hydrogen, and
R51 is hydrogen;
or in which, in a second embodiment,
R1 is bonded in the 8-position of the pyrrolo[2,1-a]-isoquinoline ring, and is methoxy,
R2 is bonded in the 9-position of the pyrrolo[2,1-a]-isoquinoline ring, and is methoxy,
R3 is hydrogen,
and
R4 is methyl,
R41 is hydrogen or methyl,
R5 is hydrogen,
R51 is hydrogen;
R6 is methyl or 2-methoxycarbonylethyl,
R7 is 4-hydroxy-3,5-dimethylphenyl, 3-dimethylamino-phenyl, 3,4,5-trimethoxyphenyl, quinolinyl or naphthyl,
R8 is phenylcarbonyl, —C(O)—N(R82)R83 or —C(O)—OR9, in
which
R82 is hydrogen, methyl, ethyl, iso-propyl, iso-butyl, cyclohexyl, cyclopropyl or phenyl, and
R83 is hydrogen or methyl, or
R82 and R83 together and with inclusion of the nitrogen atom, to which they are bound, form a pyrrolidinyl radical,
R9 is methyl or ethyl,
or a salt, stereoisomer, hydrate or hydrate of a salt thereof.

11. A compound according to claim 1, wherein said compounds have the formula I,
in which
R1 is 1-4C-alkoxy,
R2 is 1-4C-alkoxy,
R3 is hydrogen,
and none of R1 and R2 is bound to the 7- or 10-position of the pyrrolo[2,1-a]-isoquinoline ring,
and in which
R4 is 1-4C-alkyl,
R41 is hydrogen, or 1-4C-alkyl,
R5 is hydrogen,
R51 is hydrogen,
or a salt, stereoisomer, hydrate or hydrate of a salt thereof.

12. A compound according to claim 1, wherein said compounds have the formula I,
in which
either
R1 is halogen, nitro, amino, mono- or di-1-4C-alkylamino, 1-4C-alkyl, 1-4C-alkoxy-2-4C-alkoxy, 3-7C-cycloalkoxy, 3-7C-cycloalkylmethoxy, or completely or predominantly fluorine-substituted 1-4C-alkoxy, and
R2 is 1-4C-alkoxy,
or
R1 is 1-4C-alkoxy, 1-4C-alkoxy-2-4C-alkoxy, 3-7C-cycloalkoxy, 3-7C-cycloalkylmethoxy, or completely or predominantly fluorine-substituted 1-4C-alkoxy, and
R2 is halogen,
and
R3 is hydrogen,
and none of R1 and R2 is bound to the 7- or 10-position of the pyrrolo[2,1-a]-isoquinoline ring;
and in which
either
R4 is hydrogen,
R41 is hydrogen,
R5 is hydrogen, and
R51 is hydrogen,
or
R4 is 1-4C-alkyl,
R41 is hydrogen or 1-4C-alkyl,
R5 is hydrogen, and
R51 is hydrogen;
or a salt, stereoisomer, hydrate or hydrate of a salt thereof.

13. A compound according to claim 1, wherein said compounds have the formula I,
in which
either
R1 is halogen, nitro, amino, mono- or di-1-4C-alkylamino, 1-4C-alkyl, 1-4C-alkoxy, 1-4C-alkoxy-2-4C-alkoxy, 3-7C-cycloalkoxy, 3-7C-cycloalkylmethoxy, or completely or predominantly fluorine-substituted 1-4C-alkoxy, and
R2 is 1-4C-alkoxy,
or
R1 is 1-4C-alkoxy, 1-4C-alkoxy-2-4C-alkoxy, 3-7C-cycloalkoxy, 3-7C-cycloalkylmethoxy, or completely or predominantly fluorine-substituted 1-4C-alkoxy, and
R2 is halogen,
and
R3 is 1-4C-alkoxy, and none of R1, R2 and R3 is bound to the 10-position of the pyrrolo[2,1-a]-isoquinoline ring;
and in which
either
R4 is hydrogen,
R41 is hydrogen,
R5 is hydrogen, and
R51 is hydrogen,
or
R4 is 1-4C-alkyl,
R41 is hydrogen or 1-4C-alkyl,
R5 is hydrogen, and
R51 is hydrogen;
or a salt, stereoisomer, hydrate or hydrate of a salt thereof.

14. A compound according to claim 1, wherein said compounds have the formula I,
in which
R1 is halogen, 1-4C-alkoxy, 1-4C-alkoxy-2-4C-alkoxy, or completely or predominantly fluorine-substituted 1-4C-alkoxy,
R2 is 1-4C-alkoxy,
R3 is 1-4C-alkoxy,
and none of R1, R2 and R3 is bound to the 10-position of the pyrrolo[2,1-a]-isoquinoline ring,
and
R4 is hydrogen, or 1-4C-alkyl,
R41 is hydrogen, or 1-4C-alkyl,
R5 is hydrogen, and
R51 is hydrogen;
or
R1 is halogen, 1-4C-alkoxy-2-4C-alkoxy, or completely or predominantly fluorine-substituted 1-4C-alkoxy,
R2 is 1-4C-alkoxy,
R3 is hydrogen,
and none of R1, R2 and R3 is bound to the 10-position of the pyrrolo[2,1-a]-isoquinoline ring,
and
R4 is hydrogen, or 1-4C-alkyl,
R41 is hydrogen, or 1-4C-alkyl,
R5 is hydrogen, and
R51 is hydrogen;
or
R1 is 1-4C-alkoxy,
R2 is 1-4C-alkoxy,
R3 is hydrogen,
and none of R1, R2 and R3 is bound to the 10-position of the pyrrolo[2,1-a]-isoquinoline ring,
and
R4 is 1-4C-alkyl,
R41 is hydrogen, or 1-4C-alkyl,
R5 is hydrogen, and
R51 is hydrogen;
and
R6 is 1-4C-alkyl;
or a salt, stereoisomer, hydrate or hydrate of a salt thereof.

15. A compound according to claim 1, wherein said compounds have the formula I,
in which
R1 is fluorine, chlorine, 1-2C-alkoxy-2-3C-alkoxy, or completely or predominantly fluorine-substituted 1-2C-alkoxy,
R2 is 1-2C-alkoxy,
R3 is hydrogen,
and none of R1 and R2 is bound to the 7- or 10-position of the pyrrolo[2,1-a]-isoquinoline ring,
and
R4 is hydrogen,
R41 is hydrogen,
R5 is hydrogen, and
R51 is hydrogen;
or
R1 is 1-2C-alkoxy,
R2 is 1-2C-alkoxy,
R3 is hydrogen,
and none of R1 and R2 is bound to the 7- or 10-position of the pyrrolo[2,1-a]-isoquinoline ring,
and
R4 is 1-2C-alkyl,
R41 is hydrogen, or 1-2C-alkyl,
R5 is hydrogen, and
R51 is hydrogen;
or
R1 is fluorine, chlorine, 1-2C-alkoxy-2-3C-alkoxy, or completely or predominantly fluorine-substituted 1-2C-alkoxy,
R2 is 1-2C-alkoxy,
R3 is hydrogen,
and none of R1 and R2 is bound to the 7- or 10-position of the pyrrolo[2,1-a]-isoquinoline ring,
and
R4 is 1-2C-alkyl,
R41 is hydrogen, or 1-2C-alkyl,
R5 is hydrogen, and
R51 is hydrogen;
and
R6 is 1-2C-alkyl;
or a salt, stereoisomer, hydrate or hydrate of a salt thereof.

16. A compound according to claim 1, wherein said compounds have the formula I,
in which
R1 is fluorine, chlorine, 1-2C-alkoxy-2-3C-alkoxy, or completely or predominantly fluorine-substituted 1-2C-alkoxy,
R2 is 1-2C-alkoxy,
R3 is hydrogen,
and none of R1 and R2 is bound to the 7- or 10-position of the pyrrolo[2,1-a]-isoquinoline ring,
and
R4 is 1-2C-alkyl,
R41 is hydrogen, or 1-2C-alkyl,
R5 is hydrogen, and
R51 is hydrogen;
or a salt, stereoisomer, hydrate or hydrate of a salt thereof.

17. A compound according to claim 1, wherein said compounds have the formula I,
in which
R6 is 1-4C-alkyl,
R7 is Het2,4-hydroxy-3,5-dimethylphenyl, 3-dimethylamino-phenyl, or naphthyl, in which
Het2 is a monocyclic or fused bicyclic 5- to 10-membered heteroaryl radical comprising one to three heteroatoms, each of which is selected from the group consisting of nitrogen, oxygen and sulfur,
R8 is —C(O)—OR9, in which
R9 is 1-4C-alkyl,
or a salt, stereoisomer, hydrate or hydrate of a salt thereof.

18. A compound according to claim 1, wherein said compound is selected from the group consisting of:
1. 2-(4-Hydroxy-3,5-dimethyl-phenyl)-8,9-dimethoxy-3,5,6-trimethyl-5,6-dihydro-pyrrolo[2,1-α]isoquinoline-1-carboxylic acid ethyl ester,
2. (6RS)-2-(4-Hydroxy-3,5-dimethyl-phenyl)-8,9-dimethoxy-3,6-dimethyl-5,6-dihydro-pyrrolo[2,1-a]isoquinoline-1-carboxylic acid ethyl ester,
3. (6RS)-8,9-Dimethoxy-3,6-dimethyl-2-(3,4,5-trimethoxy-phenyl)-5,6-dihydro-pyrrolo[2,1-a]isoquinoline-1-carboxylic acid ethyl ester,
4. 9-(1,1-Difluoro-methoxy)-2-(3-dimethylamino-phenyl)-8-methoxy-3-methyl-5,6-dihydro-pyrrolo[2,1-a]-isoquinoline-1-carboxylic acid ethyl ester,
5. 9-(1,1-Difluoro-methoxy)-2-(4-hydroxy-3,5-dimethyl-phenyl)-8-methoxy-3-methyl-5,6-dihydro-pyrrolo[2,1-a]-isoquinoline-1-carboxylic acid ethyl ester,
6. 9-(1,1-Difluoro-methoxy)-8-methoxy-3-methyl-2-(3,4,5-trimethoxy-phenyl)-5,6-dihydro-pyrrolo[2,1-a]-isoquinoline-1-carboxylic acid ethyl ester,
7. 2-(4-Hydroxy-3,5-dimethyl-phenyl)-8,9,10-trimethoxy-3-methyl-5,6-dihydro-pyrrolo[2,1-a]isoquinoline-1-carboxylic acid ethyl ester,
8. 8-(1,1-Difluoro-methoxy)-9-methoxy-3-methyl-2-(3,4,5-trimethoxy-phenyl)-5,6-dihydro-pyrrolo[2,1-a]-isoquinoline-1-carboxylic acid ethyl ester,
9. 8-(1,1-Difluoro-methoxy)-2-(4-hydroxy-3,5-dimethyl-phenyl)-9-methoxy-3-methyl-5,6-dihydro-pyrrolo[2,1-a]-isoquinoline-1-carboxylic acid ethyl ester,
10. 8-(1,1-Difluoro-methoxy)-2-(3-dimethylamino-phenyl)-9-methoxy-3-methyl-5,6-dihydro-pyrrolo[2,1-a]-isoquinoline-1-carboxylic acid ethyl ester,
11. 8,9-(1,1-Difluoro-methylenedioxy)-2-(3-dimethylamino-phenyl)-3-methyl-5,6-dihydro-pyrrolo[2,1-a]-isoquinoline-1-carboxylic acid ethyl ester,
12. 8,9-(1,1-Difluoro-methylenedioxy)-2-(4-hydroxy-3,5-dimethyl-phenyl)-3-methyl-5,6-dihydro-pyrrolo[2,1-a]-isoquinoline-1-carboxylic acid ethyl ester,
13. 8,9-(1,1-Difluoro-methylenedioxy)-3-methyl-2-(3,4,5-trimethoxy-phenyl)-5,6-dihydro-pyrrolo[2,1-a]-isoquinoline-1-carboxylic acid ethyl ester,
14. 9-Chloro-2-(4-hydroxy-3,5-dimethyl-phenyl)-8-methoxy-3-methyl-5,6-dihydro-pyrrolo[2,1-a]isoquinoline-1-carboxylic acid ethyl ester,
15. 9-Chloro-8-methoxy-3-methyl-2-(3,4,5-trimethoxy-phenyl)-5,6-dihydro-pyrrolo[2,1-a]isoquinoline-1-carboxylic acid ethyl ester,
16. 9-Chloro-2-(3-dimethylamino-phenyl)-8-methoxy-3-methyl-5,6-dihydro-pyrrolo[2,1-a]isoquinoline-1-carboxylic acid ethyl ester,
17. 8-Chloro-2-(4-hydroxy-3,5-dimethyl-phenyl)-9-methoxy-3-methyl-5,6-dihydro-pyrrolo[2,1-a]isoquinoline-1-carboxylic acid ethyl ester,
18. 2-(4-Hydroxy-3,5-dimethyl-phenyl)-9-methoxy-8-(2-methoxy-ethoxy)-3-methyl-5,6-dihydro-pyrrolo[2,1-a]isoquinoline-1-carboxylic acid ethyl ester,
19. 9-Methoxy-8-(2-methoxy-ethoxy)-3-methyl-2-(3,4,5-trimethoxy-phenyl)-5,6-dihydro-pyrrolo[2,1-a]isoquinoline-1-carboxylic acid ethyl ester,
20. 9-Methoxy-8-(2-methoxy-ethoxy)-3-methyl-2-naphthalen-1-yl-5,6-dihydro-pyrrolo[2,1-a]isoquinoline-1-carboxylic acid ethyl ester,
21. 9-Fluoro-2-(4-hydroxy-3,5-dimethyl-phenyl)-8-methoxy-3-methyl-5,6-dihydro-pyrrolo[2,1-a]isoquinoline-1-carboxylic acid ethyl ester,
22. 2-(4-Hydroxy-3,5-dimethyl-phenyl)-8-methoxy-3-methyl-9-nitro-5,6-dihydro-pyrrolo[2,1-a]isoquinoline-1-carboxylic acid ethyl ester,
23. 2-(4-Hydroxy-3,5-dimethyl-phenyl)-8-methoxy-3,9-dimethyl-5,6-dihydro-pyrrolo[2,1-a]isoquinoline-1-carboxylic acid ethyl ester,
24. 8,9-Dimethoxy-3-(2-methoxycarbonyl-ethyl)-6,6-dimethyl-2-quinolin-4-yl-5,6-dihydro-pyrrolo[2,1-a]isoquinoline-1-carboxylic acid ethyl ester,
25. 9-Amino-2-(4-hydroxy-3,5-dimethyl-phenyl)-8-methoxy-3-methyl-5,6-dihydro-pyrrolo[2,1-a]isoquinoline-1-carboxylic acid ethyl ester,
26. 1-[2-(4-Hydroxy-3,5-dimethyl-phenyl)-8,9-dimethoxy-3-methyl-5,6-dihydro-pyrrolo[2,1-a]isoquinolin-1-yl]-1-phenyl-methanone,
27. 4-(8,9-Dimethoxy-3-methyl-1-phenyl-5,6-dihydro-pyrrolo[2,1-a]isoquinolin-2-yl)-2,6-dimethyl-phenol,
28. 2-(4-Hydroxy-3,5-dimethyl-phenyl)-8,9-dimethoxy-3-methyl-5,6-dihydro-pyrrolo[2,1-a]isoquinoline-1-carboxylic acid cyclohexyl amide,
29. 1-[2-(4-Hydroxy-3,5-dimethyl-phenyl)-8,9-dimethoxy-3-methyl-5,6-dihydro-pyrrolo[2,1-a]isoquinolin-1-yl]-1-pyrrollidin-1-yl-methanone,
30. 2-(4-Hydroxy-3,5-dimethyl-phenyl)-8,9-dimethoxy-3-methyl-5,6-dihydro-pyrrolo[2,1-a]isoquinoline-1-carboxylic acid isopropylamide,
31. 2-(4-Hydroxy-3,5-dimethyl-phenyl)-8,9-dimethoxy-3-methyl-5,6-dihydro-pyrrolo[2,1-a]isoquinoline-1-carboxylic acid dimethylamide,
32. 2-(4-Hydroxy-3,5-dimethyl-phenyl)-8,9-dimethoxy-3-methyl-5,6-dihydro-pyrrolo[2,1-a]isoquinoline-1-carboxylic acid methylamide,
33. 2-(4-Hydroxy-3,5-dimethyl-phenyl)-8,9-dimethoxy-3-methyl-5,6-dihydro-pyrrolo[2,1-a]isoquinoline-1-carboxylic acid amide,
34. 2-(4-Hydroxy-3,5-dimethyl-phenyl)-8,9-dimethoxy-3-methyl-5,6-dihydro-pyrrolo[2,1-a]isoquinoline-1-carboxylic acid phenylamide,
35. 2-(4-Hydroxy-3,5-dimethyl-phenyl)-8,9-dimethoxy-3-methyl-5,6-dihydro-pyrrolo[2,1-a]isoquinoline-1-carboxylic acid ethylamide,
36. 2-(4-Hydroxy-3,5-dimethyl-phenyl)-8,9-dimethoxy-3-methyl-5,6-dihydro-pyrrolo[2,1-a]isoquinoline-1-carboxylic acid sec-butylamide, and
37. 2-(4-Hydroxy-3,5-dimethyl-phenyl)-8,9-dimethoxy-3-methyl-5,6-dihydro-pyrrolo[2,1-a]isoquinoline-1-carboxylic acid cyclopropylamide;
and the salts, stereoisomers, hydrates, and hydrates of the salts thereof.

19-20. (canceled)

21. A pharmaceutical composition comprising one or more compounds according to claim 1, or a pharmaceutically acceptable salt, stereoisomer, hydrate or hydrate of a salt thereof, together with a customary pharmaceutical excipient and/or vehicle.

22. A method for treating hyperproliferative diseases of benign or malignant behaviour and/or disorders responsive to the induction of apoptosis, in a patient comprising administering to said patient a therapeutically effective amount of a compound according to claim 1, or a pharmaceutically acceptable salt, stereoisomer, hydrate or hydrate of a salt thereof.

23. The method according to claim 22, wherein said hyperproliferative disease of benign or malignant behavior and/or disorder responsive to the induction of apoptosis is cancer.
